(19) United States
(12) Patent Application Publication
(10) Pub. No.: US 2026/0139009 A1
(43) Pub. Date: May 21, 2026

(54) PREFUSION-STABILIZED CMV GB PROTEINS

(71) Applicant: BOARD OF REGENTS, THE UNIVERSITY OF TEXAS SYSTEM, Austin, TX (US)

(72) Inventors: Jason MCLELLAN, Austin, TX (US); Madeline SPONHOLTZ, Austin, TX (US); Patrick BYRNE, Austin, TX (US); Ching-Lin HSIEH, Austin, TX (US); Ling ZHOU, Austin, TX (US); Ryan MCCOOL, Austin, TX (US); Nicole JOHNSON, Austin, TX (US); Jory GOLDSMITH, Austin, TX (US); Ajit RAMAMOHAN, Austin, TX (US)

(73) Assignee: BOARD OF REGENTS, THE UNIVERSITY OF TEXAS SYSTEM, Austin, TX (US)

(21) Appl. No.: 19/107,808

(22) PCT Filed: Sep. 1, 2023
(86) PCT No.: PCT/US2023/073369

§ 371 Date: Feb. 28, 2025

Related U.S. Application Data
(60) Provisional application No. 63/374,482, filed on Sep. 2, 2022.

(51) Int. Cl. C07K 14/005; A61K 39/00

(52) U.S. Cl. CPC C07K 14/005; A61K 39/00; C07K 2319/03; C12N 2710/16122

(57) ABSTRACT

Provided herein are engineered hCMV gB polypeptides. In some aspects, the engineered gB polypeptides exhibit enhanced conformational stability and/or antigenicity. Methods are also provided for use of the engineered gB polypeptides as diagnostics, in screening platforms, and/or in vaccine compositions.

### REFERENCE TO RELATED APPLICATIONS

The present application claims the priority benefit of U.S. provisional application No. 63/374,482, filed Sep. 2, 2022, the entire contents of which are incorporated herein by reference.

### REFERENCE TO A SEQUENCE LISTING

This application contains a Sequence Listing XML, which has been submitted electronically and is hereby incorporated by reference in its entirety. Said Sequence Listing XML, created on Sep. 1, 2023, is named UTFBP1312WO_ST26.xml and is 147,066 bytes in size.

### BACKGROUND

### 1. Field

The present disclosure relates generally to the fields of medicine, virology, and immunology. More particularly, it concerns engineered human cytomegalovirus (CMV) gB polypeptides and uses thereof.

### 2. Description of Related Art

Human cytomegalovirus (CMV) is a double stranded DNA virus of the (3-herpesvirus family. CMV is the leading cause of congenital and neonatal hearing loss resulting from vertical virus transmission following infection or reactivation of latent virus in pregnant women. In addition, CMV is a common opportunistic pathogen affecting immunosuppressed patients, such as solid organ and stem cell transplant patients, AIDS patients, etc. Though development of a vaccine against CMV has been listed as a top priority by the Institute of Medicine, none has been licensed to date.

The CMV genome encodes several envelope glycoproteins, one of which is glycoprotein B (gB). Glycoprotein B is a fusogen that is required for virus entry into cells and an important target for neutralizing antibody (nAb) responses to infection. CMV vaccines that incorporate gB subunit antigens have been under development. Clinical studies have shown that some gB subunit-based vaccine candidates are safe and immunogenic, though improvements in protective efficacy and durability of protection are desirable.

Accordingly, safe and effective immunogenic compositions to protect against CMV infection are needed. Diagnostic reagents to detect immune responses to CMV, to guide the design of gB-based CMV vaccines, and to support the development of therapeutic or prophylactic antibodies against CMV are also needed.

### SUMMARY

As such, provided herein are engineered proteins having at least one amino acid mutation relative to the amino acid sequence of a wild-type HCMV gB protein, wherein the engineered proteins are stabilized in the prefusion conformation of HCMV gB. The engineered proteins may specifically bind to a CMV gB prefusion-specific antibody.

In one embodiment, provided herein are engineered human cytomegalovirus (HCMV) gB protein ectodomains comprising a sequence having at least 90% identity to amino acids 23-704 of SEQ ID NO: 1, said engineered protein comprising at least one substitution or set of substitutions selected from the group consisting of: Y89C/N599C; Y89C/G622C; V93C/V552C; V93C/L603C; A97C/R540C; A97C/A549C; G99C/L616C; R104C/I642C; R104C/T644C; E106C/T644C; L121C/N496C; M126C/W431C; V128C/V429C; V134C/I653C; A135C/I653C; Y153C/Y160C; Y153C/Y242C; Y160C/Y708C; N220C/E657C; T221C/T553C; H222C/E657C; K260C/S641C; V273C/L616C; T347C/A650C; I356C/A500C; M371C/A505C; Y415C/G433C; Y415C/K435C; F421C/V429C; G543C/K617C; G543C/F619C; D544C/Q597C; D544C/I620C; V545C/I618C; G604C/Y667C; Q612C/R662C; T644C/S647C; F661C/Y218C; S674C/E698C; D679C/R685C; D679C/E686C; E698C/R673C; N750C/G753C; L102W; I103Y; T112L; T112Y; V127F; K130W; K130Y; K130L; T159W; L162W; S223V/T253V/T270M/T659V; T253L; K260W; A267M; T270W; V273F; R354W; I356F; S367M; A396V; G433H; A500M; A500L/A503S; F541W; N556Y; E597F; E597Y; N599Y; R607Y; S641F; V645L; L616W; I620F; E627F; S643W; D646W; S647L; R662W; L664F; D679V/E682N; R685L/R607M; E686M; E686L/K691M; S689Y; G711L; L715F; L715F/V741Y/N750W; G731M; A738M; V741Y; I103R; V110E; D120R; Y153R/Y712E; Y155E; Y155E/G735K; Y155E/L709K; Y168E; Y218D/F584K; W240R; W240R/A732E; L241R/V728E; Y242D/L715K; V273K; I305E; R354E; E359R; R497E; Q501E; S526K; Y592E; N605E; H606E; L613D; P614R; D630R; M648E; A650D; I653D; D654E; L656D; V702E; K710E; V733D/F752K; K749E; N750K; E119P; L121P; D122P; Y129P; A373P; A257P; K340P; N341P; A351P; G426P; L427P; H477P; V645P; A650P; L651P; E671P; L672P; 1683P; M684P; E686P; K691P; G720P/A721P/A722P; A725P/V726P/A727P; Q98L/N658I; T100L/A267I; R104I; Y153F; L161E; L161R; L161W; E1671; F198W; T245E; T253L/T270L; D272N; A373F; D509L; D509R; R511Q; R512Q; R512W; S641E; T659V/N220F; E686N; R693L; R693L/K700V; and K700V, wherein the position are relative to SEQ ID NO: 1.

In some aspects, the engineered HCMV gB protein ectodomains comprise or further comprises at least one set of paired cysteine substitutions selected from the group consisting of: Y89C/N599C; Y89C/G622C; V93C/V552C; V93C/L603C; A97C/R540C; A97C/A549C; G99C/L616C; R104C/I642C; R104C/T644C; E106C/T644C; L121C/N496C; M126C/W431C; V128C/V429C; V134C/I653C; A135C/I653C; Y153C/Y160C; Y153C/Y242C; Y160C/Y708C; N220C/E657C; T221C/T553C; H222C/E657C; K260C/S641C; V273C/L616C; T347C/A650C; I356C/A500C; M371C/A505C; Y415C/G433C; Y415C/K435C; F421C/V429C; G543C/K617C; G543C/F619C; D544C/Q597C; D544C/1620C; V545C/I618C; G604C/Y667C; Q612C/R662C; T644C/S647C; F661C/Y218C; S674C/E698C; D679C/R685C; D679C/E686C; E698C/R673C; E698C/S674C; and N750C/G753C. In some aspects, the engineered proteins further comprise at least one set of paired cysteine substitutions selected from the group consisting of: Q98C/N658C; L162C/M716C; S219C/N586C; S219C/A585C; W240C/G735C; Y242C/G731C G271C/P614C; W349C/A650C; S367C/A503C; L548C/P655C; A549C/N658C; S550C/P655C; S550C/E657C; and G604C/L672C. In some aspects, the paired cysteine substitutions form a disulfide bond.

In some aspects, the engineered HCMV gB protein ectodomains comprise or further comprise at least one cavity filling substitution or set of cavity filling substitutions selected from the group consisting of: L102W; I103Y; T112L; T112Y; K130W; L162W; V127F; K130Y; K130L; T159W; S223V/T253V/T270M/T659V; T253L; K260W; A267M; T270W; V273F; R354W; 1356F; S367M; A396V; G433H; A500M; A500L/A503S; F541W; N556Y; E597F; E597Y; N599Y; R607Y; S641F; V645L; L616W; I620F; E627F; S643W; D646W; S647L; R662W; L664F; D679V/E682N; R685L/R607M; E686M; E686L/K691M; S689Y; G711L; L715F; L715F/V741Y/N750W; G731M; A738M; and V741Y.

In some aspects, the engineered HCMV gB protein ectodomains comprise or further comprise at least one substitution or set of substitutions selected from the group consisting of: I103R; V110E; D120R; Y153R/Y712E; Y155E; Y155E/G735K; Y155E/L709K; Y168E; Y218D/F584K; W240R; W240R/A732E; L241R/V728E; Y242D/L715K; V273K; 1305E; R354E; E359R; R497E; Q501E; S526K; Y592E; N605E; H606E; L613D; P614R; D630R; M648E; A650D; I653D; D654E; L656D; V702E; K710E; V733D/F752K; K749E; and N750K. In some aspects, the substitution forms a salt bridge within the pair of substitutions or between the single substitution and a native amino acid in the protein.

In some aspects, the engineered HCMV gB protein ectodomains comprise or further comprise at least one substitution or set of substitutions selected from the group consisting of: E119P; L121P; D122P; Y129P; A373P; A257P; K340P; N341P; A351P; G426P; L427P; H477P; V645P; A650P; L651P; E671P; L672P; I683P; M684P; E686P; K691P; G720P/A721P/A722P; and A725P/V726P/A727P.

In some aspects, the engineered HCMV gB protein ectodomains further comprise at least one substitution selected from the group consisting of: N478P; L479P; V480P; Y481P; A482P; Q483P; L484P; and D646P.

In some aspects, the engineered HCMV gB protein ectodomains comprise or further comprise at least one substitution or set of substitutions selected from the group consisting of: Q98L/N658I; T100L/A267I; R104I; Y153F; L161E; L161R; L161W; E167I; F198W; T245E; T253L/T270L; D272N; A373F; D509L; D509R; R511Q; R512Q; R512W; S641E; T659V/N220F; E686N; R693L; R693L/K700V; and K700V.

In some aspects, the engineered HCMV gB protein ectodomains comprise or further comprise at least one substitution selected from the group consisting of: Y155G; I156H; Y157R; W240A; L241F; L241T; Y242H; C246S; R457S; R460S; D509A; and E686Q. In some aspects, the engineered proteins comprise C246S; R457S; and R460S substitutions.

In some aspects, positions 436-489 relative to SEQ ID NO: 1 have been replaced with GGP, GPP, PGP, or GPG. In some aspects, positions 437-486 relative to SEQ ID NO: 1 have been replaced with CGADPQLC (SEQ ID NO: 76) or SGCDPQLC (SEQ ID NO: 77). In some aspects, positions 189-201 relative to SEQ ID NO: 1, positions 280-294 relative to SEQ ID NO: 1, or positions 265-284 relative to SEQ ID NO: 1 are deleted.

In some aspects, positions 439-483 relative to SEQ ID NO: 1 have been replaced with RLNPP (SEQ ID NO: 78) or GADPQ (SEQ ID NO: 79); positions 439-484 relative to SEQ ID NO: 1 have been replaced with GMTPEQ (SEQ ID NO: 80); positions 437-486 relative to SEQ ID NO: 1 have been replaced with YDVCDPQLCY (SEQ ID NO: 81); or positions 439-487 relative to SEQ ID NO: 1 have been replaced with VGPPPTHKI (SEQ ID NO: 82).

In some aspects, the engineered HCMV gB protein ectodomains comprise a combination of at least one engineered disulfide bond and at least one cavity filling substitution. In some aspects, the engineered proteins comprise a combination of at least one engineered disulfide bond and at least one proline substitution.

In some aspects, the engineered HCMV gB protein ectodomains comprise a set of substitutions selected from the group consisting of: V645/L102W/R693L/K700V; V645P/E686L/H222C/E657C; E106C/T644C/R1041/A267I/T100L/R693L/K700V; E106C/T644C/R104I/L102W/H222C/E657C; K130Y/A267I/T100L/R693L/K700V; K130Y/E686L/H222C/E657C; A503C/S367C/L102W/R693L/K700V; A503C/S367C/V273F/H222C/E657C; 1356C/A500C/A267I/T100L/R693L/K700V; H222C/E657C/S674C/E698C; N220C/E657C/S674C/E698C; H222C/E657C/T100L/A267I; N220C/E657C/T100L/A267I; H222C/E657C/K260W; N220C/E657C/K260W; H222C/E657C/V645P; N220C/E657C/V645P; H222C/E657C/E1671; N220C/E657C/E1671; H222C/E657C/V273F; N220C/E657C/V273F; H222C/E657C/L102W; N220C/E657C/L102W; H222C/E657C/Q98L/N658I; N220C/E657C/Q98L/N658I; H222C/E657C/K130Y; N220C/E657C/K130Y; H222C/E657C/N341P; N220C/E657C/N341P; H222C/E657C/R693L/700V; N220C/E657C/R693L/K700V; H222C/E657C/L162W; N220C/E657C/L162W; H222C/E657C/V480P; N220C/E657C/V480P; H222C/E657C/L484P; N220C/E657C/L484P; H222C/E657C/L479P; N220C/E657C/L479P; H222C/E657C/Y481P; N220C/E657C/Y481P; H222C/E657C/Q612C/R662C; N220C/E657C/Q612C/R662C; H222C/E657C/M371C/A505C; N220C/E657C/M371C/A505C; H222C/E657C/K260C/S641C; N220C/E657C/K260C/S641C; H222C/E657C/1356C/A500C; N220C/E657C/I356C/A500C; H222C/E657C/Y160C/Y708C; N220C/E657C/Y160C/Y708C; H222C/E657C/V134C/I653C; N220C/E657C/V134C/I653C; V134C/I653C/K260C/S641C; V134C/I653C/Q612C/R662C; V134C/I653C/S674C/E698C; V134C/I653C/M371C/A505C; V134C/I653C/I356C/A500C; V134C/I653C/Y160C/Y708C; N220C/E657CN341P/L484P/V645P; N220C/E657C/T100L/A267I/Q98L/N658I; N220C/E657C/R693L/K700V/E686L/K691M; N220C/E657C/L102W/K130Y/L162W/K260W/V273F; V134C/I653C/N341P/L484P/V645P; V134C/I653C/T100L/A267I/Q98L/N658I; VI34C/I653C/R693L/K700V/E686L/K691M; V134C/I653C/L102W/K130Y/L162W/K260W/V273F; V134C/I653C/S643W; V134C/I653C/S647L; V134C/I653C/N599Y; V134C/I653C/N566Y; V134C/I653C/E597F; V134C/I653C/E597Y; V134C/I653C/E627F; V134C/I653C/H606E; T100L/A267I/V134C/I653C/H222C/E657C; and V134C/I653C/H222C/E657C.

In some aspects, the engineered HCMV gB protein ectodomains comprise a substitution or set of substitutions selected from any one of the substitutions and sets of substitutions of Tables 1 and 2.

In some aspects, the engineered HCMV gB protein ectodomains comprise an amino acid sequence having at least 91%, at least 92%, at least 93%, at least 94%, at least 95%, at least 96%, at least 97%, at least 98%, or at least 99% identity to amino acids 23-704 of SEQ ID NO: 1. In some aspects, the engineered HCMV gB protein ectodomains comprise at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, or 68 amino acid substitutions relative to the sequence of amino acids 23-704 of SEQ ID NO: 1.

In some aspects, the engineered HCMV gB protein ectodomains comprise an amino acid sequence having at least 91%, at least 92%, at least 93%, at least 94%, at least 95%, at least 96%, at least 97%, at least 98%, or at least 99% identity to amino acids 23-704 of SEQ ID NO: 4. In some aspects, the engineered HCMV gB protein ectodomains comprise at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, or 68 amino acid substitutions relative to the sequence of amino acids 23-704 of SEQ ID NO: 4.

In some aspects, the engineered HCMV gB protein ectodomains comprise an amino acid sequence having at least 91%, at least 92%, at least 93%, at least 94%, at least 95%, at least 96%, at least 97%, at least 98%, or at least 99% identity to the amino acid sequence of SEQ ID NO: 5. In some aspects, the engineered HCMV gB protein ectodomains comprise at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, or 68 amino acid substitutions relative to the amino acid sequence of SEQ ID NO: 5.

In one embodiment, provided herein are engineered proteins comprising an engineered human cytomegalovirus (HCMV) gB protein ectodomain described herein.

In some aspects, the engineered proteins do not comprise the cytoplasmic tail of HCMV gB. In some aspects, the engineered HCMV gB protein ectodomains are fused or conjugated to a trimerization domain. In some aspects, the engineered HCMV gB protein ectodomains are fused to a trimerization domain. In some aspects, the trimerization domain comprises a T4 fibritin trimerization domain (Fd), a GCN4 domain, a 4J4A domain, or a combination thereof. In some aspects, the trimerization domain comprises a sequence selected from the group consisting of: DTYLSAIEDKIEEILSKIYHIENEIARI (SEQ ID NO: 83), LKQIVLRIMEIEARIAKIE (SEQ ID NO: 86), LKQIVLRIMEIEARIAKIEGSGYIPEAPRDGQAYVRKDGEWVLLSTFLG (SEQ ID NO: 87), LKQIVLRIMEIEARIAKIEGSEFNSLKQIVLRIMEIEARIAKIE (SEQ ID NO: 88), LKQIVLRIMEIEARIAKIEGSLKQIVLRIMEIEARIAKIE (SEQ ID NO: 89), LKQIVLRIMEIEARIAKIEGSLELIKLRIMEIEARIAKIEKDRAIL (SEQ ID NO: 90).

In some aspects, the trimerization domain comprises a T4 fibritin trimerization domain (Fd). In some aspects, the trimerization domain comprises a sequence at least 95%, 96%, at least 97%, at least 98%, or at least 99% identical to the amino acid sequence of SEQ ID NO: 84. In some aspects, the trimerization domain comprises the amino acid sequence of SEQ ID NO: 84. In some aspects, the T4 fibritin trimerization domain (Fd) is fused to the C-terminus of the ectodomain via a Gly-Ser linker.

In some aspects, the engineered HCMV gB protein ectodomains are fused or conjugated to a transmembrane domain. In some aspects, the HCMV gB protein ectodomains is fused to a transmembrane domain. In some aspects, the transmembrane domain comprises a HCMV gB protein transmembrane domain. In some aspects, the transmembrane domain does not comprise a HCMV gB protein transmembrane domain.

In some aspects, the engineered proteins comprise an N-terminal signal sequence. In some aspects, the engineered proteins exhibit improved solubility or stability, as compared to a native gB in a postfusion conformation. In some aspects, the engineered proteins are immunogenic.

In some aspects, the engineered proteins comprise an amino acid sequence having at least 91%, at least 92%, at least 93%, at least 94%, at least 95%, at least 96%, at least 97%, at least 98%, or at least 99% identity to amino acids 23-704 of SEQ ID NO: 4. In some aspects, the engineered HCMV gB protein ectodomains comprise at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, or 71 amino acid substitutions relative to the sequence of amino acids 23-734 of SEQ ID NO: 4.

In some aspects, the engineered proteins comprise an amino acid sequence having at least 91%, at least 92%, at least 93%, at least 94%, at least 95%, at least 96%, at least 97%, at least 98%, or at least 99% identity to the amino acid sequence of SEQ ID NO: 6. In some aspects, the engineered HCMV gB protein ectodomains comprise at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, or 71 amino acid substitutions relative to the amino acid sequence of SEQ ID NO: 6.

In one embodiment, provided herein are engineered human cytomegalovirus (HCMV) gB protein trimers comprising three engineered ectodomains or engineered proteins as provided herein. In some aspects, the trimers are stabilized in a prefusion conformation relative to a trimer of wild-type HCMV gB protein subunits. In some aspects, the trimers comprise at least one engineered disulfide bond between subunits. In some aspects, the trimer comprises at least one engineered disulfide bond between subunits selected from the group consisting of: Y89C/G622C; Q98C/N658C; G99C/L616C; R104C/I642C; E106C/T644C; V134C/I653C; A135C/I653C; Y160C/Y708C; S219C/A585C; N220C/E657C; H222C/E657C; K260C/S641C; G271C/P614C; V273C/L616C; T347C/A650C; W349C/A650C; M371C/A505C; G543C/K617C; G543C/F619C; G543C/K617C; D544C/Q597C; D544C/1620C; V545C/1618C; L548C/P655C; A549C/N658C; S550C/P655C; S550C/E657C; G604C/Y667C; G604C/L672C; F661C/Y218C; S674C/E698C; E698C/R673C; and E698C/S674C.

In one embodiment, provided herein are nucleic acid molecules comprising a nucleotide sequence that encodes an amino acid sequence of any engineered ectodomain or engineered protein provided herein. In some aspects, the nucleic acid molecule further comprises a DNA expression vector. In some aspects, the nucleic acid molecule is an mRNA. In some aspects, the nucleic acid molecule is a self-replicated RNA molecule. In some aspects, the nucleic acid further comprises at least one chemical modification. In some aspects, the at least one chemical modification is selected from the group consisting of pseudouridine, N1-methylpseudouridine, N1-ethylpseudouridine, N1-ethylpseudouridine, 2-thiouridine, 4′-thiouridine, 5-methyleytosine, 2-thio-1-methyl-1-deaza-pseudouri dine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methoxyuridine and 2′-0-methyl uridine.

In one embodiment, provided herein are pharmaceutical compositions comprising (i) an engineered ectodomain or engineered protein provided herein, (ii) an engineered trimer provided herein, or (iii) a nucleic acid molecule provided herein; and a pharmaceutically acceptable carrier. In some aspects, the composition further comprises an adjuvant. In some aspects, the composition further comprises an HCMV antigen. In some aspects, the composition further comprises any one of the following polypeptides: gO, gH, gL, pUL128, pUL130, pUL131, and any combination thereof. In some aspects, the composition is formulated within a cationic lipid nanoparticle.

In one embodiment, provided herein are methods of preventing human cytomegalovirus (HCMV) infection or a disease associated with HCMV infection in a subject, comprising administering to the subject an effective amount of a pharmaceutical composition provided herein.

In one embodiment, provided herein are methods of eliciting an immune response in a subject, the method comprising administering to the subject an effective amount of a pharmaceutical composition provided herein.

In one embodiment, provided herein are methods for reducing cytomegalovirus viral shedding in a subject, the method comprising administering to the subject an effective amount of a pharmaceutical composition provided herein.

In one embodiment, a pharmaceutical composition provided herein is for use in the treatment or prevention of a human cytomegalovirus (HCMV) infection or a disease associated with HCMV infection in a subject.

In aspects of various embodiment, the subject is a mammal.

In one embodiment, a pharmaceutical composition provided herein is for use in eliciting an immune response against cytomegalovirus.

In one embodiment, a pharmaceutical composition provided herein is to be used in the manufacture of a medicament for the treatment or prevention of a human cytomegalovirus (HCMV) infection or a disease associated with HCMV infection.

In one embodiment, provided herein are compositions comprising (i) an engineered ectodomain or engineered protein provided herein or (ii) an engineered trimer provided herein bound to an antibody. In some aspects, the antibody specifically binds to an HCMV gB in the prefusion conformation.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-1G. Characterization of HCMV gB variants. (FIG. 2A) SDS-PAGE and size-exclusion chromatography traces of purified HCMV gB variants, grouped by type (FIG. 1A-1C, single variant designs; FIG. 1D, disulfides; FIG. 1E, salt bridge and charge reduction; FIG. 1F, Apex designs; FIG. 1G, Trimerization motifs). In FIG. 1C, the lines represent, from top to bottom at 11 mL, ECD Base, JSM-10, JSM-9, JG-1, and DW-1. In FIG. 1D, the lines represent, from top to bottom at 12 mL, ECD Base, MS-68, Pf-44, MS-71, MS-29, CL-33, and MS-7. In FIG. 1G, the lines represent, from top to bottom at 14 mL, 4J4A, Foldon, and GCN4. Molecular weight standards for SDS-PAGE are indicated at the left in kDa.

FIGS. 2A-2C. Cryo-electron microscopy (cryo-EM) images and 2D class averages of HCMV gB particles. (FIG. 2A) Images of MS-29 bound to 7H3 Fab. (FIG. 2B) Images of PB-21 bound to 7H3 Fab and 1G2 Fab. (FIG. 2C) Images of PB-22 bound to 1G2 Fab.

FIGS. 3A-3B. Structural analysis of MS-29. (FIG. 3A) Cryo-EM map with a model of HCMV gB ectodomain in a prefusion conformation (PDB accession code 7KDP) fit into the map. (FIG. 3B) Binding interface between E657C of protomer 1 and H222C of protomer 2 modeled as spheres.

FIGS. 4A-4C. Structural analysis of PB-21. (FIG. 4A) Negative stain map of PB-21 bound to 1G2 Fab. (FIG. 4B) Cryo EM structure of PB-21 bound to 7H3 Fab and 1G2 Fab. (FIG. 4C) Binding interface between I653C of protomer 1 and V134C of protomer 2 modeled as spheres.

FIGS. 5A-5B. Structural analysis of PB-22. (FIG. 5A) Negative stain map of PB-22 bound to 1G2 Fab with and without C3 symmetry imposed during 3D reconstruction. (FIG. 5B) Cryo-EM structure of PB-22 bound to 1G2 Fab; binding interface between E657C of protomer 1 and N220C of protomer 2 modeled as spheres.

FIGS. 6A-6C. (FIG. 6A) Primary sequence diagram of wildtype (WT) HCMV gB and the ectodomain (ECD) base construct. Native disulfide bonds are indicated by brackets and N-linked glycosylation sites are shown as branched lines. The N-terminal signal sequence (SS) is shown as a white box. The native furin cleavage site is shown in black. Thick black lines denote the ECD base boundaries, extending from the first residue of the signal sequence to residue 704 of wildtype HCMV gB and followed by the C-terminal Foldon (Fd) trimerization motif and affinity tags, the location of which is indicated below the wildtype primary sequence. In the ECD base construct, serine is substituted at residues 246, 457 and 460 to remove an unpaired, non-conserved cysteine and the furin cleavage site. (FIG. 6B) Exemplary substitutions for HCMV gB stabilization. Shown are examples for four types of designs (proline, salt bridge, disulfide, cavity filling) modeled as spheres on trimeric HCMV gB in a prefusion conformation (PDB accession code 7KDP). One protomer is shown as a ribbon diagram, the second is shown as a cartoon tube trace of the α-carbon backbone, and the third is shown as a transparent surface. The approximate location of the viral membrane is shown as two dashed horizontal lines. Insets highlight the positions of exemlary substitutions, with side chains shown as sticks, disulfide bonds shown as light dashed lines, and hydrogen bonds shown as black dashed lines. (FIG. 6C) Relative expression levels of individual variants in HEK293F medium, calculated by quantitative biolayer interferometry. Variants are grouped by design type. The lower horizontal dotted line indicates the calculated expression of the ECD base construct, and the upper dotted line indicates a 3-fold increase relative to the base construct. Error bars show the standard error of the mean for three independent biological replicates.

FIGS. 7A-7C. (FIG. 7A) SDS-PAGE of single disulfide variants. The upper and lower gels show HCMV gB protein purified by strep-tag affinity chromatography and analyzed by reducing and non-reducing SDS-PAGE, respectively [Coomassie stain]. The individual designs are indicated at the top of each lane. Molecular weight standards are included at the left of each gel. The black, gray, and white arrows to the right of the non-reducing gel indicate the positions of high-, medium- and low-molecular weight species, respectively. (FIG. 7B) Size exclusion chromatography of HCMV gB individual disulfide designs. Superose 6 increase 10/300 size exclusion elution profiles for a set of individual design variants, with the absorbance units at 280 nanometers [mAU] plotted as a function of elution volume in milliliters [mL]. All variants and the ECD base construct were included in the same experiment, and each variant is plotted alongside the same base construct control. The approximate locations of the void volume, peak 1, and peak 2 are indicated with black arrows. (FIG. 7C) Differential scanning fluorimetry (DSF) analysis of HCMV gB individual disulfide design variant thermostability. The first derivative of the fluorescence with respect to temperature (dF/dT) is plotted as a function of temperature. All variants and the ECD base construct were included in the same experiment, and each variant is plotted alongside the same base construct control.

FIGS. 8A-8D. (FIG. 8A) Relative expression of purified combination designs, calculated by biolayer interferometry. The lower horizontal dotted line indicates the calculated expression level of the ECD base construct, and the upper dotted line indicates a 5-fold increase relative to the base construct. Error bars show the standard error of the mean for three independent biological replicates. (FIG. 8B) SDS-PAGE of combinatorial variants. The upper and lower gels show HCMV gB protein purified by strep-tag affinity chromatography and analyzed by reducing and non-reducing SDS-PAGE, respectively [Coomassie stain]. Combinatorial variant IDs are indicated above each lane. Molecular weight standards are included at the left of each gel. The black, gray, and white arrows to the right of the non-reducing gel indicate the positions of high-, medium- and low-molecular weight species, respectively. (FIG. 8C) Negative stain electron microscopy 2D class averages of HCMV gB single design variants and combinatorial design variants purified from FreeStyle 293 cells. Upper left: gB ECD Base bound to 1G2 Fab and 7H3 Fab. Lower left: PB-21 bound to 1G2 Fab and 7H3 Fab. Upper middle: PB-22 bound to 1 G2 Fab. Lower middle: MS-29 bound to 1G2 Fab. Upper right: gB-107 bound to 1G2 Fab and 7H3 Fab. Lower right: gB-100 bound to 1G2 Fab and 7H3 Fab. (FIG. 8D) DSF analysis of combination design variants thermostability. The first derivative of the fluorescence with respect to temperature (dF/dT) is plotted as a function of temperature. All variants and the base construct were included in the same experiment, and each variant is plotted alongside the same ECD base construct control.

FIGS. 9A-9C. Cryo-EM structure of gB-107 bound to 1G2 and 7H3 Fabs. (FIG. 9A) Side and top views of the Coulomb potential map of combinatorial HCMV gB variant gB-107 complexed with 1G2 and 7H3 Fabs shown above the gB-107 model. One protomer of the model is shown as a ribbon diagram, the second is shown as a cartoon tube trace of the α-carbon backbone, and the third is shown as a transparent surface. The inset shows a zoomed view of the substitutions that comprise design gB-107 with side chains shown as sticks. (FIG. 9B) Side view of HCMV gB-107 bound to 1G2 and 7H3 Fabs shown as a ribbon diagram above a zoomed view of the binding interface between 7H3 and gB-107. 7H3 HC is dark gray, 7H3 LC is white, and gB-107 is light gray. In the zoomed interface, key residues are shown as sticks and hydrogen bonds are shown as black dashed lines. (FIG. 9C) The structure of gB-107 (light gray) is superimposed with the previously published structure of prefusion HCMV gB (dark gray, PDB accession code 7KDP). Side and top views are shown for the superimposition of prefusion HCMV gB both as a trimer on the left and as a single protomer on the right. Shifts in domain arrangement are highlighted with arrows.

### DETAILED DESCRIPTION

Provided herein are engineered HCMV gB fusion proteins that have one or more amino acid substitutions that stabilize the human CMV gB fusion protein in the prefusion conformation. Prefusion CMV gB can be used as a vaccine antigen or reagent to detect and/or isolate antibodies in sera. The prefusion HCMV gB proteins described herein, and the nucleic acids that encode the proteins, may be used, for example, as potential immunogens in an immunogenic composition or a vaccine against HCMV, in a method of inducing an immune response in a subject, and as diagnostic tools, among other uses.

### I. NATIVE HCMV GB

Native HCMV gB is synthesized as a 906 or 907 amino acid polypeptide (depending upon the strain of CMV) that undergoes extensive posttranslational modification, including glycosylation at N- and O-linked sites and cleavage by ubiquitous cellular endoproteases into amino- and carboxy-terminal fragments. The N- and C-terminal fragments of gB, gp116 and gp55, respectively, are covalently connected by disulfide bonds, and the mature, glycosylated gB assumes a trimeric configuration. The gB polypeptide contains a large ectodomain (which is cleaved into gp116 and the ectodomain of gp55), a transmembrane domain (TM), and the intraviral (or cytoplasmic) domain (cytodomain).

Native HCMV gBs from various strains are known. For example, at least sixty HCMV gB sequences from clinical and laboratory-adapted strains are available from NCBI's RefSeq database.

Accordingly, the term “CMV gB” polypeptide or “HCMV gB” polypeptide as used herein is to be understood as the native HCMV gB polypeptide from any human HCMV strain (not limited to the Towne strain). The actual residue position number may need to be adjusted for gBs from other human CMV strains depending on the actual sequence alignment.

HCMV gB is encoded by the UL55 gene of the HCMV genome. It is an envelope glycoprotein that mediates the fusion of the HCMV viral membrane with a host cell membrane. The protein undergoes a series of conformational changes from a prefusion to a postfusion form. The crystal structure of gB in its postfusion form is available (PDB accession codes 5CXF, 5C6T, and 7KDD), and a cryo-EM structure of the prefusion conformation has been recently determined (Liu et al., 2021; PDB accession code 7KDP).

An HCMV gB postfusion conformation refers to a structural conformation adopted by HCMV gB subsequent to the fusion of the virus envelope with the host cellular membrane. The native HCMV gB may also assume the postfusion conformation outside the context of a fusion event, for example, under stress conditions such as exposure to heat, extraction from a membrane, expression as an ectodomain, or storage. More specifically, the gB postfusion conformation is described, for example, in Burke et al., Crystal Structure of the Human Cytomegalovirus Glycoprotein B. PLoS Pathog. 2015 Oct. 20; 11(10): e1005227. See also, Research Collaboratory for Structural Bioinformatics Protein Data Bank (RCSB PDB): 5CXF, Crystal structure of the extracellular domain of glycoprotein B from Human Cytomegalovirus, from Human cytomegalovirus (strain AD169), deposited 2015-07-28; DOI: 10.2210/pdb5CXF/pdb. The postfusion conformation is about 165 Å tall and 65 Å wide.

As used herein, a “prefusion conformation” refers to a structural conformation adopted by the polypeptide that differs from the HCMV gB postfusion conformation at least in terms of molecular dimensions or three-dimensional coordinates. The prefusion conformation refers to a structural conformation adopted by HCMV gB prior to triggering of the fusogenic event that leads to transition of gB to the postfusion conformation. Isolating HCMV gB in a stable prefusion conformation may be useful in informing and directing development of improved vaccines and immunogenic compositions to address the important public health problem of cytomegalovirus infections. A prefusion conformation may be a conformation that can bind to a prefusion-specific antibody. A polypeptide having an HCMV gB prefusion conformation may refer to a polypeptide that includes a trimeric helix bundle, centered on the three-fold axis of the trimer and comprising residues L479 to K522 of each protomer, wherein the direction of the bundle from N-terminal to C-terminal along the three-fold axis is towards the point on the three-fold axis intersected by the plane defined by residue W240 of each protomer, which is in a fusion loop near the tip of each Domain I of the trimer.

### II. PROTEINS OF THE DISCLOSURE

The present disclosure provides engineered proteins that include amino acid mutations relative to the amino acid sequence of the corresponding wild-type HCMV gB. The amino acid mutations include amino acid substitutions, deletions, or additions relative to a wild-type HCMV gB. Accordingly, the engineered proteins are mutants of wild-type HCMV gBs.

The engineered proteins may possess certain beneficial characteristics, such as being immunogenic. The engineered proteins may possess increased immunogenic properties or improved stability in the prefusion conformation, as compared to the corresponding wild-type HCMV gB. Stability refers to the degree to which a transition of the HCMV gB conformation from prefusion to postfusion is hindered or prevented. The engineered proteins may display one or more introduced mutations as described herein, which may also result in improved stability in the prefusion conformation. The introduced amino acid mutations in the HCMV gB include amino acid substitutions, deletions, and/or additions. The mutations in the amino acid sequences of the engineered proteins may be amino acid substitutions, insertions, and/or deletions relative to a wild-type HCMV gB ectodomain.

Several modes of stabilizing the engineered protein conformation include amino acid substitutions that introduce disulfide bonds (both intraprotomer and interprotomer), modify salt bridges, introduce hydrophobic zippers (i.e., two cavity filling substitutions near each other that allow a third hydrophobic residue to insert between them), introduce electrostatic mutations, fill cavities, alter the packing of residues, cap helices, and combinations thereof, as compared to a native HCMV gB.

The engineered proteins may be isolated, i.e., separated from HCMV gB proteins having a postfusion conformation. Thus, the engineered proteins may be, for example, at least 80% isolated, at least 90% isolated, at least 95% isolated, at least 98% isolated, at least 99% isolated, or at least 99.9% isolated from HCMV gB polypeptides in a postfusion conformation. The engineered proteins may specifically bind to an HCMV gB prefusion-specific antibody.

It will be understood that a homogeneous population of engineered proteins in a particular conformation can include variations (such as polypeptide modification variations, e.g., glycosylation state), that do not alter the conformational state of the engineered proteins. The population of engineered proteins may remain homogeneous over time. For example, the engineered proteins, when dissolved in aqueous solution, may form a population of proteins stabilized in the prefusion conformation for at least 12 hours, at least 24 hours, at least 48 hours, at least one week, at least two weeks, or more. A person of ordinary skill in the art will appreciate that the engineered proteins provided herein are useful to elicit immune responses in mammals to CMV.

The native HCMV gB is conserved among the HCMV entry glycoproteins and is required for entry into all cell types. In view of the substantial conservation of HCMV gB sequences, the amino acid positions amongst different native HCMV gB sequences may be compared to identify corresponding HCMV gB amino acid positions among different HCMV strains. Thus, the conservation of native HCMV gB sequences across strains allows use of a reference HCMV gB sequence for comparison of amino acids at particular positions in the HCMV gB polypeptide. Accordingly, unless expressly indicated otherwise, the polypeptide amino acid positions provided herein refer to the reference sequence of the HCMV gB polypeptide set forth in SEQ ID NO: 1.

However, it should be noted that different native HCMV gB sequences may have different numbering systems from SEQ ID NO: 1, for example, there may be additional amino acid residues added or removed as compared to SEQ ID NO: 1 in a native HCMV gB sequence derived from a strain other than Towne. As such, it is to be understood that when specific amino acid residues are referred to by their number, the description is not limited to only amino acids located at precisely that numbered position when counting from the beginning of a given amino acid sequence, but rather that the equivalent or corresponding amino acid residue in any and all HCMV gB sequences is intended even if that residue is not at the same precise numbered position, for example if the HCMV sequence is shorter or longer than SEQ ID NO: 1, or has insertions or deletions as compared to SEQ ID NO: 1.

The engineered HCMV gB protein may be a truncated polypeptide lacking one or more of the following domain sequences as compared to SEQ ID NO: 1: (1) MPR domain (residues 705-750), (2) TM domain (residues 751-772), or (3) the CT domain (residues 773-907). As used herein the term “truncated” shall mean that a sequence is missing some or all of the residues comprising a domain as set forth herein.

As described herein, CMV gB protein comprises the following domains and residues (SEQ ID NO:1): (i) Domain I (residues 133-340), (ii) Domain II (residues 121-132 and 341-436), (iii) Domain III (residues 105-120, 483-533), (iv) Domain IV (residues 74-104, 534-641), (v) Domain V (residues 642-704), (vi) membrane-proximal region (MPR) (residues 705-750), (vii) transmembrane domain (TM) (residues 751-772), and (viii) cytoplasmic domain (CT) (residues 773-907). In another aspect, the ectodomain of CMV gB comprises residues 1-704 or 23-704 (without signal sequence) of SEQ ID NO: 1.

### A. Cysteine (C) Substitutions

The engineered proteins may include cysteine substitutions that are introduced, as compared to a native HCMV gB. The engineered protein may include any one of 1, 2, 3, 4, 5, 6, 7, 8, 9, or 10 cysteine substitutions. Without being bound by theory or mechanism, the cysteine substitutions described herein are believed to facilitate stability of the polypeptide in a conformation that is not the HCMV gB postfusion conformation. The introduced cysteine substitutions may be introduced by protein engineering, for example, by including one or more substituted cysteine residues that form a disulfide bond. The amino acid positions of the cysteines may be within a sufficiently close distance for formation of a disulfide bond in the prefusion, and not postfusion, conformation of the HCMV gB.

The cysteine residues that form a disulfide bond can be introduced into native HCMV gB sequence by two or more amino acid substitutions. For example, two cysteine residues may be introduced into a native HCMV gB sequence to form a disulfide bond.

The engineered proteins may include a recombinant HCMV gB stabilized in a prefusion conformation by a disulfide bond between cysteines that are introduced into a pair of amino acid positions that are close to each other in the prefusion conformation and more distant in the postfusion conformation. The pair of cysteines may both be present in a single protomer, thus forming an intraprotomer disulfide bond, or the pair of cysteines may be in different protomers, thus forming an interprotomer disulfide bond.

Exemplary cysteine substitutions as compared to a native HCMV gB include any disulfide bond substitutions in Table 1, the numbering of which is based on the numbering of SEQ ID NO: 1. The engineered proteins may include a combination of two or more of the disulfide bonds between paired cysteine residues listed in Table 1.

Amino acids may be inserted (or deleted) from the native HCMV gB sequence to adjust the alignment of residues in the polypeptide structure, such that particular residue pairs are within a sufficiently close distance to form a disulfide bond in the prefusion, but not postfusion, conformation. As such, the engineered proteins may include a disulfide bond between cysteine residues located at any of the pairs of positions listed in Table 1, in addition to including at least one amino acid insertion.

### B. Electrostatic Substitutions

The engineered proteins may include a phenylalanine substitution as compared to a native HCMV gB. The engineered protein may include a leucine substitution as compared to a native HCMV gB. The engineered protein may be stabilized by amino acid mutations (such as, for example, phenylalanine (F) and leucine (L) substitutions) that decrease ionic repulsion between residues that are proximate to each other in the folded structure of the protein in prefusion conformation, as compared to a HCMV gB polypeptide in postfusion conformation. The engineered proteins may be stabilized by amino acid mutations that increase ionic attraction between residues that are proximate to each other in the folded structure of the polypeptide in prefusion conformation, as compared to a HCMV gB in postfusion conformation.

Amino acids may be inserted (or deleted) from the native HCMV gB sequence to adjust the alignment of residues in the polypeptide structure, such that particular residue pairs are within a sufficiently close distance to form a desired electrostatic interaction in the prefusion, but not postfusion, conformation. As such, the engineered protein may include a desired electrostatic interaction at any of the positions listed in Table 1.

### C. Cavity Filling Mutations

The engineered proteins may include amino acid mutations that are one or more cavity filling mutations. Examples of amino acids that may be replaced with the goal of cavity filling include small aliphatic (e.g. Gly, Ala, and Val) or small polar amino acids (e.g. Ser and Thr) and amino acids that are buried in the pre-fusion conformation, but exposed to solvent in the post-fusion conformation. Examples of the replacement amino acids include large aliphatic amino acids (Ile, Leu and Met) or large aromatic amino acids (His, Phe, Tyr, and Trp).

### D. Combination of Mutations

The engineered ectodomains or engineered proteins may include a combination of two or more different types of mutations selected from engineered disulfide bond mutations, cavity filling mutations, and electrostatic mutations. The engineered ectodomains or engineered proteins may include at least one disulfide bond mutation and at least electrostatic mutation. The engineered ectodomains or engineered proteins may include at least one cysteine substitution and at least one cavity filling substitutions. The engineered ectodomains or engineered proteins may include at least one cysteine substitution and at least one charge reduction substitution. The engineered ectodomains or engineered proteins may include at least one mutation selected from any one of the mutations, or sets of mutations, in Table 1. Exemplary sets of mutations are provided in Table 2.

The substitutions in each of the constructs described in Tables 1 and 2 were introduced into a base construct having the amino acid sequence shown in SEQ ID NO: 4. The amino acid sequence of the base construct ectodomain only, i.e., without the signal sequence of any C-terminal modifications) is shown in SEQ ID NO: 5. The amino acid sequence of the base construct without the signal sequence by fused at the C-terminus to foldon trimerization motif of T4 fibritin (Fd) is shown in SEQ ID NO: 6. In the base construct, amino acids 23-704 of SEQ ID NO: 4 (or amino acids 1-682 of SEQ ID NO: 5) correspond to amino acids 23-704 of the HCMV Towne strain gB (SEQ ID NO: 1) with serine substituted at residues 246, 457 and 460 (relative to SEQ ID NO: 1) to remove an unpaired, non-conserved cysteine and the furin cleavage site (Burke et al., 2015; Nelson et al., 2020). A foldon trimerization motif of T4 fibritin (Fd) (SEQ ID NO: 6) is fused to the C-terminus of the gB ectodomain by a Gly-Ser linker in the base construct. The immature construct (i.e., SEQ ID NO: 4) further includes a signal sequence, an HRV3C protease recognition site, an octa-histidine tag, and a tandem Twin-Strep-tag.

In some constructs, the Fd was replaced by or combined with other trimerization motifs. These constructs are indicated in Tables 1 and 2 as having a C-terminal alteration. Constructs that are not listed as having a C-terminal alteration mutation type have the Fd of the base construct.

In constructs indicated as having GCN4 replacing C-Term, the Fd present in the base construct was replaced by a modified PIV5/GCN4 hybrid. Specifically, for the modified GCN4 constructs, the Fd trimerization motif was removed, along with residues 694-704 of gB (i.e., positions V694-P704 of SEQ ID NO: 4, or positions V672-P682 of SEQ ID NO: 6). In their place, the sequence 694-DTYLSAIEDKIEEILSKIYHIENEIARI-721 (SEQ ID NO: 83) was inserted. As an example, the amino acid sequence of the JSM-GCN4 construct is provided in SEQ ID NO: 61.

In constructs indicated as having 4J4A replacing C-Term, the Fd present in the base construct was replaced by a modified modified Cortexillin C-term (4J4A). Specifically, for the modified 4J4A constructs, the Fd trimerization motif was removed, along with residues 690-704 of gB (i.e., positions Y690-P704 of SEQ ID NO: 4, or positions Y668-P682 of SEQ ID NO: 6). In their place, the sequence 690-LKQIVLRIMEIEARIAKIE-708 (SEQ ID NO: 86) was inserted. As an example, the amino acid sequence of the JSM-4J4A construct is provided in SEQ ID NO: 62.

In constructs indicated as having a combined 4J4A+Fd C-terminus, the Fd trimerization motif was removed, along with residues 690-704 of gB (i.e., positions Y690-P704 of SEQ ID NO: 4, or positions Y668-P682 of SEQ ID NO: 6), and the sequence 690-LKQIVLRIMEIEARIAKIEGSGYIPEAPRDGQAYVRKDGEWVLLSTFLG-738 (SEQ ID NO: 87) was inserted in their place. As an example, the amino acid sequence of the gB-63 construct is provided in SEQ ID NO: 48.

TABLE 1
Engineered HCMV gB proteins (all positions relative to SEQ ID NO: 1 or SEQ ID NO: 4)

| Construct | | |
|---|---|---|
| ID | Mutation(s) | Type |
| CL-1 | L548C/P655C | Disulfide bond (inter) |
| CL-2 | I683P | Pro at beginning of helix |
| CL-3 | A650P/L651P | Pro at loop |
| CL-4 | K691P | Pro at the middle of a kink helix |
| CL-5 | E119P | Pro |
| CL-6 | L121P | Pro |
| CL-7 | D122P | Pro |
| CL-8 | E359R | Salt bridge with E119/D120 |
| CL-9 | S367C/A503C | Disulfide bond, anchor DII to DIII |
| CL-10 | Y153C/Y242C/W240A | reduce HQ, disulfide bond |
| CL-11 | Y153C/Y160C/W240A | reduce HQ, disulfide bond |
| CL-12 | Y155E/W240R | reduce HQ, salt bridge |
| CL-13 | I305E | Salt bridge with R141 |
| CL-14 | V645L | Cav |
| CL-15 | A396V | Cav |
| CL-16 | A500M | Cav |
| CL-17 | V127F | Cav |
| CL-18 | F541W | Cav |
| CL-19 | S641F | Cav |
| CL-20 | G433H | Cav, polar |
| CL-21 | A267M | Cav |
| CL-22 | I620F | Cav |
| CL-23 | T245E | Polar |
| CL-24 | S219C/N586C | Disulfide bond |
| CL-25 | 436-GGP-489 | Remove flexible region |
| CL-26 | 436-GPP-489 | Remove flexible region |
| CL-27 | 436-PGP-489 | Remove flexible region |
| CL-28 | I356F | Cav |
| CL-29 | D120R | Salt bridge with D489 |
| CL-30 | 437-CGADPQLC-486 | Based on JSM Cap1a |
| | (SEQ ID NO: 76) | |
| CL-31 | 437-SGCDPQLC-486 | Based on JSM Cap1a |
| | (SEQ ID NO: 77) | |
| CL-32 | A97C/R540C | Disulfide bond |
| CL-33/PB-3 | G543C/K617C | Disulfide bond (inter) |
| CL-34 | N750C/G753C | Disulfide bond, for MPR/TMD |
| CL-35 | T644C/S647C | Disulfide bond |
| CL-36 | Q483P | Pro |
| CL-37 | S367M | Cav |
| CL-38 | D630R | Salt bridge with 272/329 (inter) |
| CL-39 | P614R | Salt bridge with 272/329 (inter) |
| CL-40 | L616W | Cav |
| CL-41 | L102W | Cav |
| CL-42 | S641E | Polar |
| CL-43 | S526K | Salt bridge (inter), Cav |
| CL-44 | R354E | Salt bridge with R497 |
| CL-45 | D646W | Cav |
| CL-46 | D679C/E686C | Disulfide bond |
| CL-47 | S223V/T253V/T270M/T659V | Cav cluster |
| MS-1 | L241R/V728E | Salt bridge, anchor DI to MPR |
| MS-2 | W240R/A732E | Salt bridge, anchor DI to MPR |
| MS-3 | L162C/M716C | Salt bridge, anchor DI to MPR |
| MS-4 | Y160C/LPP-Y708C | Disulfide bond, anchor DI to MPR |
| MS-5 | Y153R/Y712E | Salt bridge, anchor DI to MPR |
| MS-6 | Y242C/G731C | Disulfide bond, anchor DI to MPR |
| MS-7 | M126C/W431C/R357W | Disulfide bond |
| MS-8 | V128C/V429C | Disulfide bond |
| MS-9 | F421C/V429C | Disulfide bond |
| MS-10 | Y415C/G433C | Disulfide bond |
| MS-11 | Y415C/K435C | Disulfide bond |
| MS-12 | Y155E/G735K | Salt bridge, anchor DI to MPR |
| MS-13 | W240C/G735C | Disulfide bond |
| MS-14 | Y242D/L715K | Salt bridge, anchor DI to MPR |
| MS-15 | V733D/F752K | Salt bridge, lock MPR |
| MS-16 | N750K | Salt bridge with D714, lock MPR |
| MS-17 | K710E | Salt bridge with K749, lock MPR, otherwise +/+ repulsion |
| MS-18 | I356C/A500C | Disulfide bond, anchor DII to DIII |
| MS-19 | Y129P | Pro at end of beta sheet, removes h-bond in post-fusion |
| MS-20 | Y155E/L709K | Salt bridge, anchor DI to MPR |
| MS-21 | Y168E | Salt bridge with R662, anchor DI to DV |
| MS-22 | I653D | Salt bridge with R258, anchor DI to DV |
| MS-23 | I103R | Salt bridge with D652, anchor DI to DV |
| MS-24 | K340P | Pro in middle of little helix |
| MS-25 | N341P | Pro in middle of little helix |
| MS-26 | S219C/A585C | Disulfide bond, anchor DI to DIV |
| MS-27 | Y218D/F584K | Salt bridge, anchor DI to DIV |
| MS-28 | V93C/L603C | Disulfide bond, anchor DI to DIV |
| MS-29 | H222C/E657C | Disulfide bond, anchor DI to DV |
| MS-30 | F198W | Steric hindrance in post-fusion |
| MS-31 | L161W | Steric hindrance in post-fusion |
| MS-32 | T159W | Cav |
| MS-33 | A97C/A549C | Disulfide bond, anchor DI to DIV |
| MS-34 | L427P | Pro at end of beta sheet |
| MS-35 | A351P | Pro at end of beta sheet |
| MS-36 | A482P | Pro at end of helix |
| MS-37 | A650D | Salt bridge with K130, anchor DI to DV |
| MS-38 | A257P | Pro in a beta sheet |
| MS-39 | L656D | Salt bridge with R258, anchor DI to DV |
| MS-40 | A373P | Pro at end of beta sheet |
| MS-41 | G426P | Pro at end of beta sheet |
| MS-42 | A373F | Steric hindrance in post-fusion |
| MS-43 | L484P | Pro at end of helix |
| MS-44 | Q501E | Salt bridge with R354, anchor DII to DIII |
| MS-45 | D654E | Salt bridge with K535, anchor DIV to DV |
| MS-46 | L613D | Salt bridge with R662, anchor DIV to DV |
| MS-47 | Y592E | Salt bridge with K670, anchor DIV to DV |
| MS-48 | V702E | Salt bridge with R673, inter-DV |
| MS-49 | E671P/L672P | Pro in middle of helix |
| MS-50 | S674C/E698C | Interprotomer disulfide bond |
| MS-51 | M684P | Pro at end of helix |
| MS-52 | D679C/R685C | Disulfide bond |
| MS-53 | E686Q | Eliminate −/− repulsion |
| MS-54 | G711L | Cav |
| MS-55 | L715F | Cav, pseudo inhibitor |
| MS-56 | G731M | Cav |
| MS-57 | A738M | Cav |
| MS-58 | V741Y | Cav, pseudo inhibitor |
| MS-55, 58 | L715F, V741Y, N750W | Cav |
| MS-59 | A725P, V726P, A727P | Pro scan at beginning of helix |
| MS-60 | G720P, A721P, A722P | Pro scan at end of helix |
| MS-61 | D509L | Remove calcium ion from central helices (eliminate |
| | | charge/charge repulsion/interaction) |
| MS-62 | R511Q | Eliminate charge |
| MS-63 | R512Q | Eliminate charge |
| MS-64 | V110E | Salt bridge with R511 |
| MS-65 | V93C/V552C | Disulfide bond, anchor DI to DIV |
| MS-66 | K749E | Salt bridge, eliminate +/+ repulsion |
| MS-67 | R497E | Salt bridge with R354, eliminates +/+ repulsion, |
| | | anchors DII to DIII |
| MS-68 | L121C/N496C | Disulfide bond, anchor DI to DIII |
| MS-69 | E167I | Stabilize pre-fusion, break salt bridge in post- |
| | | fusion |
| MS-70 | Y153F | Stabilize pre-fusion |
| MS-71 | Q612C/R662C | Disulfide bond, anchor DIV to DV |
| MS-72 | N605E | Salt bridge with K670, anchor DIV to DV |
| MS-73 | E686L/K691M | Charge reduction to Cav |
| MS-74 | R685L/R607M | Charge reduction to Cav |
| MS-75 | D679V/E682N | Charge reduction to Cav |
| MS-76 | R354W | Charge reduction to Cav |
| MS-77 | L162W | Steric hindrance in post-fusion, cav in pre-fusion |
| MS-78 | R662W | Cavity filling at interface |
| MS-79 | I103Y | Cavity filling at interface |
| MS-80 | Q98L/N658I | Hydrophobic zipper |
| MS-81 | R512W | Pi-pi interactions, eliminate charge |
| MS-82 | L161R | Help solubility |
| MS-83 | E686P | Pro |
| MS-84 | E686M | Cav |
| MS-85 | Y89C/N599C | Disulfide bond |
| JSM-1 | K700V | Eliminate charge and pack |
| JSM-2 | R693L | Eliminate charge and pack |
| JSM-1 + 2 | R693L/K700V | Eliminate charge and pack |
| JSM-3 | E686N | Eliminate charge and hydrogen bond |
| JSM-4 | H477P | Pro |
| JSM-5 | N478P | Pro |
| JSM-6 | L479P | Pro |
| JSM-7 | V480P | Pro |
| JSM-8 | Y481P | Pro |
| JSM-9 | E106C/T644C/R104I | Disulfide bond and charge reduction |
| JSM-10 | V645P | Pro at C-terminal hinge |
| JSM-11 | D646P | Pro at C-terminal hinge |
| JSM-12 | T100L/A267I | Hydrophobic zipper |
| JSM-13 | T659V/N220F | Hydrophobic zipper |
| JSM-14 | T221C/T553C | Disulfide bond |
| JSM-15 | L664F | Cav at interface |
| JSM-16 | T253L/T270L | Hydrophobic zipper |
| JSM-17 | L161E | Help solubility |
| JSM-18 | S689Y | Cav near trimer |
| JSM-19 | R607Y | Cav and charge reduction |
| JSM-20 | V273K | Salt bridge |
| JSM-21 | V273F | Cav |
| JSM-22 | A500L/A503S | Cav and hydrogen bond |
| JSM-23 | T270W | Cav |
| Apx1 | S2PA | Apex |
| Apx2 | Cap1a | Apex |
| Apx3 | Cap1b | Apex |
| Apx4 | Cap1eg2 DS | Apex |
| Apx5 | Cap1xvw PPP | Apex |
| JSM-GCN4 | GCN4 replacing C-Term | C-terminal alteration |
| JSM-4J4A | 4J4A replacing C-Term | C-terminal alteration |
| JG-1 | D509A | Ablate Ca2+ site/destabilize post-fusion |
| JG-2 | D509R | Ablate Ca2+ site/destabilize post-fusion |
| JG-3 | K130Y | Charge reduction to Cav |
| JG-4 | K130L | Charge reduction to Cav |
| JG-5 | M648E | Salt bridge |
| JG-6 | T112L | Cav |
| JG-7 | T112Y | Cav |
| JG-8 | K260W | Cav |
| JG-9 | T253L | Cav |
| JG-10 | S643W | Cav |
| JG-11 | S647L | Cav |
| JG-12 | N599Y | Cav |
| JG-13 | N556Y | Cav |
| JG-14 | E597F | Cav |
| JG-15 | E597Y | Cav |
| JG-16 | E627F | Cav |
| JG-17 | H606E | Salt bridge |
| JG-18 | K130W | Cav |
| RMc-1 | 436-GPG-489 | Remove flexible region |
| RMc-2 | Y160C/W240A/Y708C | Disulfide bond, replaced exposed hydrophobic residue within |
| | | the fusion loops with more hydrophilic HSV-1 counterpart |
| DW-1 | D217C/S587C | Disulfide bond for flexible regions of DI/DIV |
| DW-2 | Q212W | Cav between two loops |
| DW-3 | L248I | Cav |
| DW-4 | Y261P | Pro in loop |
| DW-5 | I305M | Cav |
| DW-6 | L346Y | Pi-pi interactions to disrupt postfusion betafold |
| DW-7 | Y415N | Salt bridge with K435 |
| DW-8 | F421H | His in beta barrel |
| DW-9 | G433P | Pro at end of beta sheet |
| DW-10 | G493M | Cav, DI/DIII hydrophobic interface |
| DW-11 | R497Q | Remove postfusion DIII/DV salt bridge, remove +/+ repulsion |
| | | in prefusion, results in −/− charge repulsion in postfusion |
| DW-12 | F517W | Cav |
| DW-13 | L546I | Cav |
| DW-14 | G547A | Cav |
| DW-15 | N566H | Pi-pi interactions |
| DW-16 | S624K | Salt bridge with D638 |
| DT-7 | T347C/N132C | Disulfide bond |
| DT-8 | Q98C/G271C | Disulfide bond |
| DT-9 | G99C/G547C | Disulfide bond |
| DT-10 | A97C/G547C | Disulfide bond |
| HGB-1 | Y155G | Replaced exposed hydrophobic residue within the |
| | | fusion loops with more hydrophilic HSV-1 counterpart |
| HGB-2 | I156H | Replaced exposed hydrophobic residue within the |
| | | fusion loops with more hydrophilic HSV-1 counterpart |
| HGB-3 | Y157R | Replaced exposed hydrophobic residue within the |
| | | fusion loops with more hydrophilic HSV-1 counterpart |
| HGB-4 | W240A | Replaced exposed hydrophobic residue within the |
| | | fusion loops with more hydrophilic HSV-1 counterpart |
| HGB-5 | L241T | Replaced exposed hydrophobic residue within the |
| | | fusion loops with more hydrophilic EBV counterpart |
| HGB-6 | Y242H | Replaced exposed hydrophobic residue within the |
| | | fusion loops with more hydrophilic HSV-1 counterpart |
| LZ-1 | D272N | Disrupts post-fusion salt bridge |
| LZ-2 | del Y189-Y201 | Deletes unstructured pre-fusion linker/structured |
| | | post-fusion |
| LZ-3 | del Y280-A294 | Shortens linker in pre-fusion |
| LZ-4 | del F265-N284 | Deletes flexible linker that closely interacts |
| | | with V helix in post-fusion |
| PB-1 | G99C/L616C | Interprotomer disulfide bond |
| PB-2 | G543C/F619C | Interprotomer disulfide bond |
| PB-4 | Y89C/G622C | Interprotomer disulfide bond |
| PB-5 | G604C/Y667C | Interprotomer disulfide bond |
| PB-6 | M371C/A505C | Interprotomer disulfide bond |
| PB-8 | D544C/I620C | Interprotomer disulfide bond |
| PB-9 | D544C/Q597C | Interprotomer disulfide bond |
| PB-10 | E106C/T644C | Interprotomer disulfide bond |
| PB-11 | R104C/T644C | Interprotomer disulfide bond |
| PB-12 | R104C/I642C | Interprotomer disulfide bond |
| PB-13 | V273C/L616C | Interprotomer disulfide bond |
| PB-14 | E698C/R673C | Interprotomer disulfide bond |
| PB-16 | F661C/Y218C | Interprotomer disulfide bond |
| PB-17 | K260C/S641C | Interprotomer disulfide bond |
| PB-18 | T347C/A650C | Interprotomer disulfide bond |
| PB-19 | V545C/I618C | Interprotomer disulfide bond |
| PB-20 | A135C/I653C | Interprotomer disulfide bond |
| PB-21 | V134C/I653C | Interprotomer disulfide bond |
| PB-22 | N220C/E657C | Interprotomer disulfide bond |
| PB-101 | G271C/P614C | Interprotomer disulfide bond |
| PB-102 | G604C/L672C | Interprotomer disulfide bond |
| PB-103 | S219C/A585C | Interprotomer disulfide bond |
| PB-104 | W349C/A650C | Interprotomer disulfide bond |
| PB-105 | A549C/N658C | Interprotomer disulfide bond |
| PB-106 | Q98C/N658C | Interprotomer disulfide bond |
| PB-107 | S550C/E657C | Interprotomer disulfide bond |
| PB-108 | S550C/P655C | Interprotomer disulfide bond |

TABLE 2
Exemplary combinations of substitutions (all positions
relative to SEQ ID NO: 1 or SEQ ID NO: 4)

| Construct | Combined | | |
|---|---|---|---|
| ID | Constructs | Mutation(s) | Type |
| gB-1 | JSM-10 | V645P | Pro at C-terminal hinge |
| | CL-41 | L102W | Cav |
| | JSM-1 + 2 | R693L, K700V | Eliminate charge and pack |
| gB-2 | JSM-10 | V645P | Pro at C-terminal hinge |
| | MS-73 | E686L | Charge reduction to Cav |
| | MS-29 | H222C/E657C | Disulfide bond, anchor DI to DV |
| gB-3 | JSM-9 | E106C/T644C, R104I | Disulfide bond and charge reduction |
| | JSM-12 | A267I, T100L | Hydrophobic zipper |
| | JSM-1 + 2 | R693L, K700V | Eliminate charge and pack |
| gB-4 | JSM-9 | E106C/T644C, R104I | Disulfide bond and charge reduction |
| | CL-41 | L102W | Cav |
| | MS-29 | H222C/E657C | Disulfide bond, anchor DI to DV |
| gB-5 | JG-3 | K130Y | Charge reduction to Cav |
| | JSM-12 | A267I, T100L | Hydrophobic zipper |
| | JSM-1 + 2 | R693L, K700V | Eliminate charge and pack |
| gB-6 | JG-3 | K130Y | Charge reduction to Cav |
| | MS-73 | E686L | Charge reduction to Cav |
| | MS-29 | H222C/E657C | Disulfide bond, anchor DI to DV |
| gB-7 | Pf-70 (CL-9) | A503C/S367C | Disulfide bond, anchor DII to DIII |
| | CL-41 | L102W | Cav |
| | JSM-1 + 2 | R693L, K700V | Eliminate charge and pack |
| gB-8 | Pf-70 (CL-9) | A503C/S367C | Disulfide bond, anchor DII to DIII |
| | JSM-21 | V273F | Cav |
| | MS-29 | H222C/E657C | Disulfide bond, anchor DI to DV |
| gB-9 | MS-18 | I356C/A500C | Disulfide bond, anchor DII to DIII |
| | JSM-12 | A267I, T100L | Hydrophobic zipper |
| | JSM-1 + 2 | R693L, K700V | Eliminate charge and pack |
| gB-10 | MS-29 | H222C/E657C | Disulfide bond, anchor DI to DV |
| | MS-50 | S674C/E698C | Interprotomer disulfide bond |
| gB-11 | PB-22 | N220C/E657C | Interprotomer disulfide bond |
| | MS-50 | S674C/E698C | Interprotomer disulfide bond |
| gB-12 | MS-29 | H222C/E657C | Disulfide bond, anchor DI to DV |
| | JSM-12 | T100L/A267I | Hydrophobic zipper |
| gB-13 | PB-22 | N220C/E657C | Interprotomer disulfide bond |
| | JSM-12 | T100L/A267I | Hydrophobic zipper |
| gB-14 | MS-29 | H222C/E657C | Disulfide bond, anchor DI to DV |
| | JG-8 | K260W | Cav |
| gB-15 | PB-22 | N220C/E657C | Interprotomer disulfide bond |
| | JG-8 | K260W | Cav |
| gB-16 | MS-29 | H222C/E657C | Disulfide bond, anchor DI to DV |
| | JSM-10 | V645P | Pro at C-terminal hinge |
| gB-17 | PB-22 | N220C/E657C | Interprotomer disulfide bond |
| | JSM-10 | V645P | Pro at C-terminal hinge |
| gB-18 | MS-29 | H222C/E657C | Disulfide bond, anchor DI to DV |
| | MS-69 | E167I | Stabilize pre-fusion, break salt bridge in post-fusion |
| gB-19 | PB-22 | N220C/E657C | Interprotomer disulfide bond |
| | MS-69 | E167I | Stabilize pre-fusion, break salt bridge in post-fusion |
| gB-20 | MS-29 | H222C/E657C | Disulfide bond, anchor DI to DV |
| | JSM-21 | V273F | Cav |
| gB-21 | PB-22 | N220C/E657C | Interprotomer disulfide bond |
| | JSM-21 | V273F | Cav |
| gB-22 | MS-29 | H222C/E657C | Disulfide bond, anchor DI to DV |
| | CL-41 | L102W | Cav |
| gB-23 | PB-22 | N220C/E657C | Interprotomer disulfide bond |
| | CL-41 | L102W | Cav |
| gB-24 | MS-29 | H222C/E657C | Disulfide bond, anchor DI to DV |
| | MS-80 | Q98L/N658I | Hydrophobic zipper |
| gB-25 | PB-22 | N220C/E657C | Interprotomer disulfide bond |
| | MS-80 | Q98L/N658I | Hydrophobic zipper |
| gB-26 | MS-29 | H222C/E657C | Disulfide bond, anchor DI to DV |
| | JG-3 | K130Y | Charge reduction to Cav |
| gB-27 | PB-22 | N220C/E657C | Interprotomer disulfide bond |
| | JG-3 | K130Y | Charge reduction to Cav |
| gB-28 | MS-29 | H222C/E657C | Disulfide bond, anchor DI to DV |
| | MS-25 | N341P | Pro in middle of little helix |
| gB-29 | PB-22 | N220C/E657C | Interprotomer disulfide bond |
| | MS-25 | N341P | Pro in middle of little helix |
| gB-30 | MS-29 | H222C/E657C | Disulfide bond, anchor DI to DV |
| | JSM1 | R693L | Eliminate charge and pack |
| | JSM2 | K700V | Eliminate charge and pack |
| gB-31 | PB-22 | N220C/E657C | Interprotomer disulfide bond |
| | JSM1 | R693L | Eliminate charge and pack |
| | JSM2 | K700V | Eliminate charge and pack |
| gB-32 | MS-29 | H222C/E657C | Disulfide bond, anchor DI to DV |
| | MS-77 | L162W | Steric hindrance in post-fusion, cav in pre-fusion |
| gB-33 | PB-22 | N220C/E657C | Interprotomer disulfide bond |
| | MS-77 | L162W | Steric hindrance in post-fusion, cav in pre-fusion |
| gB-34 | MS-29 | H222C/E657C | Disulfide bond, anchor DI to DV |
| | JSM-7 | V480P | Pro |
| gB-35 | PB-22 | N220C/E657C | Interprotomer disulfide bond |
| | JSM-7 | V480P | Pro |
| gB-36 | MS-29 | H222C/E657C | Disulfide bond, anchor DI to DV |
| | MS-43 | L484P | Pro at end of helix |
| gB-37 | PB-22 | N220C/E657C | Interprotomer disulfide bond |
| | MS-43 | L484P | Pro at end of helix |
| gB-38 | MS-29 | H222C/E657C | Disulfide bond, anchor DI to DV |
| | JSM-6 | L479P | Pro |
| gB-39 | PB-22 | N220C/E657C | Interprotomer disulfide bond |
| | JSM-6 | L479P | Pro |
| gB-40 | MS-29 | H222C/E657C | Disulfide bond, anchor DI to DV |
| | JSM-8 | Y481P | Pro |
| gB-41 | PB-22 | N220C/E657C | Interprotomer disulfide bond |
| | JSM-8 | Y481P | Pro |
| gB-42 | MS-29 | H222C/E657C | Disulfide bond, anchor DI to DV |
| | MS-71 | Q612C/R662C | Disulfide bond, anchor DIV to DV |
| gB-43 | PB-22 | N220C/E657C | Interprotomer disulfide bond |
| | MS-71 | Q612C/R662C | Disulfide bond, anchor DIV to DV |
| gB-44 | MS-29 | H222C/E657C | Disulfide bond, anchor DI to DV |
| | PB-6 | M371C/A505C | Interprotomer disulfide bond |
| gB-45 | PB-22 | N220C/E657C | Interprotomer disulfide bond |
| | PB-6 | M371C/A505C | Interprotomer disulfide bond |
| gB-46 | MS-29 | H222C/E657C | Disulfide bond, anchor DI to DV |
| | PB-17 | K260C/S641C | Interprotomer disulfide bond |
| gB-47 | PB-22 | N220C/E657C | Interprotomer disulfide bond |
| | PB-17 | K260C/S641C | Interprotomer disulfide bond |
| gB-48 | MS-29 | H222C/E657C | Disulfide bond, anchor DI to DV |
| | MS-18 | I356C/A500C | Disulfide bond, anchor DII to DIII |
| gB-49 | PB-22 | N220C/E657C | Interprotomer disulfide bond |
| | MS-18 | I356C/A500C | Disulfide bond, anchor DII to DIII |
| gB-50 | MS-29 | H222C/E657C | Disulfide bond, anchor DI to DV |
| | MS-4 | Y160C/LPP-Y708C | Salt bridge, anchor DI to MPR |
| gB-51 | PB-22 | N220C/E657C | Interprotomer disulfide bond |
| | MS-4 | Y160C/LPP-Y708C | Salt bridge, anchor DI to MPR |
| gB-53 | PB-22 | N220C/E657C | Interprotomer disulfide bond |
| | PB-21 | V134C/I653C | Interprotomer disulfide bond |
| gB-54 | PB-21 | V134C/I653C | Interprotomer disulfide bond |
| | PB-17 | K260C/S641C | Interprotomer disulfide bond |
| gB-55 | PB-21 | V134C/I653C | Interprotomer disulfide bond |
| | MS-71 | Q612C/R662C | Disulfide bond, anchor DIV to DV |
| gB-56 | PB-21 | V134C/I653C | Interprotomer disulfide bond |
| | MS-50 | S674C/E698C | Interprotomer disulfide bond |
| gB-57 | PB-21 | V134C/I653C | Interprotomer disulfide bond |
| | PB-6 | M371C/A505C | Interprotomer disulfide bond |
| gB-58 | PB-21 | V134C/I653C | Interprotomer disulfide bond |
| | MS-18 | I356C/A500C | Disulfide bond, anchor DII to DIII |
| gB-59 | PB-21 | V134C/I653C | Interprotomer disulfide bond |
| | MS-4 | Y160C/LPP-Y708C | Salt bridge, anchor DI to MPR |
| gB-60 | JSM-12 | T100L, A267I | Hydrophobic zipper |
| | 4J4A + Fd | | C-terminal alteration |
| gB-61 | MS-29 | H222C/E657C | Disulfide bond, anchor DI to DV |
| | 4J4A + Fd | | C-terminal alteration |
| gB-62 | PB-21 | V134C/I653C | Interprotomer disulfide bond |
| | 4J4A + Fd | | C-terminal alteration |
| gB-63 | PB-22 | N220C/E657C | Interprotomer disulfide bond |
| | 4J4A + Fd | | C-terminal alteration |
| gB-64 | PB-22 | N220C/E657C | Interprotomer disulfide bond |
| | MS-25 | N341P | Pro in middle of little helix |
| | MS-43 | L484P | Pro at end of helix |
| | JSM-10 | V645P | Pro at C-terminal hinge |
| gB-65 | PB-22 | N220C/E657C | Interprotomer disulfide bond |
| | JSM-12 | T100L/A267I | Hydrophobic zipper |
| | MS-80 | Q98L/N658I | Hydrophobic zipper |
| gB-66 | PB-22 | N220C/E657C | Interprotomer disulfide bond |
| | JSM-1 + 2 | R693L/K700V | Eliminate charge and pack |
| | MS-73 | E686L/K691M | Charge reduction to Cav |
| gB-67 | PB-22 | N220C/E657C | Interprotomer disulfide bond |
| | CL-41 | L102W | Cav |
| | JG-3 | K130Y | Charge reduction to Cav |
| | MS-77 | L162W | Steric hindrance in post-fusion, cav in pre-fusion |
| | JG-8 | K260W | Cav |
| | JSM-21 | V273F | Cav |
| gB-68 | PB-21 | V134C/I653C | Interprotomer disulfide bond |
| | MS-25 | N341P | Pro in middle of little helix |
| | MS-43 | L484P | Pro at end of helix |
| | JSM-10 | V645P | Pro at C-terminal hinge |
| gB-69 | PB-21 | V134C/I653C | Interprotomer disulfide bond |
| | JSM-12 | T100L/A267I | Hydrophobic zipper |
| | MS-80 | Q98L/N658I | Hydrophobic zipper |
| gB-70 | PB-21 | V134C/I653C | Interprotomer disulfide bond |
| | JSM-1 + 2 | R693L/K700V | Eliminate charge and pack |
| | MS-73 | E686L/K691M | Charge reduction to Cav |
| gB-71 | PB-21 | V134C/I653C | Interprotomer disulfide bond |
| | CL-41 | L102W | Cav |
| | JG-3 | K130Y | Charge reduction to Cav |
| | MS-77 | L162W | Steric hindrance in post-fusion, cav in pre-fusion |
| | JG-8 | K260W | Cav |
| | JSM-21 | V273F | Cav |
| gB-72 | PB-21 | V134C/I653C | Interprotomer disulfide bond |
| | JG-10 | S643W | Cav |
| gB-73 | PB-21 | V134C/I653C | Interprotomer disulfide bond |
| | JG-11 | S647L | Cav |
| gB-74 | PB-21 | V134C/I653C | Interprotomer disulfide bond |
| | JG-12 | N599Y | Cav |
| gB-75 | PB-21 | V134C/I653C | Interprotomer disulfide bond |
| | JG-13 | N566Y | Cav |
| gB-76 | PB-21 | V134C/I653C | Interprotomer disulfide bond |
| | JG-14 | E597F | Cav |
| gB-77 | PB-21 | V134C/I653C | Interprotomer disulfide bond |
| | JG-15 | E597Y | Cav |
| gB-78 | PB-21 | V134C/I653C | Interprotomer disulfide bond |
| | JG-16 | E627F | Cav |
| gB-79 | PB-21 | V134C/I653C | Interprotomer disulfide bond |
| | JG-17 | H606E | Salt bridge |
| gB-100 | PB-21 | V134C/I653C | Interprotomer disulfide bond |
| (gB-52) | MS-29 | H222C/E657C | Disulfide bond, anchor DI to DV |
| gB-107 | JSM-12 | T100L/A267I | Hydrophobic zipper |
| | PB-21 | V134C/I653C | Interprotomer disulfide bond |
| | MS-29 | H222C/E657C | Disulfide bond, anchor DI to DV |
| Apex2-1 | Apx2 | Cap1a | Apex |
| | 4J4A | 4J4A replacing | C-terminal alteration |
| | | C-Term | |
| Apex2-2 | Apx2 | Cap1a | Apex |
| | GCN4 | GCN4 replacing | C-terminal alteration |
| | | C-Term | |
| Apex2-3 | Apx2 | Cap1a | Apex |
| | JSM-12 | T100L/A267I | Hydrophobic zipper |
| | 4J4A | 4J4A replacing | C-terminal alteration |
| | | C-Term | |
| Apex4-1 | Apx4 | Cap1eg2 DS | Apex |
| | 4J4A | 4J4A replacing | C-terminal alteration |
| | | C-Term | |
| Apex 4-2 | Apx4 | Cap1eg2 DS | Apex |
| | GCN4 | GCN4 replacing | C-terminal alteration |
| | | C-Term | |
| Apex 4-3 | Apx4 | Cap1eg2 DS | Apex |
| | JSM-12 | T100L/A267I | Hydrophobic zipper |
| | 4J4A | 4J4A replacing | C-terminal alteration |
| | | C-Term | |
| Apex5-1 | Apx5 | Cap1xvw PPP | Apex |
| | 4J4A | 4J4A replacing | C-terminal alteration |
| | | C-Term | |
| Apex5-2 | Apx5 | Cap1xvw PPP | Apex |
| | GCN4 | GCN4 replacing | C-terminal alteration |
| | | C-Term | |
| Apex5-3 | Apx5 | Cap1xvw PPP | Apex |
| | JSM-12 | T100L/A267I | Hydrophobic zipper |
| | 4J4A | 4J4A replacing | C-terminal alteration |
| | | C-Term | |

In some embodiments, the engineered HCMV gB ectodomain protein includes positions 23-704 of SEQ ID NO: 4 and the amino acid substitutions 220C (such as N220C) and 657C (such as E657C). The engineered HCMV gB ectodomain protein may further include C246S, R457S, and R460S substitutions. The engineered HCMV gB ectodomain protein may further include linkage to a Fd trimerization domain (for example, via a GS peptide linker). The engineered HCMV gB ectodomain protein may comprise the amino acid sequence of SEQ ID NOs: 26 or 27, or an amino acid sequence at least 95%, at least 96%, at least 97%, at least 98%, or at least 99% identical to the amino acid sequence of SEQ ID NOs: 26 or 27.

In some embodiments, the engineered HCMV gB ectodomain protein includes positions 23-704 of SEQ ID NO: 4 and the amino acid substitution 260W (such as K260W). The engineered HCMV gB ectodomain protein may further include C246S, R457S, and R460S substitutions. The engineered HCMV gB ectodomain protein may further include linkage to a Fd trimerization domain (for example, via a GS peptide linker). The engineered HCMV gB ectodomain protein may comprise the amino acid sequence of SEQ ID NOs: 23 or 24, or an amino acid sequence at least 95%, at least 96%, at least 97%, at least 98%, or at least 99% identical to the amino acid sequence of SEQ ID NOs: 23 or 24.

In some embodiments, the engineered HCMV gB ectodomain protein includes positions 23-704 of SEQ ID NO: 4 and the amino acid substitution 273F (such as V273F). The engineered HCMV gB ectodomain protein may further include C246S, R457S, and R460S substitutions. The engineered HCMV gB ectodomain protein may further include linkage to a Fd trimerization domain (for example, via a GS peptide linker). The engineered HCMV gB ectodomain protein may comprise the amino acid sequence of SEQ ID NOs: 20 or 21, or an amino acid sequence at least 95%, at least 96%, at least 97%, at least 98%, or at least 99% identical to the amino acid sequence of SEQ ID NOs: 20 or 21.

In some embodiments, the engineered HCMV gB ectodomain protein includes positions 23-704 of SEQ ID NO: 4 and the amino acid substitution 130Y (such as K130Y). The engineered HCMV gB ectodomain protein may further include C246S, R457S, and R460S substitutions. The engineered HCMV gB ectodomain protein may further include linkage to a Fd trimerization domain (for example, via a GS peptide linker). The engineered HCMV gB ectodomain protein may comprise the amino acid sequence of SEQ ID NOs: 17 or 18, or an amino acid sequence at least 95%, at least 96%, at least 97%, at least 98%, or at least 99% identical to the amino acid sequence of SEQ ID NOs: 17 or 18.

In some embodiments, the engineered HCMV gB ectodomain protein includes positions 23-704 of SEQ ID NO: 4 and the amino acid substitutions 222C (such as H222C) and 657C (such as E657C). The engineered HCMV gB ectodomain protein may further include C246S, R457S, and R460S substitutions. The engineered HCMV gB ectodomain protein may further include linkage to a Fd trimerization domain (for example, via a GS peptide linker). The engineered HCMV gB ectodomain protein may comprise the amino acid sequence of SEQ ID NOs: 14 or 15, or an amino acid sequence at least 95%, at least 96%, at least 97%, at least 98%, or at least 99% identical to the amino acid sequence of SEQ ID NOs: 14 or 15.

In some embodiments, the engineered HCMV gB ectodomain protein includes positions 23-704 of SEQ ID NO: 4 and the amino acid substitutions 134C (such as V134C) and 653C (such as I653C). The engineered HCMV gB ectodomain protein may further include C246S, R457S, and R460S substitutions. The engineered HCMV gB ectodomain protein may further include linkage to a Fd trimerization domain (for example, via a GS peptide linker). The engineered HCMV gB ectodomain protein may comprise the amino acid sequence of SEQ ID NOs: 11 or 12, or an amino acid sequence at least 95%, at least 96%, at least 97%, at least 98%, or at least 99% identical to the amino acid sequence of SEQ ID NOs: 11 or 12.

In some embodiments, the engineered HCMV gB ectodomain protein includes positions 23-704 of SEQ ID NO: 4 and the amino acid substitutions 100L (such as T100L) and 267I (such as A267I). The engineered HCMV gB ectodomain protein may further include C246S, R457S, and R460S substitutions. The engineered HCMV gB ectodomain protein may further include linkage to a Fd trimerization domain (for example, via a GS peptide linker). The engineered HCMV gB ectodomain protein may comprise the amino acid sequence of SEQ ID NOs: 8 or 9, or an amino acid sequence at least 95%, at least 96%, at least 97%, at least 98%, or at least 99% identical to the amino acid sequence of SEQ ID NOs: 8 or 9.

In some embodiments, the engineered HCMV gB ectodomain protein includes positions 23-704 of SEQ ID NO: 4 and the amino acid substitutions 100L (such as T100L), 267I (such as A267I), 134C (such as V134C), 653C (such as I653C), 222C (such as H222C), and 657C (such as E657C). The engineered HCMV gB ectodomain protein may further include C246S, R457S, and R460S substitutions. The engineered HCMV gB ectodomain protein may further include linkage to a Fd trimerization domain (for example, via a GS peptide linker). The engineered HCMV gB ectodomain protein may comprise the amino acid sequence of SEQ ID NOs: 54 or 55, or an amino acid sequence at least 95%, at least 96%, at least 97%, at least 98%, or at least 99% identical to the amino acid sequence of SEQ ID NOs: 54 or 55.

In some embodiments, the engineered HCMV gB ectodomain protein includes positions 23-704 of SEQ ID NO: 4 and the amino acid substitutions 134C (such as V134C), 653C (such as I653C), 222C (such as H222C), and 657C (such as E657C). The engineered HCMV gB ectodomain protein may further include C246S, R457S, and R460S substitutions. The engineered HCMV gB ectodomain protein may further include linkage to a Fd trimerization domain (for example, via a GS peptide linker). The engineered HCMV gB ectodomain protein may comprise the amino acid sequence of SEQ ID NOs: 51 or 52, or an amino acid sequence at least 95%, at least 96%, at least 97%, at least 98%, or at least 99% identical to the amino acid sequence of SEQ ID NOs: 51 or 52.

In some embodiments, the engineered HCMV gB ectodomain protein includes positions 23-704 of SEQ ID NO: 4 and the amino acid substitutions 100L (such as T100L), 267I (such as A267I), 222C (such as H222C), and 657C (such as E657C). The engineered HCMV gB ectodomain protein may further include C246S, R457S, and R460S substitutions. The engineered HCMV gB ectodomain protein may further include linkage to a Fd trimerization domain (for example, via a GS peptide linker). The engineered HCMV gB ectodomain protein may comprise the amino acid sequence of SEQ ID NOs: 29 or 30, or an amino acid sequence at least 95%, at least 96%, at least 97%, at least 98%, or at least 99% identical to the amino acid sequence of SEQ ID NOs: 29 or 30.

In some embodiments, the engineered HCMV gB ectodomain protein includes positions 23-704 of SEQ ID NO: 4 and the amino acid substitutions 653C (such as I653C), 134C (such as V134C), 220C (such as N220C), and 657C (such as E657C). The engineered HCMV gB ectodomain protein may further include C246S, R457S, and R460S substitutions. The engineered HCMV gB ectodomain protein may further include linkage to a Fd trimerization domain (for example, via a GS peptide linker). The engineered HCMV gB ectodomain protein may comprise the amino acid sequence of SEQ ID NOs: 44 or 45, or an amino acid sequence at least 95%, at least 96%, at least 97%, at least 98%, or at least 99% identical to the amino acid sequence of SEQ ID NOs: 44 or 45.

In some embodiments, the engineered HCMV gB ectodomain protein includes positions 23-704 of SEQ ID NO: 4 and the amino acid substitutions 130Y (such as K130Y), 220C (such as N220C), and 657C (such as E657C). The engineered HCMV gB ectodomain protein may further include C246S, R457S, and R460S substitutions. The engineered HCMV gB ectodomain protein may further include linkage to a Fd trimerization domain (for example, via a GS peptide linker). The engineered HCMV gB ectodomain protein may comprise the amino acid sequence of SEQ ID NOs: 41 or 42, or an amino acid sequence at least 95%, at least 96%, at least 97%, at least 98%, or at least 99% identical to the amino acid sequence of SEQ ID NOs: 41 or 42.

In some embodiments, the engineered HCMV gB ectodomain protein includes positions 23-704 of SEQ ID NO: 4 and the amino acid substitutions 273F (such as V273F), 222C (such as H222C), and 657C (such as E657C). The engineered HCMV gB ectodomain protein may further include C246S, R457S, and R460S substitutions. The engineered HCMV gB ectodomain protein may further include linkage to a Fd trimerization domain (for example, via a GS peptide linker). The engineered HCMV gB ectodomain protein may comprise the amino acid sequence of SEQ ID NOs: 38 or 39, or an amino acid sequence at least 95%, at least 96%, at least 97%, at least 98%, or at least 99% identical to the amino acid sequence of SEQ ID NOs: 38 or 39.

In some embodiments, the engineered HCMV gB ectodomain protein includes positions 23-704 of SEQ ID NO: 4 and the amino acid substitutions 260W (such as H260W), 222C (such as H222C), and 657C (such as E657C). The engineered HCMV gB ectodomain protein may further include C246S, R457S, and R460S substitutions. The engineered HCMV gB ectodomain protein may further include linkage to a Fd trimerization domain (for example, via a GS peptide linker). The engineered HCMV gB ectodomain protein may comprise the amino acid sequence of SEQ ID NOs: 35 or 36, or an amino acid sequence at least 95%, at least 96%, at least 97%, at least 98%, or at least 99% identical to the amino acid sequence of SEQ ID NOs: 35 or 36.

In some embodiments, the engineered HCMV gB ectodomain protein includes positions 23-704 of SEQ ID NO: 4 and the amino acid substitutions 100L (such as T100L), 267I (such as A267I), 220C (such as N220C), and 657C (such as E657C). The engineered HCMV gB ectodomain protein may further include C246S, R457S, and R460S substitutions. The engineered HCMV gB ectodomain protein may further include linkage to a Fd trimerization domain (for example, via a GS peptide linker). The engineered HCMV gB ectodomain protein may comprise the amino acid sequence of SEQ ID NOs: 32 or 33, or an amino acid sequence at least 95%, at least 96%, at least 97%, at least 98%, or at least 99% identical to the amino acid sequence of SEQ ID NOs: 32 or 33.

### III. PROTEIN PREPARATION

The protein described herein may be prepared by routine methods known in the art, such as by expression in a recombinant host system using a suitable vector. Suitable recombinant host cells include, for example, insect cells, mammalian cells, avian cells, bacteria, and yeast cells. Examples of suitable insect cells include, for example, Sf9 cells, Sf21 cells, Tn5 cells, Schneider S2 cells, and HIGH FIVE cells (a clonal isolate derived from the parental Trichoplusia ni BTI-TN-5B1-4 cell line). Examples of suitable mammalian cells include Chinese hamster ovary (CHO) cells, human embryonic kidney cells (HEK293 or Expi 293 cells, typically transformed by sheared adenovirus type 5 DNA), NIH-3T3 cells, 293-T cells, Vero cells, and HeLa cells. Suitable avian cells include, for example, chicken embryonic stem cells (e.g., EBx.®. cells), chicken embryonic fibroblasts, chicken embryonic germ cells, quail fibroblasts (e.g., ELL-O), and duck cells. Suitable insect cell expression systems, such as baculovirus-vectored systems, are known to those of skill in the art. Materials and methods for baculovirus/insect cell expression systems are commercially available in kit form. Avian cell expression systems are also known to those of skill in the art. Similarly, bacterial and mammalian cell expression systems are also known in the art.

A number of suitable vectors for expression of recombinant proteins in insect or mammalian cells are well-known and conventional in the art. Suitable vectors can contain a number of components, including, but not limited to one or more of the following: an origin of replication; a selectable marker gene; one or more expression control elements, such as a transcriptional control element (e.g., a promoter, an enhancer, a terminator), and/or one or more translation signals; and a signal sequence or leader sequence for targeting to the secretory pathway in a selected host cell (e.g., of mammalian origin or from a heterologous mammalian or non-mammalian species). For example, for expression in insect cells a suitable baculovirus expression vector, such as PFASTBAC, is used to produce recombinant baculovirus particles. The baculovirus particles are amplified and used to infect insect cells to express recombinant protein. For expression in mammalian cells, a vector that will drive expression of the construct in the desired mammalian host cell (e.g., Chinese hamster ovary cells) is used.

The proteins can be purified using any suitable methods. For example, methods for purifying a protein by immunoaffinity chromatography are known in the art. Suitable methods for purifying desired proteins including precipitation and various types of chromatography, such as hydrophobic interaction, ion exchange, affinity, chelating and size exclusion are known in the art. Suitable purification schemes can be created using two or more of these or other suitable methods. If desired, the protein may include a “tag” that facilitates purification, such as an epitope tag or a histidine tag. Such tagged proteins can be purified, for example from conditioned media, by chelating chromatography or affinity chromatography.

### IV. NUCLEIC ACIDS ENCODING PROTEINS

Also provided are nucleic acid molecules that encode a protein described herein. These nucleic acid molecules include DNA, cDNA, and RNA sequences. Nucleic acid molecules that encode only the ectodomain of the protein are also contemplated. The nucleic acid molecule can be incorporated into a vector, such as an expression vector.

The nucleic acid may be a self-replicating RNA molecule. The nucleic acid may include a modified RNA molecule. Also provided are compositions comprising a nucleic acid described herein.

### V. FORMULATIONS AND METHODS OF USE THEREOF

Provided herein are compositions and methods of using the proteins described herein, or a nucleic acid encoding the proteins described herein. For example, the proteins described herein can be delivered directly as a component of an immunogenic composition or a vaccine. Alternatively, nucleic acids that encode the proteins described herein can be administered to produce the protein or immunogenic fragment in vivo. Protein formulations, recombinant nucleic acids (e.g., DNA, RNA, mRNA, self-replicating RNA, or any variation thereof) and/or viral vectors (e.g., live, single-round, non-replicative assembled virions, or otherwise virus-like particles, or alphavirus VRP) that contain sequences encoding the proteins provided herein may be included in a composition.

Immunogenic compositions comprising the proteins described herein are provided. The immunogenic composition can include additional CMV proteins, such as gO, gH, gL, pUL128, pUL130, pUL131, pp65, IE-1, IE-2, pp50, pp150, DNAse, an immunogenic fragment thereof, or a combination thereof. For example, the engineered HCMV gB protein can be combined with CMV pentameric complex comprising: gH or a pentamer-forming fragment thereof, gL or a pentamer-forming fragment thereof, pUL128 or a pentamer-forming fragment thereof, pUL130 or a pentamer-forming fragment thereof, and pUL131 or a pentamer-forming fragment thereof. The engineered HCMV gB protein can also be combined with CMV trimeric complex comprising: gH or a trimer-forming fragment thereof, gL or a trimer-forming fragment thereof, and gO or a trimer-forming fragment thereof.

Also provided are compositions including a polynucleotide that may elicit an immune response in a mammal. The polynucleotide encodes at least one polypeptide of interest, e.g., an antigen. Antigens disclosed herein may be wild type (i.e., derived from the infectious agent) or preferably modified (e.g., engineered, designed or artificial). The nucleic acid molecules described herein, specifically polynucleotides, may encode one or more engineered HCMV gB protein. Such peptides or polypeptides may serve as an antigen or antigenic molecule. The term “nucleic acid” includes any compound that includes a polymer of nucleotides. These polymers are referred to as “polynucleotides.” Exemplary nucleic acids or polynucleotides include, but are not limited to, ribonucleic acids (RNAs), including mRNA, and deoxyribonucleic acids (DNAs).

The composition may include DNA encoding an engineered HCMV gB protein or fragment thereof described herein. The composition may include RNA encoding an engineered HCMV gB protein or fragment thereof described herein. The composition may include an mRNA polynucleotide encoding an engineered HCMV gB protein or fragment thereof described herein. Such compositions may produce the appropriate protein conformation upon translation.

The composition may include at least one polynucleotide encoding two or more antigenic polypeptides or an immunogenic fragment or epitope thereof. The composition may include two or more polynucleotides encoding two or more antigenic polypeptides or immunogenic fragments or epitopes thereof. The one or more antigenic polypeptides may be encoded on a single polynucleotide or may be encoded individually on multiple (e.g., two or more) polynucleotides.

A composition may include (a) a polynucleotide encoding an engineered HCMV gB protein; and (b) a polynucleotide encoding an additional polypeptide.

A composition may include (a) a polynucleotide encoding an engineered HCMV gB protein; and (b) a polynucleotide encoding an additional polypeptide, preferably an HCMV antigenic polypeptide. The additional polypeptide may be selected from HCMV gH, gL, gB, gO, gN, and gM and an immunogenic fragment or epitope thereof. The additional polypeptide may be HCMV pp65. The additional polypeptide may be selected from gH, gL, gO, gM, gN, UL128, UL130, and UL131A, and fragments thereof. The additional polypeptide may be an HCMV gH polypeptide. The additional polypeptide may be an HCMV gL polypeptide. The additional polypeptide may be an HCMV gB polypeptide. The additional polypeptide may be an HCMV gO polypeptide. The additional polypeptide may be an HCMV gN polypeptide. The additional polypeptide may be an HCMV gM polypeptide. The additional polypeptide may be a variant gH polypeptide, a variant gL polypeptide, or a variant gB polypeptide. The variant HCMV gH, gL, or gB polypeptide may be a truncated polypeptide lacking one or more of the following domain sequences: (1) the hydrophobic membrane proximal domain, (2) the transmembrane domain, and (3) the cytoplasmic domain. The truncated HCMV gH, gL, or gB polypeptide may lack the hydrophobic membrane proximal domain, the transmembrane domain, and the cytoplasmic domain. The truncated HCMV gH, gL, or gB polypeptide may include only the ectodomain sequence. An antigenic polypeptide may be an HCMV protein selected from UL83, UL123, UL128, UL130, and UL131A or an immunogenic fragment or epitope thereof. The antigenic polypeptide may be an HCMV UL83 polypeptide. The antigenic polypeptide may be an HCMV UL123 polypeptide. The antigenic polypeptide may be an HCMV UL128 polypeptide. The antigenic polypeptide may be an HCMV UL130 polypeptide. The antigenic polypeptide may be an HCMV UL131A polypeptide.

A composition may include (a) a polynucleotide encoding an engineered HCMV gB protein; and (b) a polynucleotide encoding an additional polypeptide having any one of the amino acid sequences set forth in SEQ ID NOs: 63-74. A composition may include (a) a polynucleotide encoding an engineered HCMV gB protein; and (b) an additional polypeptide or a nucleic acid encoding a polypeptide having at least 90%, at least 91%, at least 92%, at least 93%, at least 95%, at least 96%, at least 97%, at least 98%, or at least 99% sequence identity to any one of the amino acid sequences selected from SEQ ID NOs: 63-74.

The antigenic polypeptide may include two or more HCMV proteins, fragments, or epitopes thereof. The antigenic polypeptide may include two or more glycoproteins, fragments, or epitopes thereof. The antigenic polypeptide may include at least one HCMV polypeptide, fragment or epitope thereof and at least one other HCMV protein, fragment or epitope thereof. The two or more HCMV polypeptides may be encoded by a single RNA polynucleotide. The two or more HCMV polypeptides may be encoded by two or more RNA polynucleotides, for example, each HCMV polypeptide may be encoded by a separate RNA polynucleotide. The two or more HCMV polypeptides may be any combination of HCMV gH, gL, gB, gO, gN, and gM polypeptides or immunogenic fragments or epitopes thereof. The two or more glycoproteins may include pp65 or immunogenic fragments or epitopes thereof; and any combination of HCMV gH, gL, gB, gO, gN, and gM polypeptides or immunogenic fragments or epitopes thereof. The two or more glycoproteins may be any combination of HCMV gB and one or more HCMV polypeptides selected from gH, gL, gO, gN, and gM polypeptides or immunogenic fragments or epitopes thereof. The two or more glycoproteins may be any combination of HCMV gH and one or more HCMV polypeptides selected from gL, gO, gN, and gM polypeptides or immunogenic fragments or epitopes thereof. The two or more glycoproteins can be any combination of HCMV gL and one or more HCMV polypeptides selected from gB, gH, gO, gN, and gM polypeptides or immunogenic fragments or epitopes thereof. The two or more HCMV polypeptides may be gB and gH. The two or more HCMV polypeptides may be gB and gL. The two or more HCMV polypeptides may be gH and gL. The two or more HCMV polypeptides may be gB, gL, and gH. The two or more HCMV proteins may be any combination of HCMV UL83, UL123, UL128, UL130, and UL131A polypeptides or immunogenic fragments or epitopes thereof. The two or more HCMV polypeptides may be UL123 and UL130. The two or more HCMV polypeptides may be UL123 and UL131A. The two or more HCMV polypeptides may be UL130 and UL131A. The two or more HCMV polypeptides may be UL 128, UL130 and UL131A. The two or more HCMV proteins may be any combination of HCMV gB, gH, gL, gO, gM, gN, UL83, UL123, UL128, UL130, and UL131A polypeptides or immunogenic fragments or epitopes thereof. The two or more glycoproteins may be any combination of HCMV gH and one or more HCMV polypeptides selected from gL, UL128, UL130, and UL131A polypeptides or immunogenic fragments or epitopes thereof. The two or more glycoproteins may be any combination of HCMV gL and one or more HCMV polypeptides selected from gH, UL128, UL130, and UL131A polypeptides or immunogenic fragments or epitopes thereof. The two or more HCMV polypeptides may be gL, gH, UL128, UL130 and UL131A. The HCMV gB may be a variant gB, such as any of the engineered HCMV gB proteins disclosed herein.

For compositions that include two or more RNA polynucleotides encoding two or more HCMV antigenic polypeptides or an immunogenic fragment or epitope thereof (either encoded by a single RNA polynucleotide or encoded by two or more RNA polynucleotides, for example, each protein encoded by a separate RNA polynucleotide), the two or more HCMV proteins may be an engineered HCMV gB protein and an HCMV protein selected from gH, gL, gO, gM, gN, UL128, UL130, and UL131A or immunogenic fragments or epitopes thereof. The engineered HCMV protein may be a truncated protein that lacks the hydrophobic membrane proximal domain; lacks the transmembrane domain; lacks the cytoplasmic domain; lacks two or more of the hydrophobic membrane proximal, transmembrane, and cytoplasmic domains; or includes only the ectodomain. A composition may include multimeric RNA polynucleotides encoding at least one HCMV antigenic polypeptide or an immunogenic fragment or epitope thereof. A composition may include at least one RNA polynucleotide encoding at least one HCMV antigenic polypeptide or an immunogenic fragment or epitope thereof, wherein the 5′UTR of the RNA polynucleotide includes a patterned UTR. The patterned UTR may have a repeating or alternating pattern, such as ABABAB or AABBAABBAABB or ABCABCABC or ABCDABCDABCD or variants thereof repeated once, twice, or more than 3 times. In these patterns, each letter, A, B, or C represent a different UTR at the nucleotide level. The 5′ UTR of the RNA polynucleotide (e.g., a first nucleic acid) may have regions of complementarity with a UTR of another RNA polynucleotide (a second nucleic acid). For example, UTR nucleotide sequences of two polynucleotides sought to be joined (e.g., in a multimeric molecule) can be modified to include a region of complementarity such that the two UTRs hybridize to form a multimeric molecule. The 5′ UTR of an RNA polynucleotide encoding an HCMV antigenic polypeptide may be modified to allow the fonnation of a multimeric sequence. The 5′ UTR of an RNA polynucleotide encoding an HCMV protein selected from UL128, UL130, UL131A may be modified to allow the formation of a multimeric sequence. The 5′ UTR of an RNA polynucleotide encoding an HCMV polypeptide may be modified to allow the formation of a multimeric sequence. The 5′ UTR of an RNA polynucleotide encoding an HCMV polypeptide selected from gH, gL, gB, gO, gM, and gN may be modified to allow the formation of a multimeric sequence. A multimer may be a dimer, a trimer, a tetramer, a pentamer, a hexamer, a heptamer, an octamer, a nonamer, a decamer, an 11-mer, a 12-mer, a 13-mer, a 14-mer, a 15-mer, or more.

A composition may include at least one RNA polynucleotide having a single open reading frame encoding two or more (for example, two, three, four, five, or more) HCMV antigenic polypeptides or an immunogenic fragment or epitope thereof. A composition may include at least one RNA polynucleotide having more than one open reading frame, for example, two, three, four, five or more open reading frames encoding two, three, four, five or more HCMV antigenic polypeptides. The at least one RNA polynucleotide may encode two or more HCMV antigenic polypeptides selected from gH, gB, gL, gO, gM, gN, UL83, UL123, UL128, UL130, UL131A, and fragments or epitopes thereof. An RNA polynucleotide that has a single open reading frame encoding two or more (for example, two, three, four, five, or more) HCMV antigenic polypeptides may further comprise additional sequences, for example, a linker sequence or a sequence that aids in the processing of the HCMV RNA transcripts or polypeptides, for example a cleavage site sequence. The additional sequence may be a protease sequence, such as a furin sequence. The additional sequence may be self-cleaving 2A peptide, such as a P2A, E2A, F2A, and T2A sequence. The linker sequences and cleavage site sequences may be interspersed between the sequences encoding HCMV polypeptides.

An RNA polynucleotide includes any nucleic acid sequence selected from any one of the nucleic acid sequences disclosed herein, or homologs thereof having at least 80% (e.g., 85%, 90%, 95%, 98%, 99%) identity with a nucleic acid sequence disclosed herein. An open reading frame may be codon-optimized. A composition may include at least one RNA polynucleotide encoding at least one HCMV antigenic polypeptide or an immunogenic fragment thereof and at least one 5′ terminal cap. A 5′ terminal cap may be 7mG(5′)ppp(5′)NImpNp.

The at least one polynucleotide may have at least one chemical modification. The at least one polynucleotide may further include a second chemical modification. The polynucleotide may be RNA. The at least one polynucleotide having at least one chemical modification may have a 5′ terminal cap. The at least one chemical modification may be selected from pseudouridine, N1-methylpseudouridine, N1-ethylpseudouridine, N1-ethylpseudouridine, 2-thiouridine, 4′-thiouridine, 5-methylcytosine, 2-thio-1-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methoxyuridine, and 2′-O-methyl uridine. At least 80% (e.g., 85%, 90%, 95%, 98%, 99%, 100%) of the uracils in the open reading frame may have a chemical modification, optionally wherein the composition is formulated in a lipid nanoparticle. All of the uracils in the open reading frame may have a chemical modification. The chemical modification may be in the 5-position of the uracil. The chemical modification may be an N1-methyl pseudouridine.

The additional polypeptides or immunogenic fragments encoded by the polynucleotide (e.g., in an mRNA composition) may be selected from gB, gH, gL, gO, gM, gN, UL83, UL123, UL128, UL130, UL131A, pp65, IE1, pp150, pp50, and DNAse antigens.

A first composition and a second composition may be administered to the mammal. A first composition may include a polynucleotide encoding an engineered HCMV gB protein; and a second composition may include a polynucleotide encoding HCMV pp65 or an antigenic fragment or epitope thereof. A first composition may include a polynucleotide encoding an engineered HCMV gB protein; and a second composition may include a polynucleotide encoding at least one polynucleotide encoding an additional polypeptide selected from HCMV gH, gL, UL128, UL130, and UL131A, or antigenic fragments or epitopes thereof.

Provided herein are methods of inducing an immune response in a mammal, the methods including administering to the mammal a composition in an amount effective to induce an immune response, wherein the composition includes a polynucleotide encoding an engineered HCMV gB protein. The immune response may include a T cell response or a B cell response. The method may involve a single administration of the composition. The method may further include administering to the subject a booster dose of the composition. The composition may include a polynucleotide disclosed herein formulated in an effective amount to produce an antigen specific immune response in a mammal.

The immunogenic composition may include an adjuvant. Exemplary adjuvants to enhance effectiveness of the composition include: (1) aluminum salts (alum), such as aluminum hydroxide, aluminum phosphate, aluminum sulfate, etc.; (2) oil-in-water emulsion formulations (with or without other specific adjuvants such as muramyl peptides (see below) or bacterial cell wall components), such as for example (a) MF59 (PCT Publ. No. WO 90/14837), containing 5% Squalene, 0.5% TWEEN 80, and 0.5% Span 85 formulated into submicron particles using a microfluidizer, (b) SAF, containing 10% Squalane, 0.4% Tween 80, 5% pluronic-blocked polymer L121, and thr-MDP either microfluidized into a submicron emulsion or vortexed to generate a larger particle size emulsion, and (c) RIBI™ adjuvant system (RAS), (Ribi Immunochem, Hamilton, Mont.) containing 2% Squalene, 0.2% Tween 80, and one or more bacterial cell wall components from the group consisting of monophosphorylipid A (MPL), trehalose dimycolate (TDM), and cell wall skeleton (CWS), preferably MPL+CWS (DETOX™); (3) saponin adjuvants, such as QS-21, STIMULON™ (Cambridge Bioscience, Worcester, Mass.), which may be used or particles generated therefrom such as ISCOMs (immunostimulating complexes); (4) Complete Freunds Adjuvant (CFA) and Incomplete Freunds Adjuvant (IFA); (5) cytokines, such as interleukins (IL-1, IL-2, etc.), macrophage colony stimulating factor (M-CSF), tumor necrosis factor (TNF), etc.; (6) toll-like receptor agonists; and (7) other substances that act as adjuvants to enhance the effectiveness of the composition. The composition may not include an adjuvant. The composition may further include a lipid nanoparticle. The composition may be formulated in a nanoparticle. The composition may further include a cationic or polycationic compound, including protamine or other cationic peptides or proteins, such as poly-L-lysine (PLL).

Toll-like receptors (TLRs) are expressed in and on dendritic cells and other innate immune cells and are among the most important receptors for stimulating a response to the presence of invading pathogens. Humans have multiple types of TLRs that are similar in structure but recognize different parts of viruses or bacteria. By activating specific TLRs, it is possible to stimulate and control specific types of innate immune responses that can be harnessed to enhance adaptive responses.

TLR9 (CD289) recognizes unmethylated cytidine-phospho-guanosine (CpG) motifs found in microbial DNA, which can be mimicked using synthetic CpG-containing oligodeoxynucleotides (CpG-ODNs). CpG-ODNs are known to enhance antibody production and to stimulate T helper 1 (Thl) cell responses (Coffman et al., Immunity, 33:492-503, 2010). Based on structure and biological function, CpG-ODNs have been divided into three general classes: CpG-A, CpG-B, and CpG-C(Campbell, Methods Mol Biol, 1494:15-27, 2017). The degree of B cell activation varies between the classes with CpG-A ODNs being weak, CpG-C ODNs being good, and CpG-B ODNs being strong B cell activators. Oligonucleotide TLR9 agonists that may be included in the immunogenic compositions provided herein are preferably good B cell activators (CpG-C ODN) or more preferably strong (CpG-B ODN) B cell activators.

Oligonucleotide TLR9 agonists often contain a palindromic sequence following the general formula of: 5′-purine-purine-CG-pyrimidine-pyrimidine-3′ or 5′-purine-purine-CG-pyrimidine-pyrimidine-CG-3′ (U.S. Pat. No. 6,589,940). TLR9 agonism is also observed with certain non-palindromic CpG-enriched phosphorothioate oligonucleotides, but may be affected by changes in the nucleotide sequence. Additionally, TLR9 agonism is abolished by methylation of the cytosine within the CpG dinucleotide. Accordingly in some embodiments, the TLR9 agonist is an oligonucleotide of from 8 to 35 nucleotides in length comprising the sequence 5′-AACGTTCG-3′. In some embodiments, the oligonucleotide is greater than 8, 9, 10, 11, 12, 13, 14, 15, 16, 17, 18, 19, or 20 nucleotides in length, and the oligonucleotide is less than 35, 34, 33, 32, 31, 30, 29, 28, 27, 26, 25, or 24 nucleotides in length. In some embodiments, the TLR9 agonist is an oligonucleotide of from 10 to 35 nucleotides in length comprising the sequence 5′-AACGTTCGAG-3′ (SEQ ID NO: 91). In some embodiments, the oligonucleotide is greater than 10, 11, 12, 13, 14, 15, 16, 17, 18, 19, or 20 nucleotides in length, and the oligonucleotide is less than 35, 34, 33, 32, 31, 30, 29, 28, 27, 26, 25, or 24 nucleotides in length.

For example, the 22-mer phosphorothioate linked oligodeoxynucleotide, CpG 1018, contains specific sequences that can substantially enhance the immune response to co-administered antigens across species (Campbell, Methods Mol Biol, 1494:15-27, 2017; WO 2021/183540, which is incorporated herein by reference in its entirety). CpG 1018 (5′-TGACTGTGAACGTTCGAGATGA-3′, set forth as SEQ ID NO: 92) is a CpG-B ODN that is active in mice, rabbits, dogs, baboons, cynomolgus monkeys, and humans. Thus, in some preferred embodiments, the TLR9 agonist is an oligonucleotide comprising the sequence of SEQ ID NO: 92.

Although the exemplary oligonucleotide TLR9 agonist, CpG 1018, is a CpG-ODN, the present disclosure is not restricted to fully DNA molecules. That is, in some embodiments, the TLR9 agonist is a DNA/RNA chimeric molecule in which the CpG(s) and the palindromic sequence are deoxyribonucleic acids and one or more nucleic acids outside of these regions are ribonucleic acids. In some embodiments, the CpG oligonucleotide is linear. In other embodiments, the CpG oligonucleotide is circular or includes hairpin loop(s). The CpG oligonucleotide may be single stranded or double stranded.

In some embodiments, the CpG oligonucleotide may contain modifications. Modifications include but are not limited to, modifications of the 3′OH or 5′OH group, modifications of the nucleotide base, modifications of the sugar component, and modifications of the phosphate group. Modified bases may be included in the palindromic sequence of the CpG oligonucleotide as long as the modified base(s) maintains the same specificity for its natural complement through Watson-Crick base pairing (e.g., the palindromic portion is still self complementary). In some embodiments, the CpG oligonucleotide comprises a non-canonical base. In some embodiments, the CpG oligonucleotide comprises a modified nucleoside. In some embodiments, the modified nucleoside is selected from the group consisting of 2′-deoxy-7-deazaguanosine, 2′-deoxy-6-thioguanosine, arabinoguanosine, 2′-deoxy-2′substituted-arabinoguanosine, and 2′-0-substituted-arabinoguanosine. In some embodiments, the TLR9 agonist is an oligonucleotide comprising the sequence 5′-TCGiAACGiTTCGi-3′ (SEQ ID NO: 93), in which Gi is 2′-deoxy-7-deazaguanosine. In some embodiments, the oligonucleotide comprises the sequence 5′-TCG_{I}AACG_{I}TTCG_{I}-X-G_{I}CTTG_{I}CAAG₁CT-5′, and in which Gi is 2′-deoxy-7-deazaguanosine and X is glycerol (5′-SEQ ID NO: 93-3′-X-3′-SEQ ID NO: 93-5′).

The CpG oligonucleotide may contain a modification of the phosphate group. For example, in addition to phosphodiester linkages, phosphate modifications include, but are not limited to, methyl phosphonate, phosphorothioate, phosphoramidate (bridging or non-bridging), phosphotriester and phosphorodithioate and may be used in any combination. Other non-phosphate linkages may also be used. In some embodiments, the oligonucleotides comprise only phosphorothioate backbones. In some embodiments, the oligonucleotides comprise only phosphodiester backbones. In some embodiments, the oligonucleotide comprises a combination of phosphate linkages in the phosphate backbone such as a combination of phosphodiester and phosphorothioate linkages. Oligonucleotides with phosphorothioate backbones can be more immunogenic than those with phosphodiester backbones and appear to be more resistant to degradation after injection into the host (Braun et al., J Immunol, 141:2084-2089, 1988; and Latimer et al., Mol Immunol, 32:1057-1064, 1995). The CpG oligonucleotides of the present disclosure include at least one, two or three internucleotide phosphorothioate ester linkages. In some embodiments, when a plurality of CpG oligonucleotide molecules are present in a pharmaceutical composition comprising at least one excipient, both stereoisomers of the phosphorothioate ester linkage are present in the plurality of CpG oligonucleotide molecules. In some embodiments, all of the internucleotide linkages of the CpG oligonucleotide are phosphorothioate linkages, or said another way, the CpG oligonucleotide has a phosphorothioate backbone.

A unit dose of the immunogenic composition, which is typically a 0.5 ml dose, may comprises from about 375 pg to about 6000 pg of the CpG oligonucleotide, preferably from about 750 pg to about 3000 pg of the CpG oligonucleotide. In some embodiments, a 0.5 ml dose of the immunogenic composition comprises greater than about 250, 500, 750, 1000, or 1250 pg of the CpG oligonucleotide, and less than about 6000, 5000, 4000, 3000, or 2000 pg of the CpG oligonucleotide. In some embodiments, a 0.5 ml dose of the immunogenic composition comprises about 375, 750, 1500, 3000 or 6000 pg of the CpG oligonucleotide. In some embodiments, a 0.5 ml dose of the immunogenic composition comprises about 750 pg of the CpG oligonucleotide. In some embodiments, a 0.5 ml dose of the immunogenic composition comprises about 1500 pg of the CpG oligonucleotide. In some embodiments, a 0.5 ml dose of the immunogenic composition comprises about 3000 pg of the CpG oligonucleotide.

The CpG oligonucleotides described herein are in their pharmaceutically acceptable salt form unless otherwise indicated. Exemplary basic salts include ammonium salts, alkali metal salts such as sodium, lithium, and potassium salts, alkaline earth metal salts such as calcium and magnesium salts, zinc salts, salts with organic bases (for example, organic amines) such as N-Me-D-glucamine, N-[1-(2,3-dioleoyloxy)propyl]-N,N,N-trimethylammonium chloride, choline, tromethamine, dicyclohexylamines, t-butyl amines, and salts with amino acids such as arginine, lysine and the like. In some embodiments, the CpG oligonucleotides are in the ammonium, sodium, lithium, or potassium salt form. In one preferred embodiment, the CpG oligonucleotides are in the sodium salt form.

Each of the immunogenic compositions discussed herein may be used alone or in combination with one or more other antigens, the latter either from the same viral pathogen or from another pathogenic source or sources. These compositions may be used for prophylactic (to prevent infection) or therapeutic (to treat disease after infection) purposes.

The composition may include a “pharmaceutically acceptable carrier,” which includes any carrier that does not itself induce the production of antibodies harmful to the individual receiving the composition. Suitable carriers are typically large, slowly metabolized macromolecules such as proteins, polysaccharides, polylactic acids, polyglycolic acids, polymeric amino acids, amino acid copolymers, lipid aggregates (such as oil droplets or liposomes), and inactive virus particles. Such carriers are well known to those of ordinary skill in the art. Additionally, these carriers may function as adjuvants. Furthermore, the antigen may be conjugated to a bacterial toxoid, such as a toxoid from diphtheria, tetanus, cholera, H. pylori, and etc. pathogens.

The composition may include a diluent, such as water, saline, glycerol, ethanol, etc. Additionally, auxiliary substances, such as wetting or emulsifying agents, pH buffering substances, and the like, may be present in such vehicles.

The compositions described herein may include an immunologically effective amount of the polypeptide or polynucleotide, as well as any other of the above-mentioned components, as needed. By “immunologically effective amount,” it is meant that the administration of that amount to an individual, either in a single dose or as part of a series, is effective for eliciting an immune response. The immune response elicited may be sufficient, for example, for treatment and/or prevention and/or reduction in incidence of illness, infection or disease. This amount varies depending upon the health and physical condition of the individual to be treated, the taxonomic group of individual to be treated (e.g., nonhuman primate, primate, etc.), the capacity of the individual's immune system to synthesize antibodies, the degree of protection desired, the formulation of the vaccine, the treating doctor's assessment of the medical situation, and other relevant factors. It is expected that the amount will fall in a relatively broad range that can be determined through routine trials.

The composition may be administered parenterally, e.g., by injection, either subcutaneously or intramuscularly. The composition may be administered to the mammal by intradermal or intramuscular injection. Additional formulations suitable for other modes of administration include oral and pulmonary formulations, nasal formulations, suppositories, and transdermal applications. Oral formulations may be preferred for certain viral proteins. Dosage treatment may be a single dose schedule or a multiple dose schedule. The immunogenic composition may be administered in conjunction with other immunoregulatory agents.

Also provided are methods of eliciting an immune response against cytomegalovirus, comprising administering to a subject in need thereof an immunologically effective amount of the engineered HCMV gB protein and/or an immunogenic composition described herein, which comprises the proteins, DNA molecules, RNA molecules (e.g., self-replicating RNA molecules or mRNA molecules), or VRPs as described above. The immune response may comprise the production of neutralizing antibodies against CMV.

An immune response can comprise a humoral immune response, a cell-mediated immune response, or both. An immune response may be induced against each delivered CMV protein. A cell-mediated immune response can comprise a Helper T-cell (Th) response, a CD8+ cytotoxic T-cell (CTL) response, or both. The immune response may comprise a humoral immune response comprising antibody-presenting B cells, and the antibodies may be neutralizing antibodies.

Neutralizing antibodies block viral infection of cells. CMV infects epithelial cells and also fibroblast cells. The immune response may reduce or prevent infection of both cell types. Neutralizing antibody responses can be complement-dependent or complement-independent. The neutralizing antibody response may be complement-independent. The neutralizing antibody response may be cross-neutralizing; i.e., an antibody generated against an administered composition neutralizes a related herpesvirus or a CMV virus of a strain other than the strain used in the composition.

The polypeptide and/or immunogenic composition described herein may also elicit an effective immune response to reduce the likelihood of a CMV infection of a non-infected mammal, or to reduce symptoms in an infected mammal, e.g., reduce the number of outbreaks, CMV shedding, and risk of spreading the virus to other mammals.

Provided herein are methods for reducing CMV viral shedding in a mammal. The methods may reduce CMV viral shedding in urine in a mammal. The methods may reduce CMV viral shedding in saliva in a mammal. Also provided are methods for reducing CMV viral titers in a mammal. The methods may reduce CMV nucleic acids in serum in a mammal. The term “viral shedding” is used herein according to its plain ordinary meaning in medicine and virology and refers to the production and release of virus from an infected cell. The virus may be released from a cell of a mammal. Virus may be released into the environment from an infected mammal. Virus may be released from a cell within a mammal.

The methods may include administering the engineered HCMV gB protein and/or immunogenic composition described herein to a mammal that is infected with or is at risk of a CMV infection. The reduction in CMV viral shedding in a mammal is as compared to the viral shedding in mammals that were not administered the engineered HCMV gB protein. The reduction in CMV viral shedding in a mammal may be as compared to the viral shedding following an administration of a CMV pentamer alone or following an administration of a CMV pentamer in the absence of the polypeptide.

The mammal may be a human. The human may be a child, such as an infant. The human may be female, including an adolescent female, a female of childbearing age, a female who is planning pregnancy, a pregnant female, and females who recently gave birth. The human may be a transplant patient.

The challenge cytomegalovirus strain may be a human CMV strain. The challenge cytomegalovirus strain may be homologous to the CMV strain from which the engineered gB protein is derived.

A useful measure of antibody potency in the art is “50% neutralization titer.” Another useful measure of antibody potency is any one of the following: a “60% neutralization titer”; a “70% neutralization titer”; a “80% neutralization titer”; and a “90% neutralization titer.” To determine, for example, a 50% neutralizing titer, serum from immunized animals is diluted to assess how dilute serum can be yet retain the ability to block entry of 50% of infectious viruses into cells. For example, a titer of 700 means that serum retained the ability to neutralize 50% of infectious virus after being diluted 700-fold. Thus, higher titers indicate more potent neutralizing antibody responses. The titer may be in a range having a lower limit of about 200, about 400, about 600, about 800, about 1000, about 1500, about 2000, about 2500, about 3000, about 3500, about 4000, about 4500, about 5000, about 5500, about 6000, about 6500, or about 7000. The 50%, 60%, 70%, 80%, or 90% neutralization titer range can have an upper limit of about 400, about 600, about 800, about 1000, about 1500, about 2000, about 2500, about 3000, about 3500, about 4000, about 4500, about 5000, about 5500, about 6000, about 6500, about 7000, about 8000, about 9000, about 10000, about 1 1000, about 12000, about 13000, about 14000, about 15000, about 16000, about 17000, about 18000, about 19000, about 20000, about 21000, about 22000, about 23000, about 24000, about 25000, about 26000, about 27000, about 28000, about 29000, or about 30000. For example, the 50% neutralization titer can be about 3000 to about 6500. “About” means plus or minus 10% of the recited value. Neutralization titer can be measured as described in the specific examples, below.

An immune response can be stimulated by administering proteins, DNA molecules, RNA molecules (e.g., mRNA molecules, self-replicating RNA molecules or nucleoside modified RNA molecules), or VRPs to an individual, typically a mammal, including a human. The immune response induced may be a protective immune response, i.e., the response reduces the risk or severity of or clinical consequences of a CMV infection. Stimulating a protective immune response is particularly desirable in some populations particularly at risk from CMV infection and disease. For example, at-risk populations include solid organ transplant (SOT) patients, bone marrow transplant patients, and hematopoietic stem cell transplant (HSCT) patients. VRPs can be administered to a transplant donor pre-transplant, or a transplant recipient pre- and/or post-transplant. Because vertical transmission from mother to child is a common source of infecting infants, administering VRPs to a patient who is pregnant or can become pregnant is particularly useful.

Any suitable route of administration can be used. For example, a composition can be administered intramuscularly, intraperitoneally, subcutaneously, or transdermally. Administration may be intra-mucosal, such as intra-orally, intra-nasally, intra-vaginally, and intra-rectally. Compositions can be administered according to any suitable schedule.

Also provided herein is a method of inhibiting cytomegalovirus entry into a cell, comprising contacting the cell with the immunogenic composition described herein.

A composition may include an engineered HCMV gB protein described herein. A composition may include a nucleic acid molecule or vector encoding such protein. A composition may include a protein described above and a nucleic acid molecule or vector encoding such protein.

In the case that a nucleic acid molecule encoding an engineered HCMV gB protein is used in a pharmaceutical composition, the nucleic acid molecule may comprise or consist of deoxyribonucleotides and/or ribonucleotides, or analogs thereof, covalently linked together. A nucleic acid molecule as described herein generally contains phosphodiester bonds, although in some cases, nucleic acid analogs are included that may have at least one different linkage, e.g., phosphoramidate, phosphorothioate, phosphorodithioate, or O-methylphophoroamidite linkages, and peptide nucleic acid backbones and linkages. Mixtures of naturally occurring polynucleotides and analogs can be made; alternatively, mixtures of different polynucleotide analogs, and mixtures of naturally occurring polynucleotides and analogs may be made. A nucleic acid molecule may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component. The term also includes both double- and single-stranded molecules. Unless otherwise specified or required, the term polynucleotide encompasses both the double-stranded form and each of two complementary single-stranded forms known or predicted to make up the double-stranded form. A nucleic acid molecule is composed of a specific sequence of four nucleotide bases: adenine (A), cytosine (C), guanine (G), thymine (T), and uracil (U) for thymine when the polynucleotide is RNA. Thus, the term “nucleic acid sequence” is the alphabetical representation of a nucleic acid molecule. Unless otherwise indicated, a particular nucleic acid sequence also implicitly encompasses conservatively modified variants thereof (e.g., degenerate codon substitutions) and complementary sequences as well as the sequence explicitly indicated. Specifically, degenerate codon substitutions may be achieved by generating sequences in which the third position of one or more selected (or all) codons is substituted with mixed-base and/or deoxyinosine residues.

The nucleic acids of the present disclosure may comprise one or more modified nucleosides comprising a modified sugar moiety. Such compounds comprising one or more sugar-modified nucleosides may have desirable properties, such as enhanced nuclease stability or increased binding affinity with a target nucleic acid relative to an oligonucleotide comprising only nucleosides comprising naturally occurring sugar moieties. In some embodiments, modified sugar moieties are substituted sugar moieties. In some embodiments, modified sugar moieties are sugar surrogates. Such sugar surrogates may comprise one or more substitutions corresponding to those of substituted sugar moieties.

In some embodiments, modified sugar moieties are substituted sugar moieties comprising one or more non-bridging sugar substituent, including but not limited to substituents at the 2′ and/or 5′ positions. Examples of sugar substituents suitable for the 2′-position, include, but are not limited to: 2′-F, 2′-OCH3 (“OMe” or “O-methyl”), and 2′-O(CH2)2OCH3 (“MOE”). In certain embodiments, sugar substituents at the 2′ position is selected from allyl, amino, azido, thio, O-allyl, O—C1-C10 alkyl, O—C1-C10 substituted alkyl; OCF3, O(CH2)2SCH3, O(CH2)2-O—N(Rm)(Rn), and O—CH2-C(═O)—N(Rm)(Rn), where each Rm and Rn is, independently, H or substituted or unsubstituted C1-C10 alkyl. Examples of sugar substituents at the 5′-position, include, but are not limited to: 5-methyl (R or S); 5′-vinyl, and 5′-methoxy. In some embodiments, substituted sugars comprise more than one non-bridging sugar substituent, for example, T-F-5′-methyl sugar moieties (see, e.g., PCT International Application WO 2008/101157, for additional 5′,2′-bis substituted sugar moieties and nucleosides).

Nucleosides comprising 2-substituted sugar moieties are referred to as 2′-substituted nucleosides. In some embodiments, a 2′-substituted nucleoside comprises a 2′-substituent group selected from halo, allyl, amino, azido, SH, CN, OCN, CF3, OCF3, O, S, or N(Rm)-alkyl; O, S, or N(Rm)-alkenyl; O, S or N(Rm)-alkynyl; O-alkylenyl-O-alkyl, alkynyl, alkaryl, aralkyl, O-alkaryl, O-aralkyl, O(CH2)2SCH3, O(CH2)2-O—N(Rm)(Rn) or O—CH2-C(═O)—N(Rm)(Rn), where each Rm and Rn is, independently, H, an amino protecting group or substituted or unsubstituted C1-C10 alkyl. These 2′-substituent groups can be further substituted with one or more substituent groups independently selected from hydroxyl, amino, alkoxy, carboxy, benzyl, phenyl, nitro (NO2), thiol, thioalkoxy (S-alkyl), halogen, alkyl, aryl, alkenyl and alkynyl.

In some embodiments, a 2′-substituted nucleoside comprises a 2′-substituent group selected from F, NH2, N3, OCF3, O—CH3, O(CH2)3NH2, CH2-CH═CH2, O—CH2-CH═CH2, OCH2CH2OCH3, O(CH2)2SCH3, O—(CH2)2-O—N(Rm)(Rn), O(CH2)20(CH2)2N(CH3)2, and N-substituted acetamide (O—CH2-C(═O)—N(Rm)(Rn) where each Rm and Rn is, independently, H, an amino protecting group or substituted or unsubstituted C1-C10 alkyl.

In some embodiments, a 2′-substituted nucleoside comprises a sugar moiety comprising a 2′-substituent group selected from F, OCF3, O—CH3, OCH2CH2OCH3, O(CH2)2SCH3, O(CH2)2-O—N(CH3)2, —O(CH2)2O(CH2)2N(CH3)2, and O—CH2-C(═O)—N(H)CH3.

In some embodiments, a 2′-substituted nucleoside comprises a sugar moiety comprising a 2′-substituent group selected from F, O—CH3, and OCH2CH2OCH3.

In some embodiments, nucleosides of the present disclosure comprise one or more unmodified nucleobases. In certain embodiments, nucleosides of the present disclosure comprise one or more modified nucleobases.

In some embodiments, modified nucleobases are selected from: universal bases, hydrophobic bases, promiscuous bases, size-expanded bases, and fluorinated bases as defined herein. 5-substituted pyrimidines, 6-azapyrimidines and N-2, N-6 and O-6 substituted purines, including 2-aminopropyladenine, 5-propynyluracil; 5-propynylcytosine; 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl CH3) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine, 3-deazaguanine and 3-deazaadenine, universal bases, hydrophobic bases, promiscuous bases, size-expanded bases, and fluorinated bases as defined herein. Further modified nucleobases include tricyclic pyrimidines such as phenoxazine cytidine([5,4-b][1,4]benzoxazin-2(3H)-one), phenothiazine cytidine (1H-pyrimido[5,4-b][1,4]benzothiazin-2(3H)-one), G-clamps such as a substituted phenoxazine cytidine (e.g., 9-(2-aminoethoxy)-H-pyrimido [5,4-13][1,4]benzoxazin-2(3H)-one), carbazole cytidine (2H-pyrimido[4,5-b]indol-2-one), pyridoindole cytidine (H-pyrido[3′,2′:4,5]pyrrolo[2,3-d]pyrimidin-2-one). Modified nucleobases may also include those in which the purine or pyrimidine base is replaced with other heterocycles, for example 7-deaza-adenine, 7-deazaguanosine, 2-aminopyridine and 2-pyridone. Further nucleobases include those disclosed in U.S. Pat. No. 3,687,808, those disclosed in The Concise Encyclopedia Of Polymer Science And Engineering, Kroschwitz, J. I., Ed., John Wiley & Sons, 1990, 858-859; those disclosed by Englisch et al., 1991; and those disclosed by Sanghvi, Y. S., 1993.

Representative United States Patents that teach the preparation of certain of the above noted modified nucleobases as well as other modified nucleobases include without limitation, U.S. Pat. Nos. 3,687,808; 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121; 5,596,091; 5,614,617; 5,645,985; 5,681,941; 5,750,692; 5,763,588; 5,830,653 and 6,005,096, each of which is herein incorporated by reference in its entirety.

Additional modifications may also be made at other positions on the oligonucleotide, particularly the 3′ position of the sugar on the 3′ terminal nucleotide and the 5′ position of 5′ terminal nucleotide. For example, one additional modification of the ligand conjugated oligonucleotides of the present disclosure involves chemically linking to the oligonucleotide one or more additional non-ligand moieties or conjugates which enhance the activity, cellular distribution, or cellular uptake of the oligonucleotide. Such moieties include but are not limited to lipid moieties such as a cholesterol moiety (Letsinger et al., 1989), cholic acid (Manoharan et al., 1994), a thioether, e.g., hexyl-5-tritylthiol (Manoharan et al., 1992; Manoharan et al., 1993), a thiocholesterol (Oberhauser et al., 1992), an aliphatic chain, e.g., dodecandiol or undecyl residues (Saison-Behmoaras et al., 1991; Kabanov et al., 1990; Svinarchuk et al., 1993), a phospholipid, e.g., di-hexadecyl-rac-glycerol or triethylammonium 1,2-di-O-hexadecyl-rac-glycero-3-H-phosphonate (Manoharan et al., 1995; Shea et al., 1990), a polyamine or a polyethylene glycol chain (Manoharan et al., 1995), or adamantane acetic acid (Manoharan et al., 1995), a palmityl moiety (Mishra et al., 1995), or an octadecylamine or hexylamino-carbonyl-oxycholesterol moiety (Crooke et al., 1996). In some aspects, a nucleic acid molecule encoding an engineered HCMV gB protein is a modified RNA, such as, for example, a modified mRNA. Modified (m)RNA contemplates certain chemical modifications that confer increased stability and low immunogenicity to mRNAs, thereby facilitating expression of therapeutically important proteins. For instance, N1-methyl-pseudouridine (N1mΨ) outperforms several other nucleoside modifications and their combinations in terms of translation capacity. In some embodiments, the (m)RNA molecules used herein may have the uracils replaced with psuedouracils such as 1-methyl-3′-pseudouridylyl bases. In some embodiments, some of the uracils are replaced, but in other embodiments, all of the uracils have been replaced. The (m)RNA may comprise a 5′ cap, a 5′ UTR element, an optionally codon optimized open reading frame, a 3′ UTR element, and a poly(A) sequence and/or a polyadenylation signal.

The nucleic acid molecule, whether native or modified, may be delivered as a naked nucleic acid molecule or in a delivery vehicle, such as a lipid nanoparticle. A lipid nanoparticle may comprise one or more nucleic acids present in a weight ratio to the lipid nanoparticles from about 5:1 to about 1:100. In some embodiments, the weight ratio of nucleic acid to lipid nanoparticles is from about 5:1, 2.5:1, 1:1, 1:5, 1:10, 1:15, 1:20, 1:25, 1:30, 1:35, 1:40, 1:45, 1:50, 1:60, 1:70, 1:80, 1:90, or 1:100, or any value derivable therein.

In some embodiments, the lipid nanoparticles used herein may contain one, two, three, four, five, six, seven, eight, nine, or ten lipids. These lipids may include triglycerides, phospholipids, steroids or sterols, PEGylated lipids, or a group with an ionizable group such as an alkyl amine and one or more hydrophobic groups such as C6 or greater alkyl groups.

In some aspects of the present disclosure, the lipid nanoparticles are mixed with one or more steroid or a steroid derivative. In some embodiments, the steroid or steroid derivative comprises any steroid or steroid derivative. As used herein, in some embodiments, the term “steroid” is a class of compounds with a four ring 17 carbon cyclic structure which can further comprises one or more substitutions including alkyl groups, alkoxy groups, hydroxy groups, oxo groups, acyl groups, or a double bond between two or more carbon atoms.

In some aspects of the present disclosure, the lipid nanoparticles are mixed with one or more PEGylated lipids (or PEG lipids). In some embodiments, the present disclosure comprises using any lipid to which a PEG group has been attached. In some embodiments, the PEG lipid is a diglyceride which also comprises a PEG chain attached to the glycerol group. In other embodiments, the PEG lipid is a compound which contains one or more C6-C24 long chain alkyl or alkenyl group or a C6-C24 fatty acid group attached to a linker group with a PEG chain. Some non-limiting examples of a PEG lipid includes a PEG modified phosphatidylethanolamine and phosphatidic acid, a PEG ceramide conjugated, PEG modified dialkylamines and PEG modified 1,2-diacyloxypropan-3-amines, PEG modified diacylglycerols and dialkylglycerols. In some embodiments, PEG modified diastearoylphosphatidylethanolamine or PEG modified dimyristoyl-sn-glycerol. In some embodiments, the PEG modification is measured by the molecular weight of PEG component of the lipid. In some embodiments, the PEG modification has a molecular weight from about 100 to about 15,000. In some embodiments, the molecular weight is from about 200 to about 500, from about 400 to about 5,000, from about 500 to about 3,000, or from about 1,200 to about 3,000. The molecular weight of the PEG modification is from about 100, 200, 400, 500, 600, 800, 1,000, 1,250, 1,500, 1,750, 2,000, 2,250, 2,500, 2,750, 3,000, 3,500, 4,000, 4,500, 5,000, 6,000, 7,000, 8,000, 9,000, 10,000, 12,500, to about 15,000. Some non-limiting examples of lipids that may be used in the present disclosure are taught by U.S. Pat. No. 5,820,873, WO 2010/141069, or U.S. Pat. No. 8,450,298, which is incorporated herein by reference.

In some aspects of the present disclosure, the lipid nanoparticles are mixed with one or more phospholipids. In some embodiments, any lipid which also comprises a phosphate group. In some embodiments, the phospholipid is a structure which contains one or two long chain C6-C24 alkyl or alkenyl groups, a glycerol or a sphingosine, one or two phosphate groups, and, optionally, a small organic molecule. In some embodiments, the small organic molecule is an amino acid, a sugar, or an amino substituted alkoxy group, such as choline or ethanolamine. In some embodiments, the phospholipid is a phosphatidylcholine. In some embodiments, the phospholipid is distearoylphosphatidylcholine or dioleoylphosphatidylethanolamine. In some embodiments, other zwitterionic lipids are used, where zwitterionic lipid defines lipid and lipid-like molecules with both a positive charge and a negative charge.

In some aspects of the present disclosure, lipid nanoparticle containing compounds containing lipophilic and cationic components, wherein the cationic component is ionizable, are provided. In some embodiments, the cationic ionizable lipids contain one or more groups which is protonated at physiological pH but may deprotonated and has no charge at a pH above 8, 9, 10, 11, or 12. The ionizable cationic group may contain one or more protonatable amines which are able to form a cationic group at physiological pH. The cationic ionizable lipid compound may also further comprise one or more lipid components such as two or more fatty acids with C6-C24 alkyl or alkenyl carbon groups. These lipid groups may be attached through an ester linkage or may be further added through a Michael addition to a sulfur atom. In some embodiments, these compounds may be a dendrimer, a dendron, a polymer, or a combination thereof.

In some aspects of the present disclosure, composition containing compounds containing lipophilic and cationic components, wherein the cationic component is ionizable, are provided. In some embodiments, ionizable cationic lipids refer to lipid and lipid-like molecules with nitrogen atoms that can acquire charge (pKa). These lipids may be known in the literature as cationic lipids. These molecules with amino groups typically have between 2 and 6 hydrophobic chains, often alkyl or alkenyl such as C6-C24 alkyl or alkenyl groups, but may have at least 1 or more that 6 tails.

In some embodiments, the amount of the lipid nanoparticle with the nucleic acid molecule encapsulated in the pharmaceutical composition is from about 0.1% w/w to about 50% w/w, from about 0.25% w/w to about 25% w/w, from about 0.5% w/w to about 20% w/w, from about 1% w/w to about 15% w/w, from about 2% w/w to about 10% w/w, from about 2% w/w to about 5% w/w, or from about 6% w/w to about 10% w/w. In some embodiments, the amount of the lipid nanoparticle with the nucleic acid molecule encapsulated in the pharmaceutical composition is from about 0.1% w/w, 0.25% w/w, 0.5% w/w, 1% w/w, 2.5% w/w, 5% w/w, 7.5% w/w, 10% w/w, 15% w/w, 20% w/w, 25% w/w, 30% w/w, 35% w/w, 40% w/w, 45% w/w, 50% w/w, 55% w/w, 60% w/w, 65% w/w, 70% w/w, 75% w/w, 80% w/w, 85% w/w, 90% w/w, to about 95% w/w, or any range derivable therein.

In some aspects, the present disclosure comprises one or more sugars formulated into pharmaceutical compositions. In some embodiments, the sugars used herein are saccharides. These saccharides may be used to act as a lyoprotectant that protects the pharmaceutical composition from destabilization during the drying process. These water-soluble excipients include carbohydrates or saccharides such as disaccharides such as sucrose, trehalose, or lactose, a trisaccharide such as fructose, glucose, galactose comprising raffinose, polysaccharides such as starches or cellulose, or a sugar alcohol such as xylitol, sorbitol, or mannitol. In some embodiments, these excipients are solid at room temperature. Some non-limiting examples of sugar alcohols include erythritol, threitol, arabitol, xylitol, ribitol, mannitol, sorbitol, galactitol, fucitol, iditol, inositol, volemitol, isomalt, maltitol, lactitol, maltotritol, maltotetraitol, or a polyglycitol.

In some embodiments, the amount of the sugar in the pharmaceutical composition is from about 25% w/w to about 98% w/w, 40% w/w to about 95% w/w, 50% w/w to about 90% w/w, 50% w/w to about 70% w/w, or from about 80% w/w to about 90% w/w. In some embodiments, the amount of the sugar in the pharmaceutical composition is from about 10% w/w, 15% w/w, 20% w/w, 25% w/w, 30% w/w, 35% w/w, 40% w/w, 45% w/w, 50% w/w, 52.5% w/w, 55% w/w, 57.5% w/w, 60% w/w, 62.5% w/w, 65% w/w, 67.5% w/w, 70% w/w, 75% w/w, 80% w/w, 82.5% w/w, 85% w/w, 87.5% w/w, 90% w/w, to about 95% w/w, or any range derivable therein.

In some embodiments, the pharmaceutically acceptable polymer is a copolymer. The pharmaceutically acceptable polymer may further comprise one, two, three, four, five, or six subunits of discrete different types of polymer subunits. These polymer subunits may include polyoxypropylene, polyoxyethylene, or a similar subunit. In particular, the pharmaceutically acceptable polymer may comprise at least one hydrophobic subunit and at least one hydrophilic subunit. In particular, the copolymer may have hydrophilic subunits on each side of a hydrophobic unit. The copolymer may have a hydrophilic subunit that is polyoxyethylene and a hydrophobic subunit that is polyoxypropylene.

In some embodiments, expression cassettes are employed to express an HCMV gB protein, either for subsequent purification and delivery to a cell/subject, or for use directly in a viral-based delivery approach. Provided herein are expression vectors which contain one or more nucleic acids encoding an HCMV gB protein.

Expression requires that appropriate signals be provided in the vectors and include various regulatory elements such as enhancers/promoters from both viral and mammalian sources that drive expression of the engineered HCMV gB protein in cells. Throughout this application, the term “expression cassette” is meant to include any type of genetic construct containing a nucleic acid coding for a gene product in which part or all of the nucleic acid encoding sequence is capable of being transcribed and translated, i.e., is under the control of a promoter. A “promoter” refers to a DNA sequence recognized by the synthetic machinery of the cell, or introduced synthetic machinery, required to initiate the specific transcription of a gene. The phrase “under transcriptional control” means that the promoter is in the correct location and orientation in relation to the nucleic acid to control RNA polymerase initiation and expression of the gene. An “expression vector” is meant to include expression cassettes comprised in a genetic construct that is capable of replication, and thus including one or more of origins of replication, transcription termination signals, poly-A regions, selectable markers, and multipurpose cloning sites.

The term promoter will be used here to refer to a group of transcriptional control modules that are clustered around the initiation site for RNA polymerase II. Much of the thinking about how promoters are organized derives from analyses of several viral promoters, including those for the HSV thymidine kinase (tk) and SV40 early transcription units. These studies, augmented by more recent work, have shown that promoters are composed of discrete functional modules, each consisting of approximately 7-20 bp of DNA, and containing one or more recognition sites for transcriptional activator or repressor proteins.

At least one module in each promoter functions to position the start site for RNA synthesis. The best known example of this is the TATA box, but in some promoters lacking a TATA box, such as the promoter for the mammalian terminal deoxynucleotidyl transferase gene and the promoter for the SV40 late genes, a discrete element overlying the start site itself helps to fix the place of initiation.

Additional promoter elements regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the tk promoter, the spacing between promoter elements can be increased to 50 bp apart before activity begins to decline. Depending on the promoter, it appears that individual elements can function either co-operatively or independently to activate transcription.

In certain embodiments, viral promotes such as the human cytomegalovirus (CMV) immediate early gene promoter, the SV40 early promoter, the Rous sarcoma virus long terminal repeat, rat insulin promoter and glyceraldehyde-3-phosphate dehydrogenase can be used to obtain high-level expression of the coding sequence of interest. The use of other viral or mammalian cellular or bacterial phage promoters which are well-known in the art to achieve expression of a coding sequence of interest is contemplated as well, provided that the levels of expression are sufficient for a given purpose. By employing a promoter with well-known properties, the level and pattern of expression of the protein of interest following transfection or transformation can be optimized. Further, selection of a promoter that is regulated in response to specific physiologic signals can permit inducible expression of the gene product.

Enhancers are genetic elements that increase transcription from a promoter located at a distant position on the same molecule of DNA. Enhancers are organized much like promoters. That is, they are composed of many individual elements, each of which binds to one or more transcriptional proteins. The basic distinction between enhancers and promoters is operational. An enhancer region as a whole must be able to stimulate transcription at a distance; this need not be true of a promoter region or its component elements. On the other hand, a promoter must have one or more elements that direct initiation of RNA synthesis at a particular site and in a particular orientation, whereas enhancers lack these specificities. Promoters and enhancers are often overlapping and contiguous, often seeming to have a very similar modular organization.

Below is a list of promoters/enhancers and inducible promoters/enhancers that could be used in combination with the nucleic acid encoding a gene of interest in an expression construct. Additionally, any promoter/enhancer combination (as per the Eukaryotic Promoter Data Base EPDB) could also be used to drive expression of the gene. Eukaryotic cells can support cytoplasmic transcription from certain bacterial promoters if the appropriate bacterial polymerase is provided, either as part of the delivery complex or as an additional genetic expression construct.

The promoter and/or enhancer may be, for example, immunoglobulin light chain, immunoglobulin heavy chain, T-cell receptor, HLA DQ a and/or DQ β, β-interferon, interleukin-2, interleukin-2 receptor, MHC class II 5, MHC class II HLA-Dra, β-Actin, muscle creatine kinase (MCK), prealbumin (transthyretin), elastase 1, metallothionein (MT1I), collagenase, albumin, α-fetoprotein, t-globin, β-globin, c-fos, c-HA-ras, insulin, neural cell adhesion molecule (NCAM), α₁-antitrypain, H2B (TH2B) histone, mouse and/or type I collagen, glucose-regulated proteins (GRP94 and GRP78), rat growth hormone, human serum amyloid A (SAA), troponin I (TN 1), platelet-derived growth factor (PDGF), SV40, polyoma, retroviruses, papilloma virus, hepatitis B virus, human immunodeficiency virus, cytomegalovirus (CMV), and gibbon ape leukemia virus.

Where a cDNA insert is employed, one will typically desire to include a polyadenylation signal to effect proper polyadenylation of the gene transcript. Any polyadenylation sequence may be employed such as human growth hormone and SV40 polyadenylation signals. Also contemplated as an element of the expression cassette is a terminator. These elements can serve to enhance message levels and to minimize read through from the cassette into other sequences.

There are a number of ways in which expression vectors may be introduced into cells. In certain embodiments, the expression construct comprises a virus or engineered construct derived from a viral genome. The ability of certain viruses to enter cells via receptor-mediated endocytosis, to integrate into host cell genome and express viral genes stably and efficiently have made them attractive candidates for the transfer of foreign genes into mammalian cells. These have a relatively low capacity for foreign DNA sequences and have a restricted host spectrum. Furthermore, their oncogenic potential and cytopathic effects in permissive cells raise safety concerns. They can accommodate only up to 8 kB of foreign genetic material but can be readily introduced in a variety of cell lines and laboratory animals.

One method for in vivo delivery involves the use of an adenovirus expression vector. “Adenovirus expression vector” is meant to include those constructs containing adenovirus sequences sufficient to (a) support packaging of the construct and (b) to express an engineered HCMV gB protein that has been cloned therein. In this context, expression does not require that the gene product be synthesized.

The expression vector comprises a genetically engineered form of adenovirus. Knowledge of the genetic organization of adenovirus, a 36 kB, linear, double-stranded DNA virus, allows substitution of large pieces of adenoviral DNA with foreign sequences up to 7 kB. In contrast to retrovirus, the adenoviral infection of host cells does not result in chromosomal integration because adenoviral DNA can replicate in an episomal manner without potential genotoxicity. Also, adenoviruses are structurally stable, and no genome rearrangement has been detected after extensive amplification. Adenovirus can infect virtually all epithelial cells regardless of their cell cycle stage. So far, adenoviral infection appears to be linked only to mild disease such as acute respiratory disease in humans.

Adenovirus is particularly suitable for use as a gene transfer vector because of its mid-sized genome, ease of manipulation, high titer, wide target cell range and high infectivity. Both ends of the viral genome contain 100-200 base pair inverted repeats (ITRs), which are cis elements necessary for viral DNA replication and packaging. The early (E) and late (L) regions of the genome contain different transcription units that are divided by the onset of viral DNA replication. The E1 region (ElA and E1B) encodes proteins responsible for the regulation of transcription of the viral genome and a few cellular genes. The expression of the E2 region (E2A and E2B) results in the synthesis of the proteins for viral DNA replication. These proteins are involved in DNA replication, late gene expression and host cell shut-off. The products of the late genes, including the majority of the viral capsid proteins, are expressed only after significant processing of a single primary transcript issued by the major late promoter (MLP). The MLP, (located at 16.8 m.u.) is particularly efficient during the late phase of infection, and all the mRNAs issued from this promoter possess a 5′-tripartite leader (TPL) sequence which makes them preferred mRNAs for translation. In one system, recombinant adenovirus is generated from homologous recombination between shuttle vector and provirus vector. Due to the possible recombination between two proviral vectors, wild-type adenovirus may be generated from this process. Therefore, it is critical to isolate a single clone of virus from an individual plaque and examine its genomic structure.

Generation and propagation of the current adenovirus vectors, which are replication deficient, depend on a unique helper cell line, designated 293, which was transformed from human embryonic kidney cells by Ad5 DNA fragments and constitutively expresses E1 proteins. Since the E3 region is dispensable from the adenovirus genome, the current adenovirus vectors, with the help of 293 cells, carry foreign DNA in either the E1, the D3 or both regions. In nature, adenovirus can package approximately 105% of the wild-type genome, providing capacity for about 2 extra kb of DNA. Combined with the approximately 5.5 kb of DNA that is replaceable in the E1 and E3 regions, the maximum capacity of the current adenovirus vector is under 7.5 kb, or about 15% of the total length of the vector. More than 80% of the adenovirus viral genome remains in the vector backbone and is the source of vector-borne cytotoxicity. Also, the replication deficiency of the E1-deleted virus is incomplete.

Helper cell lines may be derived from human cells such as human embryonic kidney cells, muscle cells, hematopoietic cells or other human embryonic mesenchymal or epithelial cells. Alternatively, the helper cells may be derived from the cells of other mammalian species that are permissive for human adenovirus. Such cells include, e.g., Vero cells or other monkey embryonic mesenchymal or epithelial cells. As stated above, the preferred helper cell line is 293.

The adenoviruses of the disclosure are replication defective, or at least conditionally replication defective. The adenovirus may be of any of the 42 different known serotypes or subgroups A-F. Adenovirus type 5 of subgroup C is one exemplary starting material that may be used to obtain the conditional replication-defective adenovirus vector for use in the present disclosure.

Other viral vectors may be employed as expression constructs in the present disclosure. Vectors derived from viruses such as vaccinia virus, adeno-associated virus (AAV) and herpesviruses may be employed. They offer several attractive features for various mammalian cells.

In embodiments, particular embodiments, the vector is an AAV vector. AAV is a small virus that infects humans and some other primate species. AAV is not currently known to cause disease. The virus causes a very mild immune response, lending further support to its apparent lack of pathogenicity. In many cases, AAV vectors integrate into the host cell genome, which can be important for certain applications, but can also have unwanted consequences. Gene therapy vectors using AAV can infect both dividing and quiescent cells and persist in an extrachromosomal state without integrating into the genome of the host cell, although in the native virus some integration of virally carried genes into the host genome does occur. These features make AAV a very attractive candidate for creating viral vectors for gene therapy, and for the creation of isogenic human disease models. Recent human clinical trials using AAV for gene therapy in the retina have shown promise. AAV belongs to the genus Dependoparvovirus, which in turn belongs to the family Parvoviridae. The virus is a small (20 nm) replication-defective, nonenveloped virus.

Wild-type AAV has attracted considerable interest from gene therapy researchers due to a number of features. Chief amongst these is the virus's apparent lack of pathogenicity. It can also infect non-dividing cells and has the ability to stably integrate into the host cell genome at a specific site (designated AAVS1) in the human chromosome 19. This feature makes it somewhat more predictable than retroviruses, which present the threat of a random insertion and of mutagenesis, which is sometimes followed by development of a cancer. The AAV genome integrates most frequently into the site mentioned, while random incorporations into the genome take place with a negligible frequency. Development of AAVs as gene therapy vectors, however, has eliminated this integrative capacity by removal of the rep and cap from the DNA of the vector. The desired gene together with a promoter to drive transcription of the gene is inserted between the inverted terminal repeats (ITR) that aid in concatemer formation in the nucleus after the single-stranded vector DNA is converted by host cell DNA polymerase complexes into double-stranded DNA. AAV-based gene therapy vectors form episomal concatemers in the host cell nucleus. In non-dividing cells, these concatemers remain intact for the life of the host cell. In dividing cells, AAV DNA is lost through cell division, since the episomal DNA is not replicated along with the host cell DNA. Random integration of AAV DNA into the host genome is detectable but occurs at very low frequency. AAVs also present very low immunogenicity, seemingly restricted to generation of neutralizing antibodies, while they induce no clearly defined cytotoxic response. This feature, along with the ability to infect quiescent cells present their dominance over adenoviruses as vectors for human gene therapy.

The AAV genome is built of single-stranded deoxyribonucleic acid (ssDNA), either positive- or negative-sensed, which is about 4.7 kilobase long. The genome comprises inverted terminal repeats (ITRs) at both ends of the DNA strand, and two open reading frames (ORFs): rep and cap. The former is composed of four overlapping genes encoding Rep proteins required for the AAV life cycle, and the latter contains overlapping nucleotide sequences of capsid proteins: VP1, VP2 and VP3, which interact together to form a capsid of an icosahedral symmetry.

The Inverted Terminal Repeat (ITR) sequences comprise 145 bases each. They were named so because of their symmetry, which was shown to be required for efficient multiplication of the AAV genome. The feature of these sequences that gives them this property is their ability to form a hairpin, which contributes to so-called self-priming that allows primase-independent synthesis of the second DNA strand. The ITRs were also shown to be required for both integration of the AAV DNA into the host cell genome (19th chromosome in humans) and rescue from it, as well as for efficient encapsidation of the AAV DNA combined with generation of a fully assembled, deoxyribonuclease-resistant AAV particles.

With regard to gene therapy, ITRs seem to be the only sequences required in cis next to the therapeutic gene: structural (cap) and packaging (rep) proteins can be delivered in trans. With this assumption many methods were established for efficient production of recombinant AAV (rAAV) vectors containing a reporter or therapeutic gene. However, it was also published that the ITRs are not the only elements required in cis for the effective replication and encapsidation. A few research groups have identified a sequence designated cis-acting Rep-dependent element (CARE) inside the coding sequence of the rep gene. CARE was shown to augment the replication and encapsidation when present in cis.

In some aspects, the present disclosure provides pharmaceutical compositions that contain one or more salts. The salts may be an inorganic potassium or sodium salt such as potassium chloride, sodium chloride, potassium phosphate dibasic, potassium phosphate monobasic, sodium phosphate dibasic, or sodium phosphate monobasic. The pharmaceutical composition may comprise one or more phosphate salts such to generate a phosphate buffer solution. The phosphate buffer solution may be comprise each of the phosphates to buffer a solution to a pH from about 6.8, 6.9, 7.0, 7.1, 7.2, 7.3, 7.4, 7.5, 7.6, 7.7, 7.8, 7.9, or 8.0, or any range derivable therein.

In some aspects, the present disclosure comprises one or more excipients formulated into pharmaceutical compositions. An “excipient” refers to pharmaceutically acceptable carriers that are relatively inert substances used to facilitate administration or delivery of an API into a subject or used to facilitate processing of an API into drug formulations that can be used pharmaceutically for delivery to the site of action in a subject. Furthermore, these compounds may be used as diluents in order to obtain a dosage that can be readily measured or administered to a patient. Non-limiting examples of excipients include polymers, stabilizing agents, surfactants, surface modifiers, solubility enhancers, buffers, encapsulating agents, antioxidants, preservatives, nonionic wetting or clarifying agents, viscosity increasing agents, and absorption-enhancing agents.

The term “pharmaceutically acceptable” may mean approved by a regulatory agency of the Federal or a state government or listed in the U.S. Pharmacopeia or other generally recognized pharmacopeia for use in animals, and more particularly in humans. The term “carrier” refers to a diluent, excipient, or vehicle with which the therapeutic is administered. Such pharmaceutical carriers can be sterile liquids, such as water and can preferably include an adjuvant. Water is a particular carrier when the pharmaceutical composition is administered by injections, such an intramuscular injection. Saline solutions and aqueous dextrose and glycerol solutions can also be employed as liquid carriers, particularly for injectable solutions. Other suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like.

The composition, if desired, can also contain minor amounts of wetting or emulsifying agents, or pH buffering agents. These compositions can take the form of solutions, suspensions, emulsion, tablets, pills, capsules, powders, sustained-release formulations and the like. Oral formulations can include standard carriers such as pharmaceutical grades of mannitol, lactose, starch, magnesium stearate, sodium saccharine, cellulose, magnesium carbonate, etc. Examples of suitable pharmaceutical agents are described in “Remington's Pharmaceutical Sciences.” Such compositions will contain a prophylactically or therapeutically effective amount of the antibody or fragment thereof, preferably in purified form, together with a suitable amount of carrier so as to provide the form for proper administration to the patient. The formulation should suit the mode of administration, which can be oral, intravenous, intraarterial, intrabuccal, intranasal, nebulized, bronchial inhalation, or delivered by mechanical ventilation.

Engineered proteins or nucleic acids encoding engineered proteins of the present disclosure, as described herein, can be formulated for parenteral administration, e.g., formulated for injection via the intradermal, intravenous, intramuscular, subcutaneous, intra-tumoral or even intraperitoneal routes. The formulation could alternatively be administered by a topical route directly to the mucosa, for example by nasal drops, inhalation, or by nebulizer. Pharmaceutically acceptable salts include the acid salts and those which are formed with inorganic acids such as, for example, hydrochloric or phosphoric acids, or such organic acids as acetic, oxalic, tartaric, mandelic, and the like. Salts formed with the free carboxyl groups may also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

Generally, the ingredients of compositions of the disclosure are supplied either separately or mixed together in unit dosage form, for example, as a dry lyophilized powder or water-free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The compositions of the disclosure can be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

Dosage can be by a single dose schedule or a multiple dose schedule. Multiple doses may be used in a primary immunization schedule and/or in a booster immunization schedule. In a multiple dose schedule the various doses may be given by the same or different routes. Multiple doses will typically be administered at least 1 week apart (e.g., about 2 weeks, about 3 weeks, about 4 weeks, about 6 weeks, about 8 weeks, about 10 weeks, about 12 weeks, about 16 weeks, etc.).

The compositions disclosed herein may be used to treat both children and adults. Thus, a human subject may be less than 1 year old, 1-5 years old, 5-16 years old, 16-55 years old, 55-65 years old, or at least 65 years old.

Preferred routes of administration include, but are not limited to, intramuscular, intraperitoneal, intradermal, subcutaneous, intravenous, intraarterial, and intraoccular injection. Particularly preferred routes of administration include intramuscular, intradermal and subcutaneous injection.

### VI. ANTIBODIES AND DIAGNOSTIC USES

The polypeptides described above may be used to produce antibodies, both polyclonal and monoclonal. If polyclonal antibodies are desired, a selected mammal (e.g., mouse, rabbit, goat, guinea pig, horse, etc.) is immunized with an immunogenic polypeptide bearing a CMV epitope(s). Serum from the immunized animal is collected and treated according to known procedures. If serum containing polyclonal antibodies to a CMV epitope contains antibodies to other antigens, the polyclonal antibodies can be purified by immunoaffinity chromatography. Techniques for producing and processing polyclonal antisera are known in the art.

Monoclonal antibodies directed against CMV epitopes can also be readily produced by one skilled in the art. The general methodology for making monoclonal antibodies by hybridomas is known. Immortal antibody-producing cell lines can be created by cell fusion, and also by other techniques such as direct transformation of B lymphocytes with oncogenic DNA, or transfection with Epstein-Barr virus. Panels of monoclonal antibodies produced against CMV epitopes can be screened for various properties; i.e., for isotype, epitope affinity, etc.

Antibodies, both monoclonal and polyclonal, which are directed against CMV epitopes are particularly useful in diagnosis, and those which are neutralizing are useful in passive immunotherapy. Monoclonal antibodies, in particular, may be used to raise anti-idiotype antibodies.

Both the polypeptides which react inmmunologically with serum containing CMV antibodies, and the antibodies raised against these polypeptides, may be useful in immunoassays to detect the presence of CMV antibodies, or the presence of the virus, in biological samples, including for example, blood or serum samples. Design of the immunoassays is subject to a great deal of variation, and a variety of these are known in the art. For example, the immunoassay may utilize the polypeptide having the sequence set forth in any one of SEQ ID NOs: 7-62.

Alternatively, the immunoassay may use a combination of viral antigens derived from the polypeptides described herein. It may use, for example, a monoclonal antibody directed towards at least one polypeptide described herein, a combination of monoclonal antibodies directed towards the polypeptides described herein, monoclonal antibodies directed towards different viral antigens, polyclonal antibodies directed towards the polypeptides described herein, or polyclonal antibodies directed towards different viral antigens. Protocols may be based, for example, upon competition, or direct reaction, or may be sandwich type assays. Protocols may also, for example, use solid supports, or may be by immunoprecipitation. Most assays involve the use of labeled antibody or polypeptide; the labels may be, for example, fluorescent, chemiluminescent, radioactive, or dye molecules. Assays which amplify the signals from the probe are also known; examples of which are assays which utilize biotin and avidin, and enzyme-labeled and mediated immunoassays, such as ELISA assays.

Kits suitable for immunodiagnosis and containing the appropriate labeled reagents are constructed by packaging the appropriate materials, including the engineered HCMV gB proteins containing CMV epitopes or antibodies directed against epitopes in suitable containers, along with the remaining reagents and materials required for the conduct of the assay, as well as a suitable set of assay instructions.

The polynucleotide probes can also be packaged into diagnostic kits. Diagnostic kits include the probe DNA, which may be labeled; alternatively, the probe DNA may be unlabeled and the ingredients for labeling may be included in the kit. The kit may also contain other suitably packaged reagents and materials needed for the particular hybridization protocol, for example, standards, as well as instructions for conducting the test.

### VII. IMMUNODETECTION METHODS

The present disclosure concerns immunodetection methods for binding, purifying, removing, quantifying and otherwise generally detecting HCMV gB protein. While such methods can be applied in a traditional sense, another use will be in quality control and monitoring of vaccine stocks, where antibodies according to the present disclosure can be used to assess the amount or integrity (i.e., long term stability) of antigens. Alternatively, the methods may be used to screen various antibodies for appropriate/desired reactivity profiles.

Some immunodetection methods include enzyme linked immunosorbent assay (ELISA), radioimmunoassay (RIA), immunoradiometric assay, fluoroimmunoassay, chemiluminescent assay, bioluminescent assay, and Western blot to mention a few. In particular, a competitive assay for the detection and quantitation of HCMV gB protein also is provided. The steps of various useful immunodetection methods have been described in the scientific literature, such as, e.g., Doolittle and Ben-Zeev (1999), Gulbis and Galand (1993), De Jager et al. (1993), and Nakamura et al. (1987). In general, the immunobinding methods include obtaining a sample suspected of containing HCMV gB protein, and contacting the sample with a first antibody in accordance with the present disclosure, as the case may be, under conditions effective to allow the formation of immunocomplexes.

These methods include methods for detecting or purifying HCMV gB protein from a sample. The antibody will preferably be linked to a solid support, such as in the form of a column matrix, and the sample suspected of containing the HCMV gB protein will be applied to the immobilized antibody. The unwanted components will be washed from the column, leaving the HCMV gB protein-expressing cells immunocomplexed to the immobilized antibody, which is then collected by removing the organism or antigen from the column.

The immunobinding methods also include methods for detecting and quantifying the amount of HCMV gB protein or related components in a sample and the detection and quantification of any immune complexes formed during the binding process. Here, one would obtain a sample suspected of containing HCMV gB protein and contact the sample with an antibody that binds HCMV gB protein or components thereof, followed by detecting and quantifying the amount of immune complexes formed under the specific conditions. In terms of antigen detection, the biological sample analyzed may be any sample that is suspected of containing HCMV gB protein, such as a tissue section or specimen, a homogenized tissue extract, a biological fluid (e.g., a nasal swab), including blood and serum, or a secretion, such as feces or urine.

Contacting the chosen biological sample with the antibody under effective conditions and for a period of time sufficient to allow the formation of immune complexes (primary immune complexes) is generally a matter of simply adding the antibody composition to the sample and incubating the mixture for a period of time long enough for the antibodies to form immune complexes with, i.e., to bind to HCMV gB protein. After this time, the sample-antibody composition, such as a tissue section, ELISA plate, dot blot or Western blot, will generally be washed to remove any non-specifically bound antibody species, allowing only those antibodies specifically bound within the primary immune complexes to be detected.

In general, the detection of immunocomplex formation is well known in the art and may be achieved through the application of numerous approaches. These methods are generally based upon the detection of a label or marker, such as any of those radioactive, fluorescent, biological and enzymatic tags. Patents concerning the use of such labels include U.S. Pat. Nos. 3,817,837, 3,850,752, 3,939,350, 3,996,345, 4,277,437, 4,275,149 and 4,366,241. Of course, one may find additional advantages through the use of a secondary binding ligand such as a second antibody and/or a biotin/avidin ligand binding arrangement, as is known in the art.

The antibody employed in the detection may itself be linked to a detectable label, wherein one would then simply detect this label, thereby allowing the amount of the primary immune complexes in the composition to be determined. Alternatively, the first antibody that becomes bound within the primary immune complexes may be detected by means of a second binding ligand that has binding affinity for the antibody. In these cases, the second binding ligand may be linked to a detectable label. The second binding ligand is itself often an antibody, which may thus be termed a “secondary” antibody. The primary immune complexes are contacted with the labeled, secondary binding ligand, or antibody, under effective conditions and for a period of time sufficient to allow the formation of secondary immune complexes. The secondary immune complexes are then generally washed to remove any non-specifically bound labeled secondary antibodies or ligands, and the remaining label in the secondary immune complexes is then detected.

Further methods include the detection of primary immune complexes by a two-step approach. A second binding ligand, such as an antibody that has binding affinity for the antibody, is used to form secondary immune complexes, as described above. After washing, the secondary immune complexes are contacted with a third binding ligand or antibody that has binding affinity for the second antibody, again under effective conditions and for a period of time sufficient to allow the formation of immune complexes (tertiary immune complexes). The third ligand or antibody is linked to a detectable label, allowing detection of the tertiary immune complexes thus formed. This system may provide for signal amplification if this is desired.

One method of immunodetection uses two different antibodies. A first biotinylated antibody is used to detect the target antigen, and a second antibody is then used to detect the biotin attached to the complexed biotin. In that method, the sample to be tested is first incubated in a solution containing the first step antibody. If the target antigen is present, some of the antibody binds to the antigen to form a biotinylated antibody/antigen complex. The antibody/antigen complex is then amplified by incubation in successive solutions of streptavidin (or avidin), biotinylated DNA, and/or complementary biotinylated DNA, with each step adding additional biotin sites to the antibody/antigen complex. The amplification steps are repeated until a suitable level of amplification is achieved, at which point the sample is incubated in a solution containing the second step antibody against biotin. This second step antibody is labeled, as for example with an enzyme that can be used to detect the presence of the antibody/antigen complex by histoenzymology using a chromogen substrate. With suitable amplification, a conjugate can be produced which is macroscopically visible.

Another known method of immunodetection takes advantage of the immuno-PCR (Polymerase Chain Reaction) methodology. The PCR method is similar to the Cantor method up to the incubation with biotinylated DNA, however, instead of using multiple rounds of streptavidin and biotinylated DNA incubation, the DNA/biotin/streptavidin/antibody complex is washed out with a low pH or high salt buffer that releases the antibody. The resulting wash solution is then used to carry out a PCR reaction with suitable primers with appropriate controls. At least in theory, the enormous amplification capability and specificity of PCR can be utilized to detect a single antigen molecule.

### A. ELISAs

Inmunoassays, in their most simple and direct sense, are binding assays. Certain preferred immunoassays are the various types of enzyme linked immunosorbent assays (ELISAs) and radioimmunoassays (RIA) known in the art. Immunohistochemical detection using tissue sections is also particularly useful. However, it will be readily appreciated that detection is not limited to such techniques, and western blotting, dot blotting, FACS analyses, and the like may also be used.

In one exemplary ELISA, the antibodies of the disclosure are immobilized onto a selected surface exhibiting protein affinity, such as a well in a polystyrene microtiter plate. Then, a test composition suspected of containing the HCMV gB protein is added to the wells. After binding and washing to remove non-specifically bound immune complexes, the bound antigen may be detected. Detection may be achieved by the addition of another anti-HCMV gB protein antibody that is linked to a detectable label. This type of ELISA is a simple “sandwich ELISA.” Detection may also be achieved by the addition of a second anti-HCMV gB protein antibody, followed by the addition of a third antibody that has binding affinity for the second antibody, with the third antibody being linked to a detectable label.

In another exemplary ELISA, the samples suspected of containing the HCMV gB protein (e.g., potentially infected cells) are immobilized onto the well surface and then contacted with the anti-HCMV gB protein antibodies of the disclosure. After binding and washing to remove non-specifically bound immune complexes, the bound anti-HCMV gB protein antibodies are detected. Where the initial anti-HCMV gB protein antibodies are linked to a detectable label, the immune complexes may be detected directly. Again, the immune complexes may be detected using a second antibody that has binding affinity for the first anti-HCMV gB protein antibody, with the second antibody being linked to a detectable label.

Irrespective of the format employed, ELISAs have certain features in common, such as coating, incubating and binding, washing to remove non-specifically bound species, and detecting the bound immune complexes. These are described below.

In coating a plate with either antigen or antibody, one will generally incubate the wells of the plate with a solution of the antigen or antibody, either overnight or for a specified period of hours. The wells of the plate will then be washed to remove incompletely adsorbed material. Any remaining available surfaces of the wells are then “coated” with a nonspecific protein that is antigenically neutral with regard to the test antisera. These include bovine serum albumin (BSA), casein or solutions of milk powder. The coating allows for blocking of nonspecific adsorption sites on the immobilizing surface and thus reduces the background caused by nonspecific binding of antisera onto the surface.

In ELISAs, it is probably more customary to use a secondary or tertiary detection means rather than a direct procedure. Thus, after binding of a protein or antibody to the well, coating with a non-reactive material to reduce background, and washing to remove unbound material, the immobilizing surface is contacted with the biological sample to be tested under conditions effective to allow immune complex (antigen/antibody) formation. Detection of the immune complex then requires a labeled secondary binding ligand or antibody, and a secondary binding ligand or antibody in conjunction with a labeled tertiary antibody or a third binding ligand.

“Under conditions effective to allow immune complex (antigen/antibody) formation” means that the conditions preferably include diluting the antigens and/or antibodies with solutions such as BSA, bovine gamma globulin (BGG) or phosphate buffered saline (PBS)/Tween. These added agents also tend to assist in the reduction of nonspecific background.

The “suitable” conditions also mean that the incubation is at a temperature or for a period of time sufficient to allow effective binding. Incubation steps are typically from about 1 to 2 to 4 hours or so, at temperatures preferably on the order of 25° C. to 27° C., or may be overnight at about 4° C. or so.

Following all incubation steps in an ELISA, the contacted surface is washed so as to remove non-complexed material. A preferred washing procedure includes washing with a solution such as PBS/Tween, or borate buffer. Following the formation of specific immune complexes between the test sample and the originally bound material, and subsequent washing, the occurrence of even minute amounts of immune complexes may be determined.

To provide a detecting means, the second or third antibody will have an associated label to allow detection. Preferably, this will be an enzyme that will generate color development upon incubating with an appropriate chromogenic substrate. Thus, for example, one will desire to contact or incubate the first and second immune complex with a urease, glucose oxidase, alkaline phosphatase or hydrogen peroxidase-conjugated antibody for a period of time and under conditions that favor the development of further immune complex formation (e.g., incubation for 2 hours at room temperature in a PBS-containing solution such as PBS-Tween).

After incubation with the labeled antibody, and subsequent to washing to remove unbound material, the amount of label is quantified, e.g., by incubation with a chromogenic substrate such as urea, or bromocresol purple, or 2,2′-azino-di-(3-ethyl-benzthiazoline-6-sulfonic acid (ABTS), or H₂O₂, in the case of peroxidase as the enzyme label. Quantification is then achieved by measuring the degree of color generated, e.g., using a visible spectra spectrophotometer.

### B. Western Blot

The Western blot (alternatively, protein immunoblot) is an analytical technique used to detect specific proteins in a given sample of tissue homogenate or extract.

It uses gel electrophoresis to separate native or denatured proteins by the length of the polypeptide (denaturing conditions) or by the 3-D structure of the protein (native/non-denaturing conditions). The proteins are then transferred to a membrane (typically nitrocellulose or PVDF), where they are probed (detected) using antibodies specific to the target protein.

Samples may be taken from whole tissue or from cell culture. In most cases, solid tissues are first broken down mechanically using a blender (for larger sample volumes), using a homogenizer (smaller volumes), or by sonication. Cells may also be broken open by one of the above mechanical methods. Assorted detergents, salts, and buffers may be employed to encourage lysis of cells and to solubilize proteins. Protease and phosphatase inhibitors are often added to prevent the digestion of the sample by its own enzymes. Tissue preparation is often done at cold temperatures to avoid protein denaturing.

The proteins of the sample are separated using gel electrophoresis. Separation of proteins may be by isoelectric point (pI), molecular weight, electric charge, or a combination of these factors. The nature of the separation depends on the treatment of the sample and the nature of the gel. This is a very useful way to determine a protein. It is also possible to use a two-dimensional (2-D) gel which spreads the proteins from a single sample out in two dimensions. Proteins are separated according to isoelectric point (pH at which they have neutral net charge) in the first dimension, and according to their molecular weight in the second dimension.

In order to make the proteins accessible to antibody detection, they are moved from within the gel onto a membrane made of nitrocellulose or polyvinylidene difluoride (PVDF). The membrane is placed on top of the gel, and a stack of filter papers placed on top of that. The entire stack is placed in a buffer solution which moves up the paper by capillary action, bringing the proteins with it. Another method for transferring the proteins is called electroblotting and uses an electric current to pull proteins from the gel into the PVDF or nitrocellulose membrane. The proteins move from within the gel onto the membrane while maintaining the organization they had within the gel. As a result of this blotting process, the proteins are exposed on a thin surface layer for detection (see below). Both varieties of membrane are chosen for their non-specific protein binding properties (i.e., binds all proteins equally well). Protein binding is based upon hydrophobic interactions, as well as charged interactions between the membrane and protein. Nitrocellulose membranes are cheaper than PVDF, but are far more fragile and do not stand up well to repeated probing. The uniformity and overall effectiveness of transfer of protein from the gel to the membrane can be checked by staining the membrane with Coomassie Brilliant Blue or Ponceau S dyes. Once transferred, proteins are detected using labeled primary antibodies, or unlabeled primary antibodies followed by indirect detection using labeled protein A or secondary labeled antibodies binding to the Fc region of the primary antibodies.

### C. Immunohistochemistry

The antibodies of the present disclosure may also be used in conjunction with both fresh-frozen and/or formalin-fixed, paraffin-embedded tissue blocks prepared for study by immunohistochemistry (IHC). The method of preparing tissue blocks from these particulate specimens has been successfully used in previous IHC studies of various prognostic factors, and is well known to those of skill in the art (Brown et al., 1990; Abbondanzo et al., 1990; Allred et al., 1990).

Briefly, frozen-sections may be prepared by rehydrating 50 ng of frozen “pulverized” tissue at room temperature in phosphate buffered saline (PBS) in small plastic capsules; pelleting the particles by centrifugation; resuspending them in a viscous embedding medium (OCT); inverting the capsule and/or pelleting again by centrifugation; snap-freezing in −70° C. isopentane; cutting the plastic capsule and/or removing the frozen cylinder of tissue; securing the tissue cylinder on a cryostat microtome chuck; and/or cutting 25-50 serial sections from the capsule. Alternatively, whole frozen tissue samples may be used for serial section cuttings.

Permanent-sections may be prepared by a similar method involving rehydration of the 50 mg sample in a plastic microfuge tube; pelleting; resuspending in 10% formalin for 4 hours fixation; washing/pelleting; resuspending in warm 2.5% agar; pelleting; cooling in ice water to harden the agar; removing the tissue/agar block from the tube; infiltrating and/or embedding the block in paraffin; and/or cutting up to 50 serial permanent sections. Again, whole tissue samples may be substituted.

### D. Immunodetection Kits

In still further embodiments, the present disclosure concerns immunodetection kits for use with the immunodetection methods described above. As the antibodies may be used to detect HCMV gB protein, the antibodies may be included in the kit. The immunodetection kits will thus comprise, in suitable container means, a first antibody that binds to an HCMV gB protein, and optionally an immunodetection reagent.

In certain embodiments, the antibody may be pre-bound to a solid support, such as a column matrix and/or well of a microtitre plate. The immunodetection reagents of the kit may take any one of a variety of forms, including those detectable labels that are associated with or linked to the given antibody. Detectable labels that are associated with or attached to a secondary binding ligand are also contemplated. Exemplary secondary ligands are those secondary antibodies that have binding affinity for the first antibody.

Further suitable immunodetection reagents for use in the present kits include the two-component reagent that comprises a secondary antibody that has binding affinity for the first antibody, along with a third antibody that has binding affinity for the second antibody, the third antibody being linked to a detectable label. As noted above, a number of exemplary labels are known in the art and all such labels may be employed in connection with the present disclosure.

The kits may further comprise a suitably aliquoted composition of HCMV gB protein, whether labeled or unlabeled, as may be used to prepare a standard curve for a detection assay. The kits may contain antibody-label conjugates either in fully conjugated form, in the form of intermediates, or as separate moieties to be conjugated by the user of the kit. The components of the kits may be packaged either in aqueous media or in lyophilized form.

The container means of the kits will generally include at least one vial, test tube, flask, bottle, syringe or other container means, into which the antibody may be placed, or preferably, suitably aliquoted. The kits of the present disclosure will also typically include a means for containing the antibody, antigen, and any other reagent containers in close confinement for commercial sale. Such containers may include injection or blow-molded plastic containers into which the desired vials are retained.

### E. Flow Cytometry and FACS

The antibodies of the present disclosure may also be used in flow cytometry or FACS. Flow cytometry is a laser- or impedance-based technology employed in many detection assays, including cell counting, cell sorting, biomarker detection and protein engineering. The technology suspends cells in a stream of fluid and passing them through an electronic detection apparatus, which allows simultaneous multiparametric analysis of the physical and chemical characteristics of up to thousands of particles per second. Flow cytometry is routinely used in the diagnosis disorders, especially blood cancers, but has many other applications in basic research, clinical practice and clinical trials.

Fluorescence-activated cell sorting (FACS) is a specialized type of cytometry. It provides a method for sorting a heterogenous mixture of biological cells into two or more containers, one cell at a time, based on the specific light scattering and fluorescent characteristics of each cell. In general, the technology involves a cell suspension entrained in the center of a narrow, rapidly flowing stream of liquid. The flow is arranged so that there is a large separation between cells relative to their diameter. A vibrating mechanism causes the stream of cells to break into individual droplets. Just before the stream breaks into droplets, the flow passes through a fluorescence measuring station where the fluorescence of each cell is measured. An electrical charging ring is placed just at the point where the stream breaks into droplets. A charge is placed on the ring based immediately prior to fluorescence intensity being measured, and the opposite charge is trapped on the droplet as it breaks form the stream. The charged droplets then fall through an electrostatic deflection system that diverts droplets into containers based upon their charge.

In certain embodiments, to be used in flow cytometry or FACS, the antibodies of the present disclosure are labeled with fluorophores and then allowed to bind to the cells of interest, which are analyzed in a flow cytometer or sorted by a FACS machine.

### VIII. DEFINITIONS

It is to be understood that both the foregoing general description and the following detailed description are exemplary and explanatory only and are not restrictive of the invention as claimed. In this application, the use of the singular includes the plural unless specifically stated otherwise. In this application, the use of “or” means “and/or” unless stated otherwise. Furthermore, the use of the term “including”, as well as other forms, such as “includes” and “included”, is not limiting. Also, terms such as “element” or “component” encompass both elements and components comprising one unit and elements and components that comprise more than one subunit unless specifically stated otherwise. Also, the use of the term “portion” can include part of a moiety or the entire moiety.

As used herein, the singular forms “a”, “an” and “the” include plural references unless the context clearly dictates otherwise. As used herein the specification, “a” or “an” may mean one or more. As used herein in the claim(s), when used in conjunction with the word “comprising,” the words “a” or “an” may mean one or more than one.

The term “about” as used herein when referring to a measurable value such as an amount, a temporal duration, and the like, is meant to encompass variations of up to ±10% from the specified value. Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the disclosed subject matter. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contain certain errors necessarily resulting from the standard deviation found in their respective testing measurements.

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is therefore well below 0.05%, preferably below 0.01%. Most preferred is a composition in which no amount of the specified component can be detected with standard analytical methods.

The size of the native form of HCMV gB depends on the size of the open reading frame (ORF) which may vary a little according to the strain. For example, the ORF of AD169 strain, which is 2717 bp long, encodes a full length gB of 906 amino acids, whereas the ORF of Towne and Merlin strains encode a full length gB of 907 amino acids. Although the present disclosure is applicable to gB proteins originating from any HCMV strain, in order to facilitate its understanding, when referring to amino acid positions in the present specification, the numbering is given in relation to the amino acid sequence of the gB protein of SEQ ID NO:1 originating from the clinical isolate Towne strain, unless otherwise stated. The present disclosure is not, however, limited to the HCMV Towne strain.

Therefore, when referring to “comparable” or “corresponding” or “relative” amino acid positions or specific amino acids in a gB protein of any other HCMV strain (also relevant in the context of nucleic acids), such “comparable” or “corresponding” or “relative” residues (or nucleic acids) can be determined by those of ordinary skill in the art using known information (see US 2013/0216613 and US 2018/0265551) and by sequence alignment using readily available and well-known alignment algorithms (such as BLAST, using default settings; ClustalW2, using default settings). Accordingly, when referring to a “HCMV gB protein”, it is to be understood as a HCMV gB protein from any strain. The actual residue location number and residue identity may have to be adjusted for gB proteins from HCMV other than HCMV Towne strain, depending on the actual sequence alignment.

The term “antibody” refers to an intact immunoglobulin of any isotype, or a fragment thereof that can compete with the intact antibody for specific binding to the target antigen, and includes, for instance, chimeric, humanized, fully human, and bispecific antibodies. An “antibody” is a species of an antigen binding protein. An intact antibody will generally comprise at least two full-length heavy chains and two full-length light chains, but in some instances can include fewer chains such as antibodies naturally occurring in camelids which can comprise only heavy chains. Antibodies can be derived solely from a single source, or can be “chimeric,” that is, different portions of the antibody can be derived from two different antibodies as described further below. The antigen binding proteins, antibodies, or binding fragments can be produced in hybridomas, by recombinant DNA techniques, or by enzymatic or chemical cleavage of intact antibodies. Unless otherwise indicated, the term “antibody” includes, in addition to antibodies comprising two full-length heavy chains and two full-length light chains, derivatives, variants, fragments, and muteins thereof, examples of which are described below. Furthermore, unless explicitly excluded, antibodies include monoclonal antibodies, bispecific antibodies, minibodies, domain antibodies, synthetic antibodies (sometimes referred to herein as “antibody mimetics”), chimeric antibodies, humanized antibodies, human antibodies, antibody fusions (sometimes referred to herein as “antibody conjugates”), and fragments thereof, respectively. In some embodiments, the term also encompasses peptibodies.

Naturally occurring antibody structural units typically comprise a tetramer. Each such tetramer typically is composed of two identical pairs of polypeptide chains, each pair having one full-length “light” (in certain embodiments, about 25 kDa) and one full-length “heavy” chain (in certain embodiments, about 50-70 kDa). The amino-terminal portion of each chain typically includes a variable region of about 100 to 110 or more amino acids that typically is responsible for antigen recognition. The carboxy-terminal portion of each chain typically defines a constant region that can be responsible for effector function. Human light chains are typically classified as kappa and lambda light chains. Heavy chains are typically classified as mu, delta, gamma, alpha, or epsilon, and define the antibody's isotype as IgM, IgD, IgG, IgA, and IgE, respectively. IgG has several subclasses, including, but not limited to, IgG1, IgG2, IgG3, and IgG4. IgM has subclasses including, but not limited to, IgM1 and IgM2. IgA is similarly subdivided into subclasses including, but not limited to, IgAQ1 and IgA2. Within full-length light and heavy chains, typically, the variable and constant regions are joined by a “J” region of about 12 or more amino acids, with the heavy chain also including a “D” region of about 10 more amino acids. See, e.g., Fundamental Immunology, Ch. 7 (Paul, W., ed., 2nd ed. Raven Press, N.Y. (1989)) (incorporated by reference in its entirety for all purposes). The variable regions of each light/heavy chain pair typically form the antigen binding site.

The term “variable region” or “variable domain” refers to a portion of the light and/or heavy chains of an antibody, typically including approximately the amino-terminal 120 to 130 amino acids in the heavy chain and about 100 to 110 amino terminal amino acids in the light chain. In certain embodiments, variable regions of different antibodies differ extensively in amino acid sequence even among antibodies of the same species. The variable region of an antibody typically determines specificity of a particular antibody for its target.

The variable regions typically exhibit the same general structure of relatively conserved framework regions (FR) joined by three hyper variable regions, also called complementarity determining regions or CDRs. The CDRs from the two chains of each pair typically are aligned by the framework regions, which can enable binding to a specific epitope. From N-terminal to C-terminal, both light and heavy chain variable regions typically comprise the domains FR1, CDR1, FR2, CDR2, FR3, CDR3 and FR4. The assignment of amino acids to each domain is typically in accordance with the definitions of Kabat Sequences of Proteins of Immunological Interest (National Institutes of Health, Bethesda, Md. (1987 and 1991)), Chothia & Lesk, J. Mol. Biol., 196:901-917 (1987) or Chothia et al., Nature, 342:878-883 (1989).

In certain embodiments, an antibody heavy chain binds to an antigen in the absence of an antibody light chain. In certain embodiments, an antibody light chain binds to an antigen in the absence of an antibody heavy chain. In certain embodiments, an antibody binding region binds to an antigen in the absence of an antibody light chain. In certain embodiments, an antibody binding region binds to an antigen in the absence of an antibody heavy chain. In certain embodiments, an individual variable region specifically binds to an antigen in the absence of other variable regions.

Definitive delineation of a CDR and identification of residues comprising the binding site of an antibody may be accomplished by solving the structure of the antibody and/or solving the structure of the antibody-ligand complex, which can be accomplished by any of a variety of techniques known to those skilled in the art, such as X-ray crystallography. Various methods of analysis may be employed to identify or approximate the CDR regions. Examples of such methods include, but are not limited to, the Kabat definition, the Chothia definition, the AbM definition and the contact definition.

The Kabat definition is a standard for numbering the residues in an antibody and is typically used to identify CDR regions. See, e.g., Johnson & Wu, Nucleic Acids Res., 28: 214-8 (2000). The Chothia definition is similar to the Kabat definition, but the Chothia definition takes into account positions of certain structural loop regions. See, e.g., Chothia et al., J. Mol. Biol., 196: 901-17 (1986); Chothia et al., Nature, 342: 877-83 (1989). The AbM definition uses an integrated suite of computer programs produced by Oxford Molecular Group that model antibody structure. See, e.g., Martin et al., Proc Natl Acad Sci (USA), 86:9268-9272 (1989); “AbM™, A Computer Program for Modeling Variable Regions of Antibodies,” Oxford, UK; Oxford Molecular, Ltd. The AbM definition models the tertiary structure of an antibody from primary sequence using a combination of knowledge databases and ab initio methods, such as those described by Samudrala et al., “Ab Initio Protein Structure Prediction Using a Combined Hierarchical Approach,” in PROTEINS, Structure, Function and Genetics Suppl., 3:194-198 (1999). The contact definition is based on an analysis of the available complex crystal structures. See, e.g., MacCallum et al., J. Mol. Biol., 5:732-45 (1996).

By convention, the CDR regions in the heavy chain are typically referred to as H1, H2, and H3 and are numbered sequentially in the direction from the amino terminus to the carboxy terminus. The CDR regions in the light chain are typically referred to as L1, L2, and L3 and are numbered sequentially in the direction from the amino terminus to the carboxy terminus.

The term “light chain” includes a full-length light chain and fragments thereof having sufficient variable region sequence to confer binding specificity. A full-length light chain includes a variable region domain, VL, and a constant region domain, CL. The variable region domain of the light chain is at the amino-terminus of the polypeptide. Light chains include kappa chains and lambda chains.

The term “heavy chain” includes a full-length heavy chain and fragments thereof having sufficient variable region sequence to confer binding specificity. A full-length heavy chain includes a variable region domain, VH, and three constant region domains, CH1, CH2, and CH3. The VH domain is at the amino-terminus of the polypeptide, and the CH domains are at the carboxyl-terminus, with the CH3 being closest to the carboxy-terminus of the polypeptide. Heavy chains can be of any isotype, including IgG (including IgG1, IgG2, IgG3 and IgG4 subtypes), IgA (including IgA1 and IgA2 subtypes), IgM and IgE.

A bispecific or bifunctional antibody typically is an artificial hybrid antibody having two different heavy/light chain pairs and two different binding sites. Bispecific antibodies can be produced by a variety of methods including, but not limited to, fusion of hybridomas or linking of Fab′ fragments. See, e.g., Songsivilai et al., Clin. Exp. Immunol., 79: 315-321 (1990); Kostelny et al., J. Immunol., 148:1547-1553 (1992).

The term “antigen” refers to a substance capable of inducing adaptive immune responses. Specifically, an antigen is a substance which serves as a target for the receptors of an adaptive immune response. Typically, an antigen is a molecule that binds to antigen-specific receptors but cannot induce an immune response in the body by itself. Antigens are usually proteins and polysaccharides, less frequently also lipids. As used herein, antigens also include immunogens and haptens.

An “Fc” region comprises two heavy chain fragments comprising the CH1 and CH2 domains of an antibody. The two heavy chain fragments are held together by two or more disulfide bonds and by hydrophobic interactions of the CH3 domains.

The “Fv region” comprises the variable regions from both the heavy and light chains but lacks the constant regions.

An antibody that “specifically binds to” or is “specific for” a particular polypeptide or an epitope on a particular polypeptide is one that binds to that particular polypeptide or epitope on a particular polypeptide without substantially binding to any other polypeptide or polypeptide epitope. For example, the HCMV gB protein specific antibodies of the present disclosure are specific to HCMV gB protein. The antibody that binds to HCMV gB protein may have a dissociation constant (Kd) of ≤100 nM, ≤10 nM, ≤1 nM, ≤0.1 nM, ≤0.01 nM, or ≤0.001 nM (e.g., 10⁻⁸M or less, e.g., from 10⁻⁸M to 10⁻¹³M, e.g., from 10⁻⁹M to 10⁻¹³ M).

The term “compete” when used in the context of antigen binding proteins (e.g., atnibody or antigen-binding fragment thereof) that compete for the same epitope means competition between antigen binding proteins as determined by an assay in which the antigen binding protein (e.g., antibody or antigen-binding fragment thereof) being tested prevents or inhibits (e.g., reduces) specific binding of a reference antigen binding protein (e.g., a ligand, or a reference antibody) to a common antigen (e.g., HCMV gB protein or a fragment thereof). Numerous types of competitive binding assays can be used to determine if one antigen binding protein competes with another, for example: solid phase direct or indirect radioimmunoassay (RIA), solid phase direct or indirect enzyme immunoassay (EIA), sandwich competition assay (see, e.g., Stahli et al., 1983, Methods in Enzymology 9:242-253); solid phase direct biotin-avidin EIA (see, e.g., Kirkland et al., 1986, J. Immunol. 137:3614-3619) solid phase direct labeled assay, solid phase direct labeled sandwich assay (see, e.g., Harlow and Lane, 1988, Antibodies, A Laboratory Manual, Cold Spring Harbor Press); solid phase direct label RIA using 1-125 label (see, e.g., Morel et al., 1988, Molec. Immunol. 25:7-15); solid phase direct biotin-avidin EIA (see, e.g., Cheung, et al., 1990, Virology 176:546-552); and direct labeled RIA (Moldenhauer et al., 1990, Scand. J. Immunol. 32:77-82). Typically, such an assay involves the use of purified antigen bound to a solid surface or cells bearing either of these, an unlabelled test antigen binding protein and a labeled reference antigen binding protein. Competitive inhibition is measured by determining the amount of label bound to the solid surface or cells in the presence of the test antigen binding protein. Usually, the test antigen binding protein is present in excess. Antigen binding proteins identified by competition assay (competing antigen binding proteins) include antigen binding proteins binding to the same epitope as the reference antigen binding proteins and antigen binding proteins binding to an adjacent epitope sufficiently proximal to the epitope bound by the reference antigen binding protein for steric hindrance to occur. Additional details regarding methods for determining competitive binding are provided in the examples herein. Usually, when a competing antigen binding protein is present in excess, it will inhibit (e.g., reduce) specific binding of a reference antigen binding protein to a common antigen by at least 40-45%, 45-50%, 50-55%, 55-60%, 60-65%, 65-70%, 70-75% or 75% or more. In some instances, binding is inhibited by at least 80-85%, 85-90%, 90-95%, 95-97%, or 97% or more.

The term “epitope” as used herein refers to the specific group of atoms or amino acids on an antigen to which an antibody binds. The epitope can be either linear epitope or a conformational epitope. A linear epitope is formed by a continuous sequence of amino acids from the antigen and interacts with an antibody based on their primary structure. A conformational epitope, on the other hand, is composed of discontinuous sections of the antigen's amino acid sequence and interacts with the antibody based on the 3D structure of the antigen. In general, an epitope is approximately five or six amino acids in length. Two antibodies may bind the same epitope within an antigen if they exhibit competitive binding for the antigen.

The term “host cell” means a cell that has been transformed, or is capable of being transformed, with a nucleic acid sequence and thereby expresses a gene of interest. The term includes the progeny of the parent cell, whether or not the progeny is identical in morphology or in genetic make-up to the original parent cell, so long as the gene of interest is present.

The term “identity” refers to a relationship between the sequences of two or more polypeptide molecules or two or more nucleic acid molecules, as determined by aligning and comparing the sequences. “Percent identity” means the percent of identical residues between the amino acids or nucleotides in the compared molecules and is calculated based on the size of the smallest of the molecules being compared. For these calculations, gaps in alignments (if any) are preferably addressed by a particular mathematical model or computer program (i.e., an “algorithm”). Methods that can be used to calculate the identity of the aligned nucleic acids or polypeptides include those described in Computational Molecular Biology, (Lesk, A. M., ed.), 1988, New York: Oxford University Press; Biocomputing Informatics and Genome Projects, (Smith, D. W., ed.), 1993, New York: Academic Press; Computer Analysis of Sequence Data, Part I, (Griffin, A. M., and Griffin, H. G., eds.), 1994, New Jersey: Humana Press; von Heinje, G., 1987, Sequence Analysis in Molecular Biology, New York: Academic Press; Sequence Analysis Primer, (Gribskov, M. and Devereux, J., eds.), 1991, New York: M. Stockton Press; and Carillo et al., 1988, SIAM J. Applied Math. 48:1073.

In calculating percent identity, the sequences being compared are typically aligned in a way that gives the largest match between the sequences. One example of a computer program that can be used to determine percent identity is the GCG program package, which includes GAP (Devereux et al., 1984, Nucl. Acid Res. 12:387; Genetics Computer Group, University of Wisconsin, Madison, Wis.). The computer algorithm GAP is used to align the two polypeptides or polynucleotides for which the percent sequence identity is to be determined. The sequences are aligned for optimal matching of their respective amino acid or nucleotide (the “matched span”, as determined by the algorithm). A gap opening penalty (which is calculated as 3× the average diagonal, wherein the “average diagonal” is the average of the diagonal of the comparison matrix being used; the “diagonal” is the score or number assigned to each perfect amino acid match by the particular comparison matrix) and a gap extension penalty (which is usually 1/10 times the gap opening penalty), as well as a comparison matrix such as PAM 250 or BLOSUM 62 are used in conjunction with the algorithm. A standard comparison matrix (see, Dayhoff et al., 1978, Atlas of Protein Sequence and Structure 5:345-352 for the PAM 250 comparison matrix; Henikoff et al., 1992, Proc. Natl. Acad. Sci. U.S.A. 89:10915-10919 for the BLOSUM 62 comparison matrix) may be also used by the algorithm.

Examples of parameters that can be employed in determining percent identity for polypeptides or nucleotide sequences using the GAP program can be found in Needleman et al., 1970, J. Mol. Biol. 48:443-453.

Certain alignment schemes for aligning two amino acid sequences may result in matching of only a short region of the two sequences, and this small aligned region may have very high sequence identity even though there is no significant relationship between the two full-length sequences. Accordingly, the selected alignment method (GAP program) can be adjusted if so desired to result in an alignment that spans at least 50 or other number of contiguous amino acids of the target polypeptide.

The term “link” as used herein refers to the association via intramolecular interaction, e.g., covalent bonds, metallic bonds, and/or ionic bonding, or inter-molecular interaction, e.g., hydrogen bond or noncovalent bonds.

The term “operably linked” refers to an arrangement of elements wherein the components so described are configured so as to perform their usual function. Thus, a given signal peptide that is operably linked to a polypeptide directs the secretion of the polypeptide from a cell. In the case of a promoter, a promoter that is operably linked to a coding sequence will direct the expression of the coding sequence. The promoter or other control elements need not be contiguous with the coding sequence, so long as they function to direct the expression thereof. For example, intervening untranslated yet transcribed sequences can be present between the promoter sequence and the coding sequence and the promoter sequence can still be considered “operably linked” to the coding sequence.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

The term “polynucleotide” or “nucleic acid” includes both single-stranded and double-stranded nucleotide polymers. The nucleotides comprising the polynucleotide can be ribonucleotides or deoxyribonucleotides or a modified form of either type of nucleotide. Said modifications include base modifications such as bromouridine and inosine derivatives, ribose modifications such as 2′,3′-dideoxyribose, and intemucleotide linkage modifications such as phosphorothioate, phosphorodithioate, phosphoroselenoate, phosphorodiselenoate, phosphoroanilothioate, phoshoraniladate and phosphoroamidate.

The terms “polypeptide” or “protein” means a macromolecule having the amino acid sequence of a native protein, that is, a protein produced by a naturally-occurring and non-recombinant cell; or it is produced by a genetically-engineered or recombinant cell, and comprise molecules having the amino acid sequence of the native protein, or molecules having deletions from, additions to, and/or substitutions of one or more amino acids of the native sequence. The term also includes amino acid polymers in which one or more amino acids are chemical analogs of a corresponding naturally occurring amino acid and polymers. The terms “polypeptide” and “protein” specifically encompass HCMV gB protein binding proteins, antibodies, or sequences that have deletions from, additions to, and/or substitutions of one or more amino acid of antigen-binding protein. The term “polypeptide fragment” refers to a polypeptide that has an amino-terminal deletion, a carboxyl-terminal deletion, and/or an internal deletion as compared with the full-length native protein. Such fragments can also contain modified amino acids as compared with the native protein. Fragments may be about five to 500 amino acids long. For example, fragments can be at least 5, 6, 8, 10, 14, 20, 50, 70, 100, 110, 150, 200, 250, 300, 350, 400, or 450 amino acids long. Useful polypeptide fragments include immunologically functional fragments of antibodies, including binding domains. In the case of a HCMV gB protein-binding antibody, useful fragments include but are not limited to a CDR region, a variable domain of a heavy and/or light chain, a portion of an antibody chain or just its variable region including two CDRs, and the like.

The pharmaceutically acceptable carriers are conventional. Remington's Pharmaceutical Sciences, by E. W. Martin, Mack Publishing Co., Easton, PA, 15th Edition (1975), describes compositions and formulations suitable for pharmaceutical delivery of the fusion proteins herein disclosed. In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (e.g., powder, pill, tablet, or capsule forms), conventional non-toxic solid carriers can include, for example, pharmaceutical grades of mannitol, lactose, starch or magnesium stearate. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

As used herein, the term “subject” refers to a human or any non-human animal (e.g., mouse, rat, rabbit, dog, cat, cattle, swine, sheep, horse or primate). A human includes pre- and post-natal forms. A subject may be a human being. A subject can be a patient, which refers to a human presenting to a medical provider for diagnosis or treatment of a disease. The term “subject” is used herein interchangeably with “individual” or “patient.” A subject can be afflicted with or is susceptible to a disease or disorder but may or may not display symptoms of the disease or disorder.

The term “therapeutically effective amount” or “effective dosage” as used herein refers to the dosage or concentration of a drug effective to treat a disease or condition. For example, with regard to the use of the monoclonal antibodies or antigen-binding fragments thereof disclosed herein to treat viral infection.

“Treating” or “treatment” of a condition as used herein includes preventing or alleviating a condition, slowing the onset or rate of development of a condition, reducing the risk of developing a condition, preventing or delaying the development of symptoms associated with a condition, reducing or ending symptoms associated with a condition, generating a complete or partial regression of a condition, curing a condition, or some combination thereof.

As used herein, a “vector” refers to a nucleic acid molecule as introduced into a host cell, thereby producing a transformed host cell. A vector may include nucleic acid sequences that permit it to replicate in the host cell, such as an origin of replication. A vector may also include one or more therapeutic genes and/or selectable marker genes and other genetic elements known in the art. A vector can transduce, transform or infect a cell, thereby causing the cell to express nucleic acids and/or proteins other than those native to the cell. A vector optionally includes materials to aid in achieving entry of the nucleic acid into the cell, such as a viral particle, liposome, protein coating or the like.

### IX. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

The base construct used for the HCMV gB variant contained residues 1-704 of HCMV gB Towne strain (SEQ ID NO: 1) with serine substituted at residues 246, 457 and 460 to remove an unpaired, non-conserved cysteine and the furin cleavage site (Burke et al., 2015; Nelson et al., 2020). The foldon trimerization motif of T4 fibritin (Fd) was included after Domain V (P704). The construct further included an HRV3C protease recognition site, an octa-histidine tag, and a tandem Twin-Strep-tag, cloned into the mammalian expression plasmid paH. All mutations in subsequent designs were introduced into this base construct, which is provided as SEQ ID NO: 4. In some constructs, a hexa-histidine tag and a singular Strep-tag are included instead of the octa-histidine tag and Twin-Strep-tag from the base construct, and the HRV3C protease recognition site is not included.

Using structure-based design, substitutions that were aimed at favoring the stability of the prefusion structure were introduced into the base construct. Pairs of core-facing residues less than 5 Å apart were replaced with aromatic sidechains or pairs of aromatic and positively charged sidechains to favor pi-pi or pi-cation interactions, respectively. Alternatively, residues were replaced with extended or bulkier hydrophobic sidechains in efforts to fill pre-existing internal cavities. Disulfide bonds were designed to increase overall stability or prevent formation of the postfusion conformation. The charged or polar substitutions were aimed to establish hydrogen bonds or salt bridges with the native residues that were predicted to be within 4.0 Å. These were then combined with individual substitutions or combinations of substitutions from the structure-based designs that were shown to be beneficial.

In addition, apex designs were generated with the goal of replacing flexible hinge region (residues ˜S438-L484) with a helix cap. In S2PA (i.e., Apxl), L439-Q483 was replaced by the sequence RLNPP (SEQ ID NO: 78). In Capla (i.e., Apx2), L439-Q483 was replaced by the sequence GADPQ (SEQ ID NO: 79). In Apx3, L439-L484 was replaced by the sequence GMTPEQ (SEQ ID NO: 80). In Apx4, K437-F486 was replaced by the sequence YDVCDPQLCY (SEQ ID NO: 81). In Apx5, L439-T487 was replaced by the sequence VGPPPTHKI (SEQ ID NO: 82). As another strategy, a larger region around the flexible hinge region was removed (residues Q436-D489) and replaced with a short linker. In CL-25, Q436-D489 was replaced by the sequence GGP. In CL-26, Q436-D489 was replaced by the sequence GPP. In CL-27, Q436-D489 was replaced by the sequence PGP. In RMc1, Q436-D489 was replaced by the sequence GPG.

In addition, different trimerization motifs were tested. For example, the foldon present in the base construct was replaced by a modified PIV5/GCN4 hybrid or a modified Cortexillin C-term (4J4A). For the modified GCN4 construct, the foldon trimerization motif was removed, along with residues 694-704 of gB (i.e., positions V694-P746 of SEQ ID NO: 4). In their place, the sequence 694-DTYLSAIEDKIEEILSKIYHIENEIARI-721 (SEQ ID NO: 83) was inserted. For the modified 4J4A construct, the foldon trimerization motif was removed, along with residues 690-704 of gB (i.e., positions Y690-P746 of SEQ ID NO: 4). In their place, the sequence 690-LKQIVLRIMEIEARIAKIE-708 (SEQ ID NO: 86) was inserted. A construct with a combined 4J4A+Foldon was also generated, where the foldon trimerization motif was removed, along with residues 690-704 of gB (i.e., positions Y690-P746 of SEQ ID NO: 4), and the sequence 690-LKQIVLRIMEIEARIAKIEGSGYIPEAPRDGQAYVRKDGEWVLLSTFLG-738 (SEQ ID NO: 87) was inserted in their place. Additional constructs with a combined 4J4A+4J4A were also generated, where the foldon trimerization motif was removed, along with residues 690-704 of gB (i.e., position Y690-P746 of SEQ ID NO: 4), and the sequence 690-LKQIVLRIMEIEARIAKIEGSEFNSLKQIVLRIMEIEARIAKIE-738 (SEQ ID NO: 88), 690-LKQIVLRIMEIEARIAKIEGSLKQIVLRIMEIEARIAKIE-729 (SEQ ID NO: 89), or 690-LKQIVLRIMEIEARIAKIEGSLELIKLRIMEIEARIAKIEKDRAIL-735 (SEQ ID NO: 90) was inserted in their place.

Exemplary substitutions and sets of substitutions are provided in Tables 1 and 2.

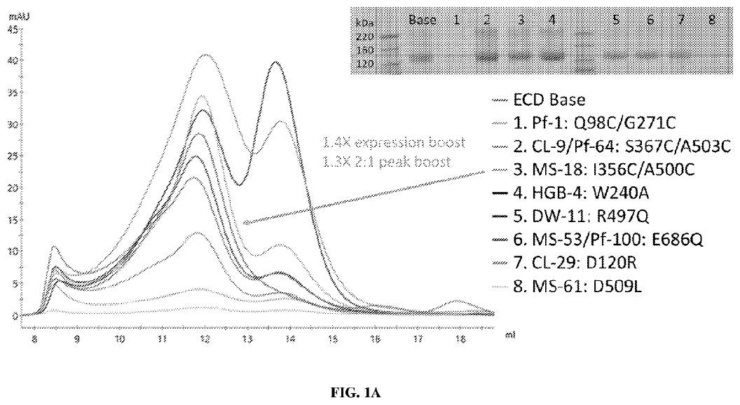

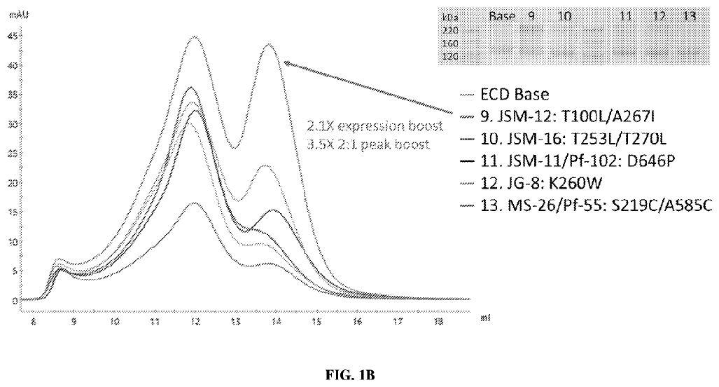

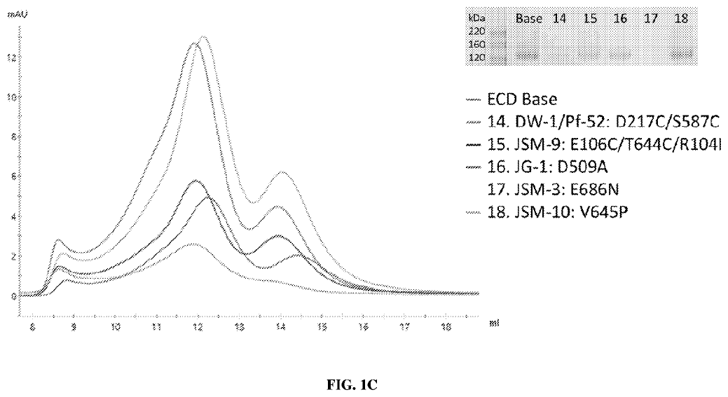

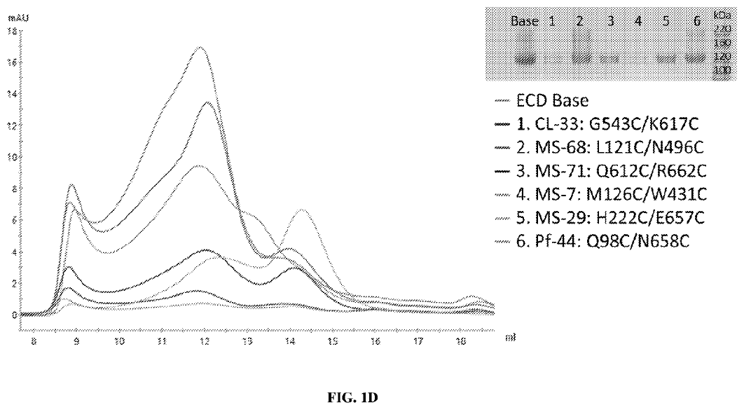

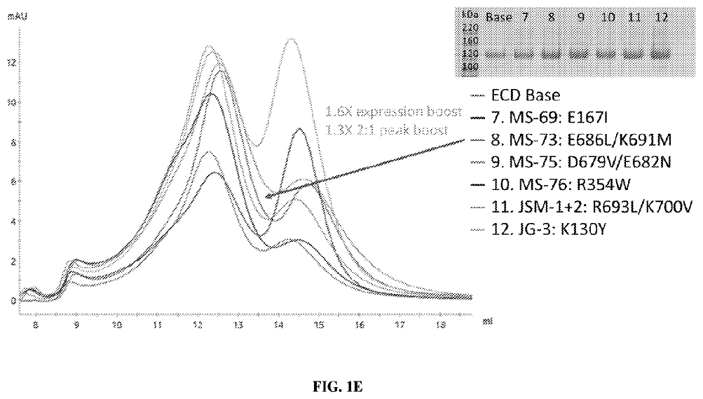

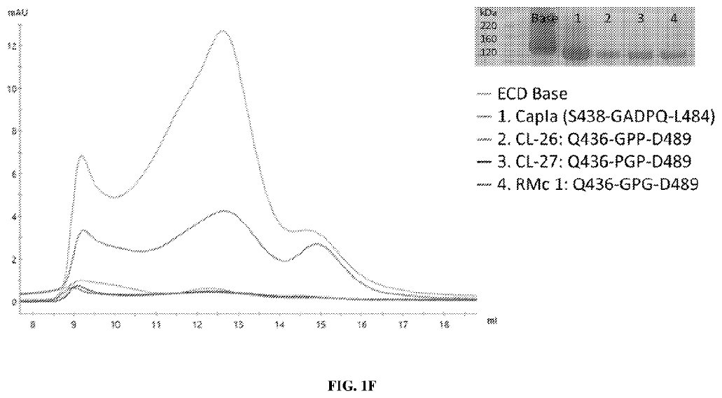

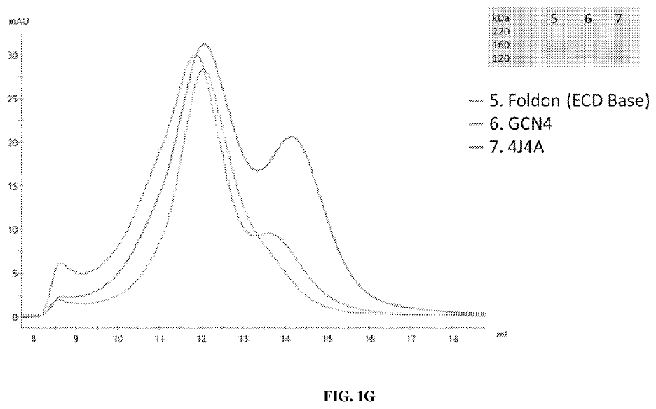

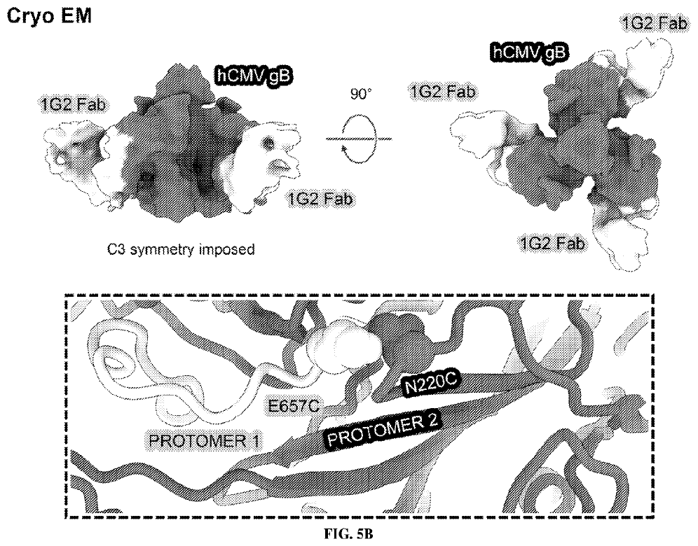

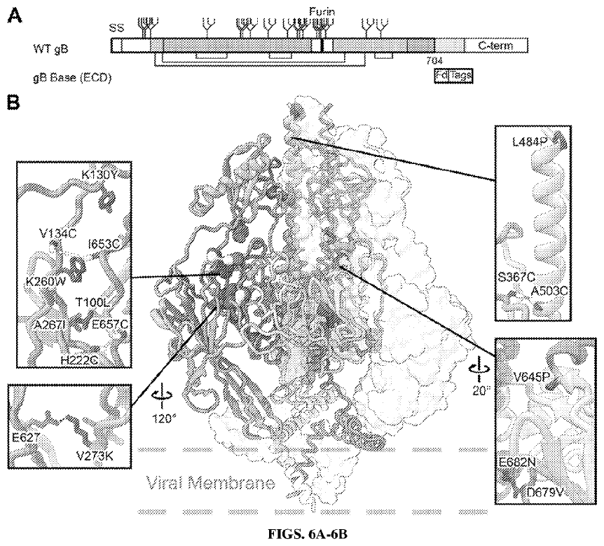

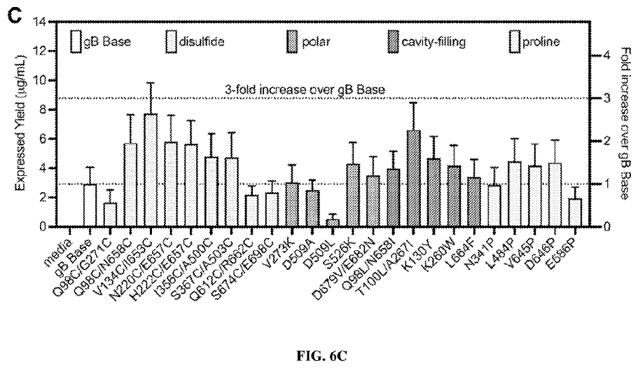

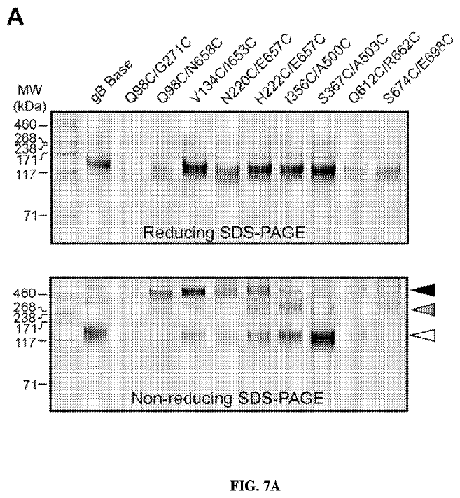

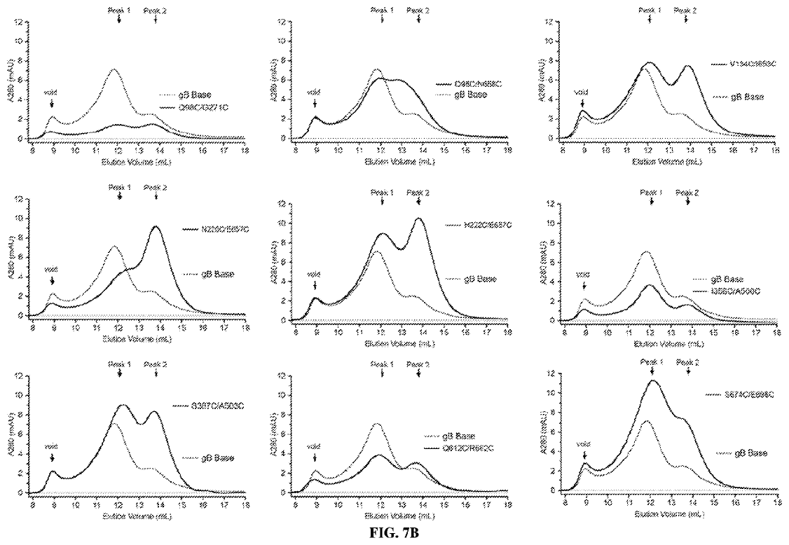

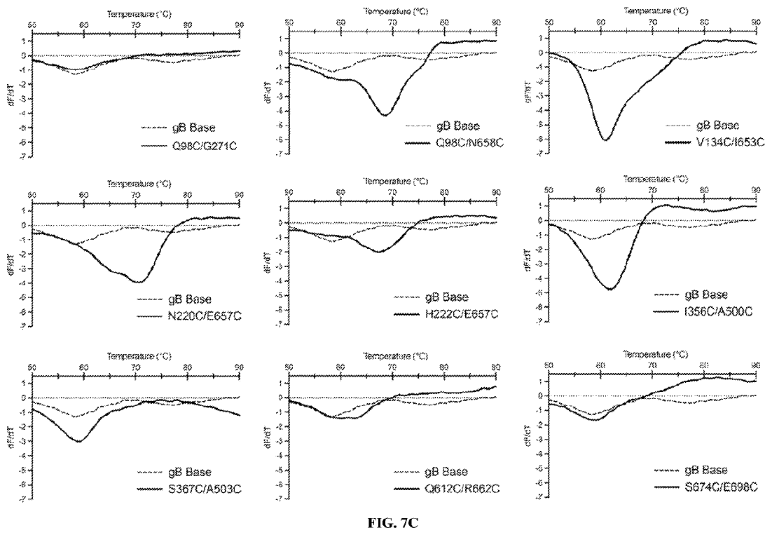

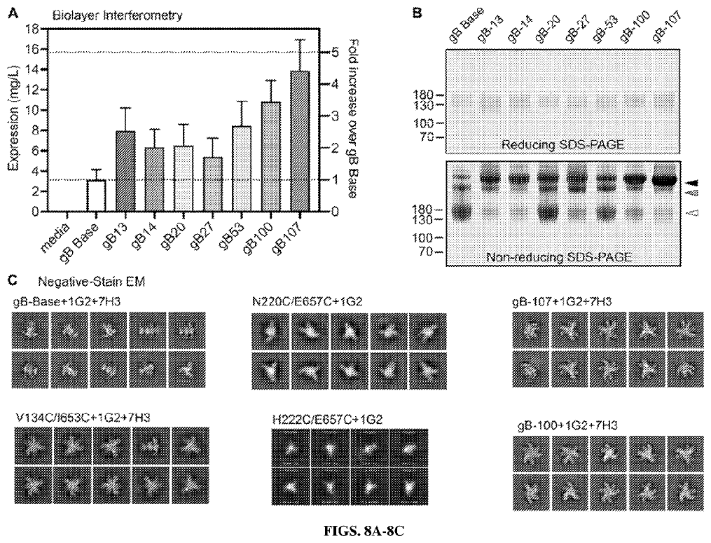

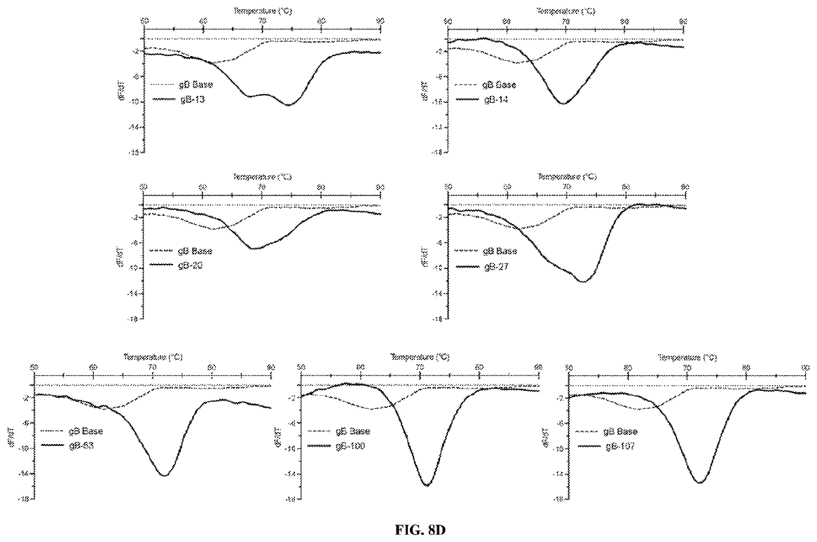

Plasmids encoding HCMV gB variants were transiently transfected into FreeStyle293F cells (Thermo Fisher) using polyethyleneimine, with 5 pM kifunensine being added 3 h post-transfection. Cultures were grown for 4-6 days, and culture supernatant was separated via centrifugation and passage through a 0.22 μm filter. Protein was purified from supernatants using StrepTactin resin (IBA). HCMV gB variants were further purified by size-exclusion chromatography (SEC) using a Superose 6 10/300 column (GE Healthcare) in a buffer composed of 2 mM Tris pH 8.0, 200 mM NaCl and 0.02% NaN3. For initial purification and characterization, single-substitution and combinatorial variants were purified from 40 mL cultures. While it is expected that the N-terminal signal sequence (i.e., amino acids 1-22 of SEQ ID NO: 4) is cleaved during ER translocation, and thus absent from the mature protein, the affinity tags at the C-terminus of the molecules were included in structural and biophysical analysis, with the exception that in the cryo-EM studies of gB-107, the affinity tags were cleaved using HRV-3C, such that the C-terminus of the protein corresponds to Q744 of SEQ ID NO: 53. The protein purity, monodispersity and expression level were determined by SDS-PAGE and SEC. Exemplary graph traces and gels are provided in FIGS. 1A-1G, as well as in FIGS. 7A, 7B and 8B. In the graph graces, the first peak corresponds to multimers of trimeric post-fusion gB; the second peak corresponds to monomeric gB trimers. Further data obtained from the SEC evaluation is provided in Tables 3 and 4. Graphical representations of the expressed yield are provided in FIGS. 6C and 8A.

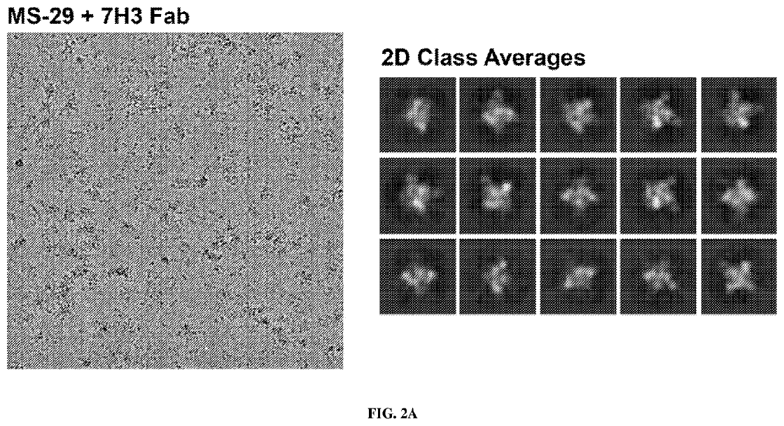

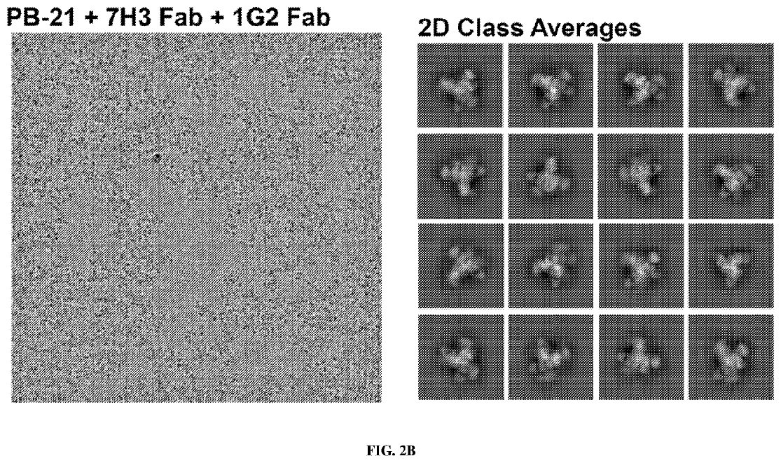

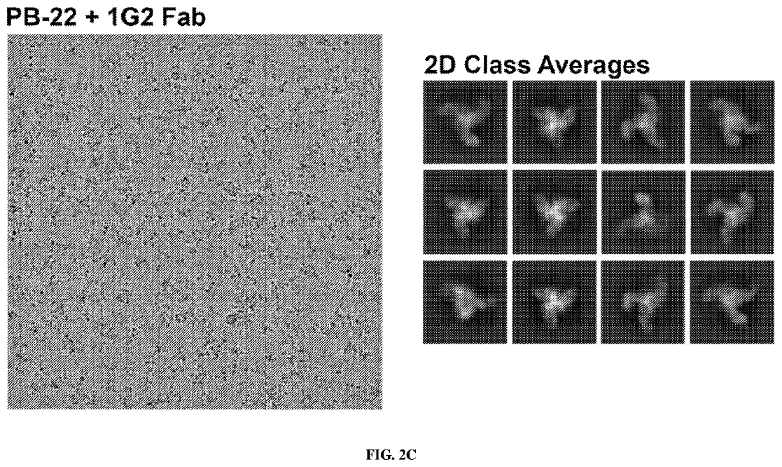

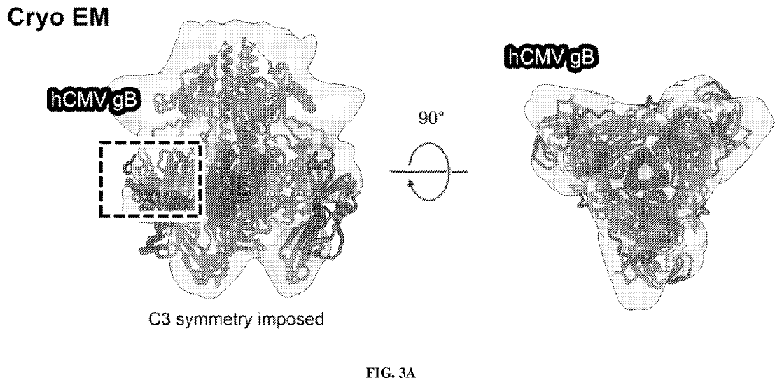

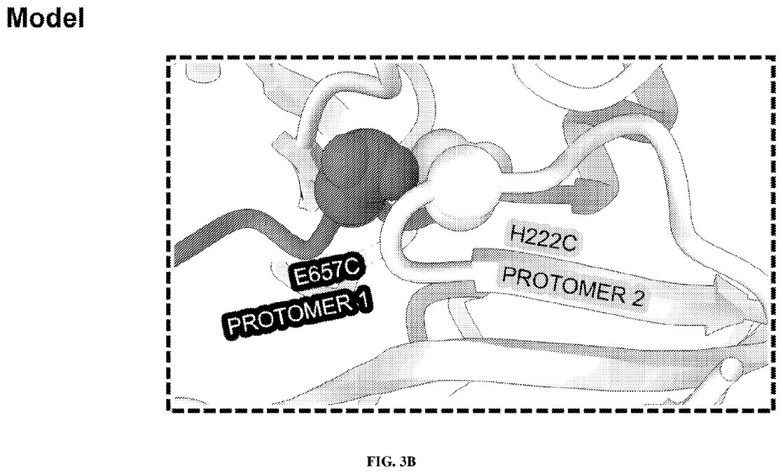

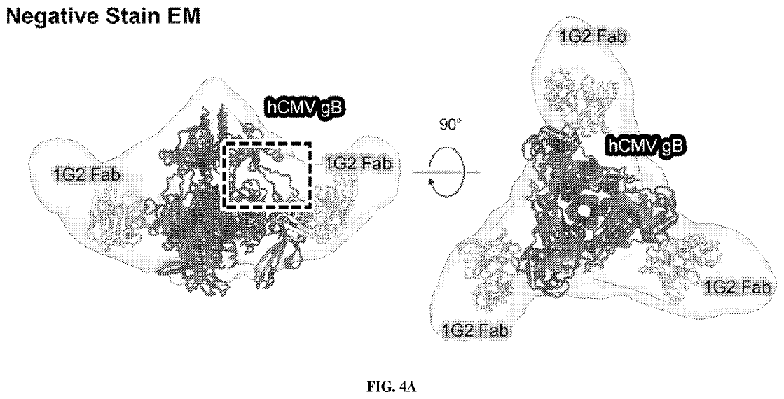

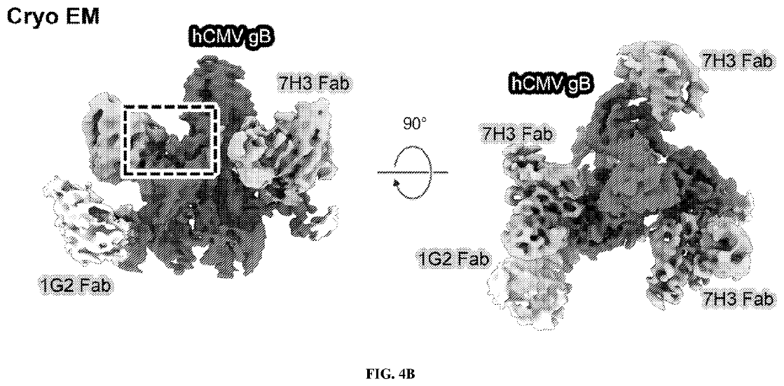

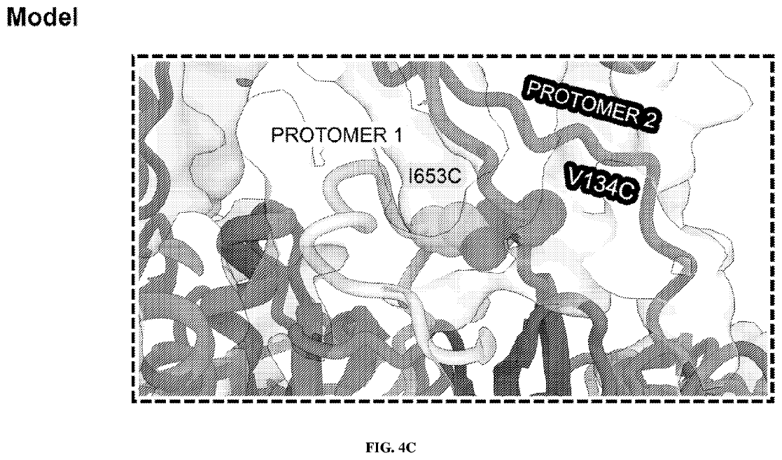

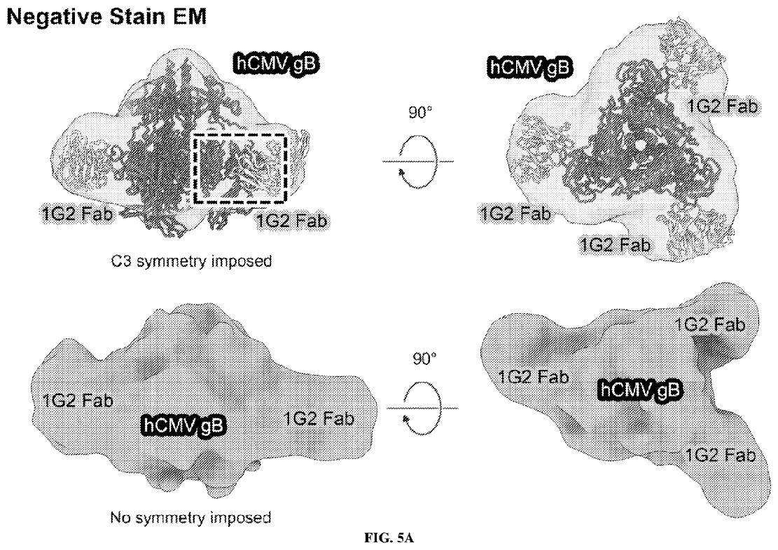

Negative stain electron microscopy (nsEM) analysis was performed on some of the HCMV gB variants. Purified HCMV gB variants were diluted to a concentration of 0.06 mg/mL in 2 mM Tris pH 8.0, 200 mM NaCl and 0.02% NaN₃. Each protein was deposited on a CF-400-CU grid (Electron Microscopy Sciences) that had been plasma cleaned for 30 seconds in a Solarus 950 plasma cleaner (Gatan) with a 4:1 ratio of O₂/H₂ and stained using methylamine tungstate (Nanoprobes). Grids were imaged at a magnification of 60,000× (corresponding to a calibrated pixel size of 3.6 Å/pix) in a Talos F200C TEM microscope equipped with a Ceta 16M detector (Thermo Fisher Scientific). Exemplary images are provided in FIGS. 2A-2D and 8C.

Differential scanning fluorimetry. Purified HCMV gB variants at a final concentration of 0.6 mg/mL were mixed with a final concentration 15×SYPRO Orange Protein Gel Stain (ThermoFisher) in a white, opaque 96-well plate (VWR). The solutions were then measured by continuous fluorescence scanning (λex=465 nm, λem=580 nm) using a Roche LightCycler 480 II, with a temperature ramp rate of 4.4° C./minute, and a temperature range of 25° C. to 95° C. Data were plotted as the first derivative of the fluorescence with respect to temperature (dF/dT) as a function of temperature. Data for individual designs are presented in FIG. 7C. Data for combined designs are presented in FIG. 8D.

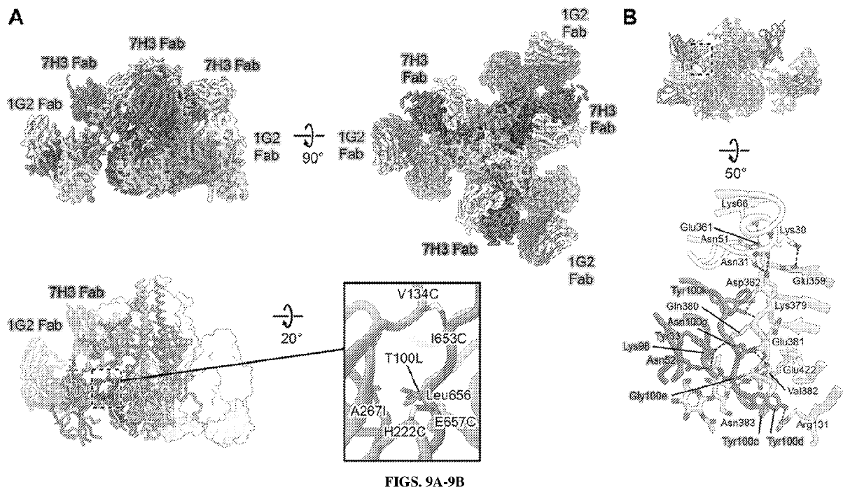

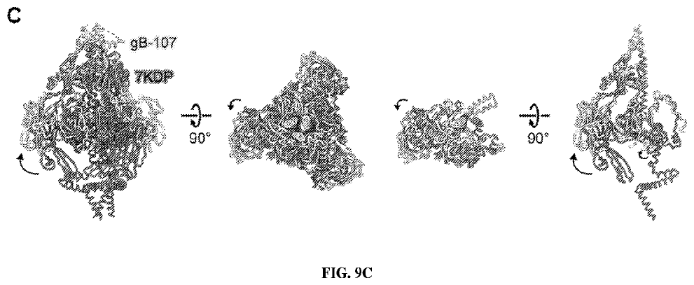

Cryo-EM sample structure of HCMV gB-107. Purified HCMV gB-107 was diluted to a concentration of 4.0 mg/mL and mixed with 7H3 and 1G2 Fabs at approximately equimolar concentrations. The buffer was 2 mM Tris pH 8.0, 200 mM NaCl, 2.5 mM CHAPS, 3% glycerol (v/v) and 0.02% (w/v) NaN₃. The sample was applied to glow-discharged C-Flat 1.2/1.3 grids (Protochips) before being blotted for 5 seconds in a Vitrobot Mark IV (ThermoFisher) and plunge frozen into liquid ethane. Micrographs were collected from a single grid using a Titan Krios (ThermoFisher) equipped with a K3 direct electron detector (Gatan). Data were collected at a magnification of 105,000×, corresponding to a calibrated pixel size of 0.84 Å/pix. The cryo-EM structure of HCMV gB-107 was determined at a resolution of 2.8 Å (FIGS. 9A-9C). HCMV gB-107 adopts the prefusion conformation, stabilized by the T100L/A267I, V134C/I653C, and H22C/E657C substitutions, which tether structural domain I to structural domains IV and V via hydrophobic packing and disulfide bonding respectively (FIG. 9A). Similar to the previously determined 1G2-bound HCMV gB structure (PDB accession code 5C6T), 1G2 binds a conformational epitope in antigenic domain 5 (AD-5) on structural domain I of gB-107 (FIG. 9A). We also modeled the interface between HCMV gB-107 and Fab 7H3 (FIG. 9B). Somewhat similar to the SM5-1-bound HCMV gB structure (PDB accession code 7KDD), 7H3 binds AD-4 on domain II. Taken together, this demonstrates that the stabilizing substitutions tethering domain I to domains IV and V have a negligible effect on the native fold of domains I and II. Furthermore, the structure of the soluble HCMV gB-107 ectodomain closely resembles the published prefusion structure of full length HCMV gB (PDB accession code 7KDP) (FIG. 9C), confirming that the stabilizing substitutions do not alter the native-like prefusion conformation of this soluble ectodomain construct. In the published prefusion structure of full length HCMV gB, domain I is anchored by the membrane proximal region through hydrophobic interactions with the fusion loops at the membrane-proximal tips of domain I (Liu et al., 2021; PDB accession code 7KDP). Shifts in domain arrangement indicated by arrows in FIG. 9C likely occur due to flexing of domain I in the absence of the MPR, which is excluded in our soluble ectodomain construct along with the transmembrane domain and C-terminal domain by truncating at residue 704 (FIG. 6A).

TABLE 3
SEC Evaluation of Individual Designs

| | | | | Normalized | |
|---|---|---|---|---|---|
| | | Normalized | Normalized | Expression | Peak 2 |
| Variant | | Peak 1 | Peak 2 | (Peak 1 + | AUC/Peak |
| ID | Mutation(s) | AUC | AUC | Peak 2) | 1 AUC |
| PB-4 | Y89C/G622C | 0.31 | 0.54 | 0.36 | 1.76 |
| MS-33 | A97C/A549C | 0.61 | 0.88 | 0.66 | 1.45 |
| Pf-1 | Q98C/G271C | 0.19 | 0.41 | 0.22 | 2.20 |
| Pf-44 | Q98C/N658C | 0.52 | 1.04 | 0.63 | 1.99 |
| PB-1 | G99C/L616C | 0.16 | 0.28 | 0.18 | 1.81 |
| PB-12 | R104C/I642C | 0.62 | 0.77 | 0.77 | 1.24 |
| JSM-9 | E106C/T644C/R104I | 0.44 | 0.70 | 0.49 | 1.59 |
| MS-68 | L121C/N496C | 0.74 | 0.78 | 0.78 | 1.06 |
| MS-7 | M126C/W431C/R357W | 0.04 | 0.12 | 0.06 | 2.85 |
| PB-21 | V134C/I653C | 1.34 | 2.93 | 1.79 | 2.18 |
| PB-20 | A135C/I653C | 1.19 | 3.14 | 1.58 | 2.64 |
| MS-4 | Y160C/Y708C | 1.13 | 3.62 | 1.83 | 3.20 |
| DW-1 | D217C/S587C | 0.22 | 0.12 | 0.20 | 0.57 |
| MS-26 | S219C/A585C | 0.54 | 0.63 | 0.56 | 1.15 |
| PB-22 | N220C/E657C | 0.49 | 3.58 | 1.13 | 7.24 |
| MS-29 | H222C/E657C | 0.68 | 2.20 | 0.93 | 3.23 |
| PB-17 | K260C/S641C | 0.56 | 1.10 | 0.67 | 1.96 |
| PB-13 | V273C/L616C | 0.22 | 0.32 | 0.25 | 1.46 |
| PB-18 | T347C/A650C | 0.74 | 0.99 | 0.79 | 1.34 |
| Pf-68 | W349C/A650C | 0.77 | 1.23 | 0.87 | 1.59 |
| MS-18 | I356C/A500C | 1.31 | 1.72 | 1.37 | 1.32 |
| Pf-70 | S367C/A503C | 1.96 | 4.81 | 2.42 | 2.46 |
| PB-6 | M371C/A505C | 0.83 | 1.67 | 1.00 | 2.01 |
| CL-33 | G543C/K617C | 0.07 | 0.11 | 0.08 | 1.70 |
| PB-9 | D544C/Q597C | 0.11 | 0.25 | 0.13 | 2.34 |
| PB-8 | D544C/I620C | 0.13 | 0.28 | 0.16 | 2.12 |
| PB-19 | V545C/I618C | 0.04 | 0.08 | 0.04 | 2.30 |
| Pf-80 | A549C/N658C | 0.96 | 1.30 | 1.06 | 1.36 |
| Pf-81 | S550C/P655C | 0.71 | 0.83 | 0.75 | 1.16 |
| Pf-82 | S550C/E657C | 0.57 | 1.09 | 0.68 | 1.92 |
| MS-71 | Q612C/R662C | 0.23 | 0.56 | 0.30 | 2.44 |
| PB-5 | G604C/Y667C | 0.19 | 0.24 | 0.20 | 1.27 |
| Pf-85 | G604C/L672C | 0.08 | 0.27 | 0.12 | 3.19 |
| CL-35 | T644C/S647C | 0.78 | 1.49 | 0.91 | 1.93 |
| PB-16 | F661C/Y218C | 1.48 | 2.07 | 1.60 | 1.40 |
| MS-50 | S674C/E698C | 2.27 | 1.42 | 2.11 | 0.63 |
| PB-14 | E698C/R673C | 1.29 | 1.47 | 1.33 | 1.14 |
| MS-80 | Q98L/N658I | 0.98 | 2.35 | 1.25 | 2.39 |
| JSM-12 | T100L/A267I | 1.44 | 5.00 | 2.06 | 3.47 |
| CL-41 | L102W | 1.50 | 3.19 | 1.81 | 2.13 |
| MS-79 | I103Y | 1.36 | 1.17 | 1.31 | 0.86 |
| JG-4 | K130L | 1.51 | 3.86 | 2.33 | 2.55 |
| JG-3 | K130Y | 1.75 | 4.02 | 1.95 | 2.29 |
| MS-32 | T159W | 1.41 | 0.44 | 1.13 | 0.31 |
| MS-77 | L162W | 1.11 | 2.03 | 1.28 | 1.82 |
| JSM-13 | N220F/T659V | 0.65 | 0.66 | 0.65 | 1.01 |
| CL-47 | S223V/T253V/ | 0.97 | 1.46 | 1.06 | 1.51 |
| | T270M/T659V | | | | |
| JSM-16 | T253L/T270L | 1.09 | 1.15 | 1.10 | 1.05 |
| JG-8 | K260W | 1.11 | 2.53 | 1.36 | 2.27 |
| JSM-21 | V273F | 0.80 | 2.52 | 1.08 | 3.15 |
| DW-6 | L346Y | 0.93 | 0.83 | 0.91 | 0.89 |
| MS-76 | R354W | 1.62 | 1.00 | 1.46 | 0.62 |
| CL-37 | S367M | 1.54 | 2.01 | 1.63 | 1.30 |
| DW-10 | G493M | 0.72 | 0.89 | 0.75 | 1.23 |
| JSM-22 | A500L/A503S | 1.32 | 0.96 | 1.22 | 0.72 |
| MS-81 | R512W | 1.34 | 1.20 | 1.30 | 0.89 |
| CL-18 | F541W | 0.82 | 0.70 | 0.80 | 0.85 |
| CL-40 | L616W | 1.17 | 1.04 | 1.15 | 0.89 |
| JSM-19 | R607Y | 0.86 | 0.69 | 0.81 | 0.80 |
| CL-14 | V645L | 1.01 | 1.41 | 1.09 | 1.39 |
| MS-78 | R662W | 1.26 | 1.62 | 1.37 | 1.28 |
| JSM-15 | L664F | 1.54 | 1.92 | 1.61 | 1.25 |
| HGB-4 | W240A | 1.29 | 6.07 | 2.06 | 4.71 |
| MS-69 | E167I | 0.95 | 2.25 | 1.28 | 2.36 |
| JG-1 | D509A | 0.38 | 0.51 | 0.40 | 1.36 |
| MS-61 | D509L | 0.05 | 0.11 | 0.06 | 2.30 |
| MS-75 | D679V/E682N | 1.84 | 2.29 | 1.96 | 1.24 |
| MS-73 | E686L/K691M | 1.52 | 2.02 | 1.65 | 1.33 |
| MS-84 | E686M | 1.58 | 0.78 | 1.35 | 0.49 |
| JSM-1 + 2 | R693L/K700V | 1.83 | 1.82 | 1.83 | 1.00 |
| DW-11 | R497Q | 1.09 | 1.04 | 1.08 | 0.96 |
| MS-53 | E686Q | 1.16 | 0.69 | 1.08 | 0.59 |
| CL-29 | D120R | 0.57 | 0.58 | 0.57 | 1.01 |
| JSM-17 | L161E | 1.18 | 1.16 | 1.18 | 0.98 |
| JSM-20 | V273K | 1.61 | 1.66 | 1.62 | 1.03 |
| CL-8 | E359R | 1.52 | 1.16 | 1.45 | 0.76 |
| CL-43 | S526K | 0.94 | 1.30 | 1.01 | 1.38 |
| CL-38 | D630R | 0.03 | 0.04 | 0.03 | 1.57 |
| MS-25 | N341P | 2.03 | 1.53 | 1.94 | 0.75 |
| DW-9 | G433P | 1.00 | 1.24 | 1.05 | 1.24 |
| JSM-4 | H477P | 0.86 | 0.88 | 0.87 | 1.03 |
| JSM-5 | N478P | 1.02 | 1.02 | 1.02 | 1.00 |
| JSM-6 | L479P | 0.96 | 1.91 | 1.13 | 1.99 |
| JSM-7 | V480P | 1.23 | 2.39 | 1.43 | 1.95 |
| JSM-8 | Y481P | 1.23 | 2.24 | 1.41 | 1.83 |
| MS-36 | A482P | 1.34 | 1.25 | 1.32 | 0.93 |
| CL-36 | Q483P | 1.24 | 1.76 | 1.34 | 1.42 |
| MS-43 | L484P | 1.72 | 4.09 | 2.14 | 2.38 |
| MS-83 | E686P | 1.78 | 1.25 | 1.69 | 0.70 |
| JSM-10 | V645P | 0.95 | 2.03 | 1.17 | 2.14 |
| Pf-102 | D646P | 0.98 | 1.81 | 1.12 | 1.85 |
| CL-3 | A650P/L651P | 1.08 | 0.99 | 1.06 | 0.91 |
| 4A4J | Trimerization motif | 1.02 | 2.81 | 1.33 | 2.76 |
| GCN4 | Trimerization motif | 0.74 | 0.66 | 0.73 | 0.90 |
| Apx1 | S2PA | 0.89 | 1.81 | 1.12 | 2.03 |
| Apx1-1 | S2PA + 4A4J | 0.46 | 2.09 | 0.74 | 4.57 |
| Apx1-2 | S2PA + GCN4 | 0.54 | 2.15 | 0.82 | 3.97 |
| Apx2 | Cap1a | 0.50 | 1.38 | 0.72 | 2.76 |
| Apx2-1 | Cap1a + 4A4J | 0.24 | 1.32 | 0.51 | 5.39 |
| Apx2-2 | Cap1a + GCN4 | 0.16 | 0.97 | 0.36 | 6.19 |
| Apx2-3 | Cap1a + 4A4J + | 0.39 | 1.80 | 0.74 | 4.55 |
| | JSM-12 | | | | |
| Apx3 | Cap1b | 0.25 | 0.37 | 0.28 | 1.48 |
| Apx5 | Cap1xvw PPP | 0.53 | 1.29 | 0.72 | 2.46 |
| Apx5-1 | Cap1xvw PPP + | 0.30 | 1.40 | 0.58 | 4.59 |
| | 4A4J | | | | |
| Apx5-2 | Cap1xvw PPP + | 0.20 | 1.23 | 0.45 | 6.21 |
| | GCN4 | | | | |
| Apx5-3 | Cap1xvw PPP + | 0.20 | 0.83 | 0.36 | 4.07 |
| | 4A4J + JSM-12 | | | | |
| CL-25 | 436-GGP-489 | 0.69 | 1.31 | 0.81 | 1.88 |
| CL-26 | 436-GPP-489 | 0.06 | 0.16 | 0.07 | 2.62 |
| CL-27 | 436-PGP-489 | 0.04 | 0.06 | 0.04 | 1.49 |
| RMc | 436-GPG-489 | 0.03 | 0.06 | 0.04 | 1.91 |
| CL-2F-B | Apex w/furin | 1.14 | 5.10 | 1.69 | 4.50 |
| | cleavage sites | | | | |
| CL-2F- | Pf-70 + apex w/ | 1.43 | 3.64 | 1.74 | 2.54 |
| Pf-70 | furin cleavage sites | | | | |
| LZ-2 | ΔY189-Y201 | 1.83 | 3.66 | 2.20 | 2.00 |

Normalization was to the base construct (SEQ ID NO: 4)

TABLE 4
SEC Evaluation of Combination Designs

| | | | | Normalized | Peak 2 |
|---|---|---|---|---|---|
| | | Normalized | Normalized | Expression | AUC/ |
| Variant | | Peak 1 | Peak 2 | (Peak 1 + | Peak 1 |
| ID | Mutation(s) | AUC | AUC | Peak 2) | AUC |
| PB-22 | N220C/E657C | | | | |
| gB-53 | N220C/E657C/ | 1.18 | 0.98 | 1.06 | 0.83 |
| | V134C/I653C | | | | |
| gB-51 | N220C/E657C/ | 2.29 | 0.71 | 1.35 | 0.31 |
| | Y160C/Y708C | | | | |
| gB-47m | N220C/E657C/K260C/ | 0.47 | 0.20 | 0.31 | 0.41 |
| | S641C/D660Y | | | | |
| gB-49 | N220C/E657C/I356C/ | 1.77 | 0.61 | 1.08 | 0.34 |
| | A500C | | | | |
| gB-45 | N220C/E657C/ | 0.29 | 0.25 | 0.26 | 0.86 |
| | M371C/A505C | | | | |
| gB-43 | N220C/E657C/ | 0.27 | 0.07 | 0.15 | 0.27 |
| | Q612C/R662C | | | | |
| gB-11 | N220C/E657C/ | 1.27 | 1.08 | 1.15 | 0.85 |
| | S674C/E698C | | | | |
| gB-13 | N220C/E657C/ | 1.06 | 1.68 | 1.43 | 1.58 |
| | T100L/A267I | | | | |
| gB-65 | N220C/E657C/T100L/ | 0.94 | 0.77 | 0.84 | 0.81 |
| | A267I/Q98L/N658I | | | | |
| gB-23 | N220C/E657C/L102W | 0.79 | 0.88 | 0.85 | 1.11 |
| gB-27 | N220C/E657C/K130Y | 0.62 | 0.91 | 0.81 | 1.46 |
| gB-33 | N220C/E657C/L162W | 0.68 | 0.70 | 0.70 | 1.04 |
| gB-19 | N220C/E657C/E167I | 0.81 | 0.69 | 0.73 | 0.85 |
| gB-15 | N220C/E657C/K260W | 0.75 | 0.84 | 0.80 | 1.12 |
| gB-21 | N220C/E657C/V273F | 0.67 | 0.85 | 0.79 | 1.26 |
| gB-67 | N220C/E657C/L102W/ | 0.49 | 0.52 | 0.51 | 1.05 |
| | K130Y/L162W/ | | | | |
| | K260W/V273F | | | | |
| gB-17 | N220C/E657C/V645P | 1.10 | 1.19 | 1.15 | 1.08 |
| gB-64 | N220C/E657C/N341P/ | 0.93 | 1.66 | 1.36 | 1.80 |
| | L484P/V645P | | | | |
| gB-51-Pro | N220C/E657C/Y160C/ | 2.89 | 1.68 | 2.17 | 0.58 |
| | Y708C/N341P/ | | | | |
| | L484P/V645P | | | | |
| gB-31 | N220C/E657C/R693L/ | 0.84 | 0.69 | 0.74 | 0.82 |
| | K700V | | | | |
| PB-21 | V134C/I653C | | | | |
| gB-54 | V134C/I653C/K260C/ | 0.72 | 0.97 | 0.79 | 1.35 |
| | S641C | | | | |
| gB-57 | V134C/I653C/M371C/ | 0.90 | 1.00 | 0.95 | 1.11 |
| | A505C | | | | |
| gB-55 | V134C/I653C/Q612C/ | 0.14 | 0.05 | 0.11 | 0.36 |
| | R662C | | | | |
| gB-56 | V134C/I653C/S674C/ | 1.20 | 1.02 | 1.14 | 0.84 |
| | E698C | | | | |
| gB-75 | V134C/I653C/N566Y | 0.28 | 0.43 | 0.33 | 1.53 |
| gB-76 | V134C/I653C/E597F | 0.83 | 0.94 | 0.87 | 1.13 |
| gB-77m | V134C/I653C/E597Y/ | 0.05 | 0.07 | 0.06 | 1.30 |
| | I601T | | | | |
| gB-74 | V134C/I653C/N599Y | 0.21 | 0.19 | 0.20 | 0.88 |
| gB-72 | V134C/I653C/S643W | 0.76 | 0.75 | 0.76 | 0.99 |
| gB-73 | V134C/I653C/S647L | 0.39 | 0.32 | 0.37 | 0.81 |
| gB-62 | V134C/I653C/4J4A + Fd | 0.98 | 2.70 | 1.75 | 2.76 |
| MS-29 | H222C/E657C | | | | |
| gB-14 | H222C/E657C/K260W | 0.69 | 1.48 | 1.03 | 2.16 |
| gB-20 | H222C/E657C/V273F | 0.73 | 0.98 | 0.84 | 1.34 |
| gB-61 | H222C/E657C/4J4A + Fd | 0.40 | 1.20 | 0.74 | 3.00 |
| JSM-12 | T100L/A267I | | | | |
| gB-60 | T100L/A267I/4J4A + Fd | 0.64 | 1.38 | 1.03 | 2.16 |

Normalization was to the individual designs that head each section, which are bolded.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
Burke et al., “Crystal Structure of the Human Cytomegalovirus Glycoprotein B,” PLoS Pathog., 11:e1005227, 2015.
Chandramouli et al., “Structure of HCMV glycoprotein B in the postfusion conformation bound to a neutralizing human antibody,” Nat. Comm., 6:8176, 2015.
Liu et al., “Prefusion structure of human cytomegalovirus glycoprotein B and structural basis for membrane fusion,” Sci. Adv., 7:eabf3178, 2021.
Nelson et al., “Immune Correlates of Protection Against Human Cytomegalovirus Acquisition, Replication, and Disease,” J. Infect. Dis., 221:S45-S59, 2020.

1. (canceled)

2. An engineered human cytomegalovirus (HCMV) gB protein ectodomain comprising a sequence having at least 90% identity to amino acids 23-704 of SEQ ID NO: 1, said engineered protein comprising at least one substitution or set of substitutions selected from the group consisting of: Y89C/N599C; Y89C/G622C; V93C/V552C; V93C/L603C; A97C/R540C; A97C/A549C; G99C/L616C; R104C/I642C; R104C/T644C; E106C/T644C; L121C/N496C; M126C/W431C; V128C/V429C; V134C/I653C; A135C/I653C; Y153C/Y160C; Y153C/Y242C; Y160C/Y708C; N220C/E657C; T221C/T553C; H222C/E657C; K260C/S641C; V273C/L616C; T347C/A650C; I356C/A500C; M371C/A505C; Y415C/G433C; Y415C/K435C; F421C/V429C; G543C/K617C; G543C/F619C; D544C/Q597C; D544C/I620C; V545C/I618C; G604C/Y667C; Q612C/R662C; T644C/S647C; F661C/Y218C; S674C/E698C; D679C/R685C; D679C/E686C; E698C/R673C; N750C/G753C; L102W; I103Y; T112L; T112Y; V127F; K130W; K130Y; K130L; T159W; L162W; S223V/T253V/T270M/T659V; T253L; K260W; A267M; T270W; V273F; R354W; I356F; S367M; A396V; G433H; A500M; A500L/A503S; F541W; N556Y; E597F; E597Y; N599Y; R607Y; S641F; V645L; L616W; I620F; E627F; S643W; D646W; S647L; R662W; L664F; D679V/E682N; R685L/R607M; E686M; E686L/K691M; S689Y; G711L; L715F; L715F/V741Y/N750W; G731M; A738M; V741Y; I103R; V110E; D120R; Y153R/Y712E; Y155E; Y155E/G735K; Y155E/L709K; Y168E; Y218D/F584K; W240R; W240R/A732E; L241R/V728E; Y242D/L715K; V273K; I305E; R354E; E359R; R497E; Q501E; S526K; Y592E; N605E; H606E; L613D; P614R; D630R; M648E; A650D; I653D; D654E; L656D; V702E; K710E; V733D/F752K; K749E; N750K; E119P; 1121P; D122P; Y129P; A373P; A257P; K340P; N341P; A351P; G426P; L427P; H477P; V645P; A650P; L651P; E671P; L672P; I683P; M684P; E686P; K691P; G720P/A721P/A722P; A725P/V726P/A727P; Q98L/N658I; T100L/A267I; R104I; Y153F; L161E; L161R; L161W; E167I; F198W; T245E; T253L/T270L; D272N; A373F; D509L; D509R; R511Q; R512Q; R512W; S641E; T659V/N220F; E686N; R693L; R693L/K700V; and K700V, wherein the position are relative to SEQ ID NO: 1.

3. The engineered HCMV gB protein ectodomain of claim 2, comprising at least one set of paired cysteine substitutions selected from the group consisting of: Y89C/N599C; Y89C/G622C; V93C/V552C; V93C/L603C; A97C/R540C; A97C/A549C; G99C/L616C; R104C/I642C; R104C/T644C; E106C/T644C; L121C/N496C; M126C/W431C; V128C/V429C; V134C/I653C; A135C/I653C; Y153C/Y160C; Y153C/Y242C; Y160C/Y708C; N220C/E657C; T221C/T553C; H222C/E657C; K260C/S641C; V273C/L616C; T347C/A650C; I356C/A500C; M371C/A505C; Y415C/G433C; Y415C/K435C; F421C/V429C; G543C/K617C; G543C/F619C; D544C/Q597C; D544C/I620C; V545C/I618C; G604C/Y667C; Q612C/R662C; T644C/S647C; F661C/Y218C; S674C/E698C; D679C/R685C; D679C/E686C; E698C/R673C; E698C/S674C; and N750C/G753C.

4. The engineered HCMV gB protein ectodomain of claim 2, further comprising (1) at least one set of paired cysteine substitutions selected from the group consisting of: Q98C/N658C; L162C/M716C; S219C/N586C; S219C/A585C; W240C/G735C; Y242C/G731C G271C/P614C; W349C/A650C; S367C/A503C; L548C/P655C; A549C/N658C; S550C/P655C; S550C/E657C; and G604C/L672C; (2) at least one cavity filling substitution or set of cavity filling substitutions selected from the group consisting of: L102W; I103Y; T112L; T112Y; K130W; L162W; V127F; K130Y; K130L; T159W; S223V/T253V/T270M/T659V; T253L; K260W; A267M; T270W; V273F; R354W; I356F; S367M; A396V; G433H; A500M; A500L/A503S; F541W; N556Y; E597F; E597Y; N599Y; R607Y; S641F; V645L; L616W; I620F; E627F; S643W; D646W; S647L; R662W; L664F; D679V/E682N; R685L/R607M; E686M; E686L/K691M; S689Y; G711L; L715F; L715F/V741Y/N750W; G731M; A738M; and V741Y; (3) at least one substitution or set of substitutions selected from the group consisting of: I103R; V110E; D120R; Y153R/Y712E; Y155E; Y155E/G735K; Y155E/L709K; Y168E; Y218D/F584K; W240R; W240R/A732E; L241R/V728E; Y242D/L715K; V273K; I305E; R354E; E359R; R497E; 0501E; S526K; Y592E; N605E; H606E; L613D; P614R; D630R; M648E; A650D; I653D; D654E; L656D; V702E; K710E; V733D/F752K; K749E; and N750K; and/or (4) at least one substitution or set of substitutions selected from the group consisting of: E119P; L121P; D122P; Y129P; A373P; A257P; K340P; N341P; A351P; G426P; L427P; H477P; V645P; A650P; L651P; E671P; L672P; I683P; M684P; E686P; K691P; G720P/A721P/A722P; and A725P/V726P/A727P.

5-9. (canceled)

10. The engineered HCMV gB protein ectodomain of claim 2, further comprising at least one substitution selected from the group consisting of: N478P; L479P; V480P; Y481P; A482P; Q483P; L484P; and D646P.

11. The engineered HCMV gB protein ectodomain of claim 2, comprising or further comprising at least one substitution or set of substitutions selected from the group consisting of: Q98L/N658I; T100L/A267I; R104I; Y153F; L161E; L161R; L161W; E1671; F198W; T245E; T253L/T270L; D272N; A373F; D509L; D509R; R511Q; R512Q; R512W; S641E; T659V/N220F; E686N; R693L; R693L/K700V; and K700V.

12. The engineered HCMV gB protein ectodomain of claim 2, comprising or further comprising at least one substitution selected from the group consisting of: Y155G; I156H; Y157R; W240A; L241F; L241T; Y242H; C246S; R457S; R460S; D509A; and E686Q.

13. (canceled)

14. The engineered HCMV gB protein ectodomain of claim 2, wherein (1) positions 436-489 relative to SEQ ID NO: 1 have been replaced with GGP, GPP, PGP, or GPG; (2) positions 437-486 relative to SEQ ID NO: 1 have been replaced with CGADPQLC (SEQ ID NO: 76) or SGCDPQLC (SEQ ID NO: 77); or (3) positions 439-483 relative to SEQ ID NO: 1 have been replaced with RLNPP (SEQ ID NO: 78) or GADPQ (SEQ ID NO: 79), positions 439-484 relative to SEQ ID NO: 1 have been replaced with GMTPEQ (SEQ ID NO: 80), positions 437-486 relative to SEQ ID NO: 1 have been replaced with YDVCDPQLCY (SEQ ID NO: 81), or positions 439-487 relative to SEQ ID NO: 1 have been replaced with VGPPPTHKI (SEQ ID NO: 82).

15. (canceled)

16. The engineered HCMV gB protein ectodomain of claim 2, wherein positions 189-201 relative to SEQ ID NO: 1, positions 280-294 relative to SEQ ID NO: 1, or positions 265-284 relative to SEQ ID NO: 1 are deleted.

17-19. (canceled)

20. The engineered HCMV gB protein ectodomain of claim 2, comprising a set of substitutions selected from the group consisting of: V645/L102W/R693L/K700V; V645P/E686L/H222C/E657C; E106C/T644C/R104I/A267I/T100L/R693L/K700V; E106C/T644C/R104I/L102W/H222C/E657C; K130Y/A267I/T100L/R693L/K700V; K130Y/E686L/H222C/E657C; A503C/S367C/L102W/R693L/K700V; A503C/S367C/V273F/H222C/E657C; I356C/A500C/A267I/T100L/R693L/K700V; H222C/E657C/S674C/E698C; N220C/E657C/S674C/E698C; H222C/E657C/T100L/A267I; N220C/E657C/T100L/A267I; H222C/E657C/K260W; N220C/E657C/K260W; H222C/E657C/V645P; N220C/E657C/V645P; H222C/E657C/E167I; N220C/E657C/E167I; H222C/E657C/V273F; N220C/E657C/V273F; H222C/E657C/L102W; N220C/E657C/L102W; H222C/E657C/Q98L/N658I; N220C/E657C/Q98L/N658I; H222C/E657C/K130Y; N220C/E657C/K130Y; H222C/E657C/N341P; N220C/E657C/N341P; H222C/E657C/R693L/700V; N220C/E657C/R693L/K700V; H222C/E657C/L162W; N220C/E657C/L162W; H222C/E657C/V480P; N220C/E657C/V480P; H222C/E657C/L484P; N220C/E657C/L484P; H222C/E657C/L479P; N220C/E657C/L479P; H222C/E657C/Y481P; N220C/E657C/Y481P; H222C/E657C/Q612C/R662C; N220C/E657C/Q612C/R662C; H222C/E657C/M371C/A505C; N220C/E657C/M371C/A505C; H222C/E657C/K260C/S641C; N220C/E657C/K260C/S641C; H222C/E657C/I356C/A500C; N220C/E657C/I356C/A500C; H222C/E657C/Y160C/Y708C; N220C/E657C/Y160C/Y708C; H222C/E657C/V134C/I653C; N220C/E657C/V134C/I653C; V134C/I653C/K260C/S641C; V134C/I653C/Q612C/R662C; V134C/I653C/S674C/E698C; V134C/I653C/M371C/A505C; V134C/I653C/I356C/A500C; V134C/I653C/Y160C/Y708C; N220C/E657CN341P/L484P/V645P; N220C/E657C/T100L/A267I/Q98L/N658I; N220C/E657C/R693L/K700V/E686L/K691M; N220C/E657C/L102W/K130Y/L162W/K260W/V273F; V134C/I653C/N341P/L484P/V645P; V134C/I653C/T100L/A267I/Q98L/N658I; V134C/I653C/R693L/K700V/E686L/K691M; V134C/I653C/L102W/K130Y/L162W/K260W/V273F; V134C/I653C/S643W; V134C/I653C/S647L; V134C/I653C/N599Y; V134C/I653C/N566Y; V134C/I653C/E597F; V134C/I653C/E597Y; V134C/I653C/E627F; V134C/I653C/H606E; T100L/A267I/V134C/I653C/H222C/E657C; and V134C/I653C/H222C/E657C.

21. The engineered HCMV gB protein ectodomain of claim 2, wherein the set of substitutions comprises T100L, A267I, V134C, I653C, H222C, and E657C.

22. The engineered HCMV gB protein ectodomain of claim 2, which comprises a sequence having at least 91%, at least 92%, at least 93%, at least 94%, at least 95%, at least 96%, at least 97%, at least 98%, or at least 99% identity to (1) amino acids 23-704 of SEQ ID NO: 1; (2) amino acids 23-704 of SEQ ID NO: 4; or (3) the amino acid sequence of SEQ ID NO: 5.

23-24. (canceled)

25. An engineered protein comprising the engineered human cytomegalovirus (HCMV) gB protein ectodomain of claim 2.

26-27. (canceled)

28. The engineered protein of claim 25, wherein the engineered HCMV gB protein ectodomain is fused to a trimerization domain.

29. The engineered protein of claim 28, wherein the trimerization domain comprises a T4 fibritin trimerization domain, a GCN4 domain, a 4J4A domain, or a combination thereof.

30-34. (canceled)

35. The engineered protein of claim 25, wherein the engineered HCMV gB protein ectodomain is fused to a transmembrane domain.

36-38. (canceled)

39. The engineered protein of claim 25, which comprises a sequence having at least 91%, at least 92%, at least 93%, at least 94%, at least 95%, at least 96%, at least 97%, at least 98%, or at least 99% identity to amino acids 23-734 of SEQ ID NO: 4 or to the amino acid sequence of SEQ ID NO: 6.

40-42. (canceled)

43. An engineered human cytomegalovirus (HCMV) gB protein trimer comprising three engineered ectodomains or engineered proteins according to claim 2.

44-46. (canceled)

47. A nucleic acid molecule comprising a nucleotide sequence that encodes an amino acid sequence of the engineered ectodomain or engineered protein of claim 2.

48-52. (canceled)

53. A pharmaceutical composition comprising (i) the engineered ectodomain or engineered protein of claim 2; and a pharmaceutically acceptable carrier.

54-57. (canceled)

58. A method of (1) preventing human cytomegalovirus (HCMV) infection or a disease associated with HCMV infection, (2) eliciting an immune response, or (3) reducing cytomegalovirus viral shedding, in a subject, comprising administering to the subject an effective amount of the pharmaceutical composition of claim 53.

59-66. (canceled)


SEQUENCE LISTING (93 sequences)

SEQ ID NO: 1 - protein, 907 aa, Human cytomegalovirus
  source 1..907: note = native HCMV gB, strain Towne
  SIGNAL 1..22
MESRIWCLVVCVNLCIVCLGAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDDYSNTHSTRYVTVKDQWHSRGSTWLYRETCNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNRTKRSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLENTDFRVLELYSQKELRSSNVFDLEEIMREFNSYKQRVKYVEDKVVDPLPPYLKGLDDLMSGLGAAGKAVGVAIGAVGGAVASVVEGVATFLKNPFGAFTIILVAIAVVIIIYLIYTRQRRLCMQPLQNLFPYLVSADGTTVTSGNTKDTSLQAPPSYEESVYNSGRKGPGPPSSDASTAAPPYTNEQAYQMLLALVRLDAEQRAQQNGTDSLDGQTGTQDKGQKPNLLDRLRHRKNGYRHLKDSDEEENV

SEQ ID NO: 2 - protein, 907 aa, Human cytomegalovirus
  source 1..907: note = native HCMV gB, strain Merlin
MESRIWCLVVCVNLCIVCLGAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDDYSNTHSTRYVTVKDQWHSRGSTWLYRETCNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNRTKRSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLENTDFRVLELYSQKELRSSNVFDLEEIMREFNSYKQRVKYVEDKVVDPLPPYLKGLDDLMSGLGAAGKAVGVAIGAVGGAVASVVEGVATFLKNPFGAFTIILVAIAVVIITYLIYTRQRRLCTQPLQNLFPYLVSADGTTVTSGSTKDTSLQAPPSYEESVYNSGRKGPGPPSSDASTAAPPYTNEQAYQMLLALARLDAEQRAQQNGTDSLDGRTGTQDKGQKPNLLDRLRHRKNGYRHLKDSDEEENV

SEQ ID NO: 3 - protein, 906 aa, Human cytomegalovirus
  source 1..906: note = native HCMV gB, strain AD169
MESRIWCLVVCVNLCIVCLGAAVSSSSTSHATSSTHNGSHTSRTTSAQTRSVYSQHVTSSEAVSHRANETIYNTTLKYGDVVGVNTTKYPYRVCSMAQGTDLIRFERNIICTSMKPINEDLDEGIMVVYKRNIVAHTFKVRVYQKVLTFRRSYAYIYTTYLLGSNTEYVAPPMWEIHHINKFAQCYSSYSRVIGGTVFVAYHRDSYENKTMQLIPDDYSNTHSTRYVTVKDQWHSRGSTWLYRETCNLNCMLTITTARSKYPYHFFATSTGDVVYISPFYNGTNRNASYFGENADKFFIFPNYTIVSDFGRPNAAPETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETSGGLVVFWQGIKQKSLVELERLANRSSLNITHRTRRSTSDNNTTHLSSMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLENTDFRVLELYSQKELRSSNVFDLEEIMREFNSYKQRVKYVEDKVVDPLPPYLKGLDDLMSGLGAAGKAVGVAIGAVGGAVASVVEGVATFLKNPFGAFTIILVAIAVVIITYLIYTRQRRLCTQPLQNLFPYLVSADGTTVTSGSTKDTSLQAPPSYEESVYNSGRKGPGPPSSDASTAAPPYTNEQAYQMLLALARLDAEQRAQQNGTDSLDGQTGTQDKGQKPNLLDRLRHRKNGYRHLKDSDEEENV

SEQ ID NO: 4 - protein, 786 aa, synthetic construct
  source 1..786: note = Base Construct with signal sequence, Fd, and C-terminal tags
  SIGNAL 1..22
MESRIWCLVVCVNLCIVCLGAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDDYSNTHSTRYVTVKDQWHSRGSTWLYRETSNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLENTDFRVLELYSQKELRSSNVFDLEEIMREFNSYKQRVKYVEDKVVDPGSGYIPEAPRDGQAYVRKDGEWVLLSTFLGAAASLEVLFQGPGHHHHHHHHSAWSHPQFEKGGASGGGGSGGSAWSHPQFEK

SEQ ID NO: 5 - protein, 682 aa, synthetic construct
  source 1..682: note = Base Construct gB ectodomain only
VSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDDYSNTHSTRYVTVKDQWHSRGSTWLYRETSNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLENTDFRVLELYSQKELRSSNVFDLEEIMREFNSYKQRVKYVEDKVVDP

SEQ ID NO: 6 - protein, 712 aa, synthetic construct
  source 1..712: note = Base Construct gB ectodomain with Fd
VSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDDYSNTHSTRYVTVKDQWHSRGSTWLYRETSNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLENTDFRVLELYSQKELRSSNVFDLEEIMREFNSYKQRVKYVEDKVVDPGSGYIPEAPRDGQAYVRKDGEWVLLSTFLG

SEQ ID NO: 7 - protein, 786 aa, synthetic construct
  source 1..786: note = JSM-12 with signal sequence, Fd, and C-terminal tags
MESRIWCLVVCVNLCIVCLGAAVSSSSTRGTSATHSHHSSHTTSAAHSRSGSVSQRVTSSQTVSHGVNETIYNTTLKYGDVVGVNTTKYPYRVCSMAQGLDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDDYSNTHSTRYVTVKDQWHSRGSTWLYRETSNLNCMVTITTARSKYPYHFFITSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLENTDFRVLELYSQKELRSSNVFDLEEIMREFNSYKQRVKYVEDKVVDPGSGYIPEAPRDGQAYVRKDGEWVLLSTFLGAAASLEVLFQGPGHHHHHHHHSAWSHPQFEKGGASGGGGSGGSAWSHPQFEK

SEQ ID NO: 8 - protein, 682 aa, synthetic construct
  source 1..682: note = JSM-12 ectodomain only
VSSSSTRGTSATHSHHSSHTTSAAHSRSGSVSQRVTSSQTVSHGVNETIYNTTLKYGDVVGVNTTKYPYRVCSMAQGLDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDDYSNTHSTRYVTVKDQWHSRGSTWLYRETSNLNCMVTITTARSKYPYHFFITSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLENTDFRVLELYSQKELRSSNVFDLEEIMREFNSYKQRVKYVEDKVVDP

SEQ ID NO: 9 - protein, 712 aa, synthetic construct
  source 1..712: note = JSM-12 ectodomain with Fd
VSSSSTRGTSATHSHHSSHTTSAAHSRSGSVSQRVTSSQTVSHGVNETIYNTTLKYGDVVGVNTTKYPYRVCSMAQGLDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDDYSNTHSTRYVTVKDQWHSRGSTWLYRETSNLNCMVTITTARSKYPYHFFITSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLENTDFRVLELYSQKELRSSNVFDLEEIMREFNSYKQRVKYVEDKVVDPGSGYIPEAPRDGQAYVRKDGEWVLLSTFLG

SEQ ID NO: 10 - protein, 786 aa, synthetic construct
  source 1..786: note = PB-21 with signal sequence, Fd, and C-terminal tags
MESRIWCLVVCVNLCIVCLGAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNICAHTFKVRVYQKVLTFRRSYAYIHTTYLLGSNTEYVAPPMWEIHHINSHSQCYSSYSRVIAGTVFVAYHRDSYENKTMQLMPDDYSNTHSTRYVTVKDQWHSRGSTWLYRETSNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDCDPLENTDFRVLELYSQKELRSSNVFDLEEIMREFNSYKQRVKYVEDKVVDPGSGYIPEAPRDGQAYVRKDGEWVLLSTFLGAAASLEVLFQGPGHHHHHHHHSAWSHPQFEKGGASGGGGSGGSAWSHPQFEK

SEQ ID NO: 11 - protein, 682 aa, synthetic construct
  source 1..682: note = PB-21 ectodomain only
VSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNICAHTFKVRVYQKVLTFRRSYAYIHTTYLLGSNTEYVAPPMWEIHHINSHSQCYSSYSRVIAGTVFVAYHRDSYENKTMQLMPDDYSNTHSTRYVTVKDQWHSRGSTWLYRETSNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDCDPLENTDFRVLELYSQKELRSSNVFDLEEIMREFNSYKQRVKYVEDKVVDP

SEQ ID NO: 12 - protein, 712 aa, synthetic construct
  source 1..712: note = PB-21 ectodomain with Fd
VSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNICAHTFKVRVYQKVLTFRRSYAYIHTTYLLGSNTEYVAPPMWEIHHINSHSQCYSSYSRVIAGTVFVAYHRDSYENKTMQLMPDDYSNTHSTRYVTVKDQWHSRGSTWLYRETSNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDCDPLENTDFRVLELYSQKELRSSNVFDLEEIMREFNSYKQRVKYVEDKVVDPGSGYIPEAPRDGQAYVRKDGEWVLLSTFLG

SEQ ID NO: 13 - protein, 786 aa, synthetic construct
  source 1..786: note = MS-29 with signal sequence, Fd, and C-terminal tags
MESRIWCLVVCVNLCIVCLGAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDDYSNTCSTRYVTVKDQWHSRGSTWLYRETSNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLCNTDFRVLELYSQKELRSSNVFDLEEIMREFNSYKQRVKYVEDKVVDPGSGYIPEAPRDGQAYVRKDGEWVLLSTFLGAAASLEVLFQGPGHHHHHHHHSAWSHPQFEKGGASGGGGSGGSAWSHPQFEK

SEQ ID NO: 14 - protein, 682 aa, synthetic construct
  source 1..682: note = MS-29 ectodomain only
VSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDDYSNTCSTRYVTVKDQWHSRGSTWLYRETSNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLCNTDFRVLELYSQKELRSSNVFDLEEIMREFNSYKQRVKYVEDKVVDP

SEQ ID NO: 15 - protein, 712 aa, synthetic construct
  source 1..712: note = MS-29 ectodomain with Fd
VSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDDYSNTCSTRYVTVKDQWHSRGSTWLYRETSNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLCNTDFRVLELYSQKELRSSNVFDLEEIMREFNSYKQRVKYVEDKVVDPGSGYIPEAPRDGQAYVRKDGEWVLLSTFLG

SEQ ID NO: 16 - protein, 786 aa, synthetic construct
  source 1..786: note = JG-3 ED with signal sequence, Fd, and C-terminal tags
MESRIWCLVVCVNLCIVCLGAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYYRNIVAHTFKVRVYQKVLTFRRSYAYIHTTYLLGSNTEYVAPPMWEIHHINSHSQCYSSYSRVIAGTVFVAYHRDSYENKTMQLMPDDYSNTHSTRYVTVKDQWHSRGSTWLYRETSNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLENTDFRVLELYSQKELRSSNVFDLEEIMREFNSYKQRVKYVEDKVVDPGSGYIPEAPRDGQAYVRKDGEWVLLSTFLGAAASLEVLFQGPGHHHHHHHHSAWSHPQFEKGGASGGGGSGGSAWSHPQFEK

SEQ ID NO: 17 - protein, 682 aa, synthetic construct
  source 1..682: note = JG-3 ectodomain only
VSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYYRNIVAHTFKVRVYQKVLTFRRSYAYIHTTYLLGSNTEYVAPPMWEIHHINSHSQCYSSYSRVIAGTVFVAYHRDSYENKTMQLMPDDYSNTHSTRYVTVKDQWHSRGSTWLYRETSNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLENTDFRVLELYSQKELRSSNVFDLEEIMREFNSYKQRVKYVEDKVVDP

SEQ ID NO: 18 - protein, 712 aa, synthetic construct
  source 1..712: note = JG-3 ectodomain with Fd
VSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYYRNIVAHTFKVRVYQKVLTFRRSYAYIHTTYLLGSNTEYVAPPMWEIHHINSHSQCYSSYSRVIAGTVFVAYHRDSYENKTMQLMPDDYSNTHSTRYVTVKDQWHSRGSTWLYRETSNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLENTDFRVLELYSQKELRSSNVFDLEEIMREFNSYKQRVKYVEDKVVDPGSGYIPEAPRDGQAYVRKDGEWVLLSTFLG

SEQ ID NO: 19 - protein, 786 aa, synthetic construct
  source 1..786: note = JSM-21 with signal sequence, Fd, and C-terminal tags
MESRIWCLVVCVNLCIVCLGAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDDYSNTHSTRYVTVKDQWHSRGSTWLYRETSNLNCMVTITTARSKYPYHFFATSTGDFVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLENTDFRVLELYSQKELRSSNVFDLEEIMREFNSYKQRVKYVEDKVVDPGSGYIPEAPRDGQAYVRKDGEWVLLSTFLGAAASLEVLFQGPGHHHHHHHHSAWSHPQFEKGGASGGGGSGGSAWSHPQFEK

SEQ ID NO: 20 - protein, 682 aa, synthetic construct
  source 1..682: note = JSM-21 ectodomain only
VSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDDYSNTHSTRYVTVKDQWHSRGSTWLYRETSNLNCMVTITTARSKYPYHFFATSTGDFVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLENTDFRVLELYSQKELRSSNVFDLEEIMREFNSYKQRVKYVEDKVVDP

SEQ ID NO: 21 - protein, 712 aa, synthetic construct
  source 1..712: note = JSM-21 ectodomain with Fd
VSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDDYSNTHSTRYVTVKDQWHSRGSTWLYRETSNLNCMVTITTARSKYPYHFFATSTGDFVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLENTDFRVLELYSQKELRSSNVFDLEEIMREFNSYKQRVKYVEDKVVDPGSGYIPEAPRDGQAYVRKDGEWVLLSTFLG

SEQ ID NO: 22 - protein, 786 aa, synthetic construct
  source 1..786: note = JG-8 with signal sequence, Fd, and C-terminal tags
MESRIWCLVVCVNLCIVCLGAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDDYSNTHSTRYVTVKDQWHSRGSTWLYRETSNLNCMVTITTARSWYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLENTDFRVLELYSQKELRSSNVFDLEEIMREFNSYKQRVKYVEDKVVDPGSGYIPEAPRDGQAYVRKDGEWVLLSTFLGAAASLEVLFQGPGHHHHHHHHSAWSHPQFEKGGASGGGGSGGSAWSHPQFEK

SEQ ID NO: 23 - protein, 682 aa, synthetic construct
  source 1..682: note = JG-8 ectodomain only
VSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDDYSNTHSTRYVTVKDQWHSRGSTWLYRETSNLNCMVTITTARSWYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLENTDFRVLELYSQKELRSSNVFDLEEIMREFNSYKQRVKYVEDKVVDP

SEQ ID NO: 24 - protein, 712 aa, synthetic construct
  source 1..712: note = JG-8 ectodomain with Fd
VSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDDYSNTHSTRYVTVKDQWHSRGSTWLYRETSNLNCMVTITTARSWYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLENTDFRVLELYSQKELRSSNVFDLEEIMREFNSYKQRVKYVEDKVVDPGSGYIPEAPRDGQAYVRKDGEWVLLSTFLG

SEQ ID NO: 25 - protein, 786 aa, synthetic construct
  source 1..786: note = PB-22 with signal sequence, Fd, and C-terminal tags
MESRIWCLVVCVNLCIVCLGAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDDYSCTHSTRYVTVKDQWHSRGSTWLYRETSNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLCNTDFRVLELYSQKELRSSNVFDLEEIMREFNSYKQRVKYVEDKVVDPGSGYIPEAPRDGQAYVRKDGEWVLLSTFLGAAASLEVLFQGPGHHHHHHHHSAWSHPQFEKGGASGGGGSGGSAWSHPQFEK

SEQ ID NO: 26 - protein, 682 aa, synthetic construct
  source 1..682: note = PB-22 ectodomain only
VSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDDYSCTHSTRYVTVKDQWHSRGSTWLYRETSNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLCNTDFRVLELYSQKELRSSNVFDLEEIMREFNSYKQRVKYVEDKVVDP

SEQ ID NO: 27 - protein, 712 aa, synthetic construct
  source 1..712: note = PB-22 ectodomain with Fd
VSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDDYSCTHSTRYVTVKDQWHSRGSTWLYRETSNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLCNTDFRVLELYSQKELRSSNVFDLEEIMREFNSYKQRVKYVEDKVVDPGSGYIPEAPRDGQAYVRKDGEWVLLSTFLG

SEQ ID NO: 28 - protein, 786 aa, synthetic construct
  source 1..786: note = gB-12 with signal sequence, Fd, and C-terminal tags
MESRIWCLVVCVNLCIVCLGAAVSSSSTRGTSATHSHHSSHTTSAAHSRSGSVSQRVTSSQTVSHGVNETIYNTTLKYGDVVGVNTTKYPYRVCSMAQGLDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDDYSNTCSTRYVTVKDQWHSRGSTWLYRETSNLNCMVTITTARSKYPYHFFITSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLCNTDFRVLELYSQKELRSSNVFDLEEIMREFNSYKQRVKYVEDKVVDPGSGYIPEAPRDGQAYVRKDGEWVLLSTFLGAAASLEVLFQGPGHHHHHHHHSAWSHPQFEKGGASGGGGSGGSAWSHPQFEK

SEQ ID NO: 29 - protein, 682 aa, synthetic construct
  source 1..682: note = gB-12 ectodomain
VSSSSTRGTSATHSHHSSHTTSAAHSRSGSVSQRVTSSQTVSHGVNETIYNTTLKYGDVVGVNTTKYPYRVCSMAQGLDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDDYSNTCSTRYVTVKDQWHSRGSTWLYRETSNLNCMVTITTARSKYPYHFFITSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLCNTDFRVLELYSQKELRSSNVFDLEEIMREFNSYKQRVKYVEDKVVDP

SEQ ID NO: 30 - protein, 712 aa, synthetic construct
  source 1..712: note = gB-12 ectodomain with Fd
VSSSSTRGTSATHSHHSSHTTSAAHSRSGSVSQRVTSSQTVSHGVNETIYNTTLKYGDVVGVNTTKYPYRVCSMAQGLDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDDYSNTCSTRYVTVKDQWHSRGSTWLYRETSNLNCMVTITTARSKYPYHFFITSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLCNTDFRVLELYSQKELRSSNVFDLEEIMREFNSYKQRVKYVEDKVVDPGSGYIPEAPRDGQAYVRKDGEWVLLSTFLG

SEQ ID NO: 31 - protein, 786 aa, synthetic construct
  source 1..786: note = gB-13 with signal sequence, Fd, and C-terminal tags
MESRIWCLVVCVNLCIVCLGAAVSSSSTRGTSATHSHHSSHTTSAAHSRSGSVSQRVTSSQTVSHGVNETIYNTTLKYGDVVGVNTTKYPYRVCSMAQGLDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDDYSCTHSTRYVTVKDQWHSRGSTWLYRETSNLNCMVTITTARSKYPYHFFITSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLCNTDFRVLELYSQKELRSSNVFDLEEIMREFNSYKQRVKYVEDKVVDPGSGYIPEAPRDGQAYVRKDGEWVLLSTFLGAAASLEVLFQGPGHHHHHHHHSAWSHPQFEKGGASGGGGSGGSAWSHPQFEK

SEQ ID NO: 32 - protein, 682 aa, synthetic construct
  source 1..682: note = gB-13 ectodomain
VSSSSTRGTSATHSHHSSHTTSAAHSRSGSVSQRVTSSQTVSHGVNETIYNTTLKYGDVVGVNTTKYPYRVCSMAQGLDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDDYSCTHSTRYVTVKDQWHSRGSTWLYRETSNLNCMVTITTARSKYPYHFFITSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLCNTDFRVLELYSQKELRSSNVFDLEEIMREFNSYKQRVKYVEDKVVDP

SEQ ID NO: 33 - protein, 712 aa, synthetic construct
  source 1..712: note = gB-13 ectodomain with Fd
VSSSSTRGTSATHSHHSSHTTSAAHSRSGSVSQRVTSSQTVSHGVNETIYNTTLKYGDVVGVNTTKYPYRVCSMAQGLDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDDYSCTHSTRYVTVKDQWHSRGSTWLYRETSNLNCMVTITTARSKYPYHFFITSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLCNTDFRVLELYSQKELRSSNVFDLEEIMREFNSYKQRVKYVEDKVVDPGSGYIPEAPRDGQAYVRKDGEWVLLSTFLG

SEQ ID NO: 34 - protein, 786 aa, synthetic construct
  source 1..786: note = gB-14 with signal sequence, Fd, and C-terminal tags
MESRIWCLVVCVNLCIVCLGAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDDYSNTCSTRYVTVKDQWHSRGSTWLYRETSNLNCMVTITTARSWYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLCNTDFRVLELYSQKELRSSNVFDLEEIMREFNSYKQRVKYVEDKVVDPGSGYIPEAPRDGQAYVRKDGEWVLLSTFLGAAASLEVLFQGPGHHHHHHHHSAWSHPQFEKGGASGGGGSGGSAWSHPQFEK

SEQ ID NO: 35 - protein, 682 aa, synthetic construct
  source 1..682: note = gB-14 ectodomain only
VSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDDYSNTCSTRYVTVKDQWHSRGSTWLYRETSNLNCMVTITTARSWYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLCNTDFRVLELYSQKELRSSNVFDLEEIMREFNSYKQRVKYVEDKVVDP

SEQ ID NO: 36 - protein, 712 aa, synthetic construct
  source 1..712: note = gB-14 ectodomain with Fd
VSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDDYSNTCSTRYVTVKDQWHSRGSTWLYRETSNLNCMVTITTARSWYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLCNTDFRVLELYSQKELRSSNVFDLEEIMREFNSYKQRVKYVEDKVVDPGSGYIPEAPRDGQAYVRKDGEWVLLSTFLG

SEQ ID NO: 37 - protein, 786 aa, synthetic construct
  source 1..786: note = gB-20 with signal sequence, Fd, and C-terminal tags
MESRIWCLVVCVNLCIVCLGAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDDYSNTCSTRYVTVKDQWHSRGSTWLYRETSNLNCMVTITTARSKYPYHFFATSTGDFVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLCNTDFRVLELYSQKELRSSNVFDLEEIMREFNSYKQRVKYVEDKVVDPGSGYIPEAPRDGQAYVRKDGEWVLLSTFLGAAASLEVLFQGPGHHHHHHHHSAWSHPQFEKGGASGGGGSGGSAWSHPQFEK

SEQ ID NO: 38 - protein, 682 aa, synthetic construct
  source 1..682: note = gB-20 ectodomain
VSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDDYSNTCSTRYVTVKDQWHSRGSTWLYRETSNLNCMVTITTARSKYPYHFFATSTGDFVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLCNTDFRVLELYSQKELRSSNVFDLEEIMREFNSYKQRVKYVEDKVVDP

SEQ ID NO: 39 - protein, 712 aa, synthetic construct
  source 1..712: note = gB-20 ectodomain with Fd
VSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDDYSNTCSTRYVTVKDQWHSRGSTWLYRETSNLNCMVTITTARSKYPYHFFATSTGDFVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLCNTDFRVLELYSQKELRSSNVFDLEEIMREFNSYKQRVKYVEDKVVDPGSGYIPEAPRDGQAYVRKDGEWVLLSTFLG

SEQ ID NO: 40 - protein, 786 aa, synthetic construct
  source 1..786: note = gB-27 with signal sequence, Fd, and C-terminal tags
MESRIWCLVVCVNLCIVCLGAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYYRNIVAHTFKVRVYQKVLTFRRSYAYIHTTYLLGSNTEYVAPPMWEIHHINSHSQCYSSYSRVIAGTVFVAYHRDSYENKTMQLMPDDYSCTHSTRYVTVKDQWHSRGSTWLYRETSNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLCNTDFRVLELYSQKELRSSNVFDLEEIMREFNSYKQRVKYVEDKVVDPGSGYIPEAPRDGQAYVRKDGEWVLLSTFLGAAASLEVLFQGPGHHHHHHHHSAWSHPQFEKGGASGGGGSGGSAWSHPQFEK

SEQ ID NO: 41 - protein, 682 aa, synthetic construct
  source 1..682: note = gB-27 ectodomain only
VSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYYRNIVAHTFKVRVYQKVLTFRRSYAYIHTTYLLGSNTEYVAPPMWEIHHINSHSQCYSSYSRVIAGTVFVAYHRDSYENKTMQLMPDDYSCTHSTRYVTVKDQWHSRGSTWLYRETSNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLCNTDFRVLELYSQKELRSSNVFDLEEIMREFNSYKQRVKYVEDKVVDP

SEQ ID NO: 42 - protein, 712 aa, synthetic construct
  source 1..712: note = gB-27 ectodomain with Fd
VSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYYRNIVAHTFKVRVYQKVLTFRRSYAYIHTTYLLGSNTEYVAPPMWEIHHINSHSQCYSSYSRVIAGTVFVAYHRDSYENKTMQLMPDDYSCTHSTRYVTVKDQWHSRGSTWLYRETSNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLCNTDFRVLELYSQKELRSSNVFDLEEIMREFNSYKQRVKYVEDKVVDPGSGYIPEAPRDGQAYVRKDGEWVLLSTFLG

SEQ ID NO: 43 - protein, 786 aa, synthetic construct
  source 1..786: note = gB-53 with signal sequence, Fd, and C-terminal tags
MESRIWCLVVCVNLCIVCLGAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNICAHTFKVRVYQKVLTFRRSYAYIHTTYLLGSNTEYVAPPMWEIHHINSHSQCYSSYSRVIAGTVFVAYHRDSYENKTMQLMPDDYSCTHSTRYVTVKDQWHSRGSTWLYRETSNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDCDPLCNTDFRVLELYSQKELRSSNVFDLEEIMREFNSYKQRVKYVEDKVVDPGSGYIPEAPRDGQAYVRKDGEWVLLSTFLGAAASLEVLFQGPGHHHHHHHHSAWSHPQFEKGGASGGGGSGGSAWSHPQFEK

SEQ ID NO: 44 - protein, 682 aa, synthetic construct
  source 1..682: note = gB-53 ectodomain only
VSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNICAHTFKVRVYQKVLTFRRSYAYIHTTYLLGSNTEYVAPPMWEIHHINSHSQCYSSYSRVIAGTVFVAYHRDSYENKTMQLMPDDYSCTHSTRYVTVKDQWHSRGSTWLYRETSNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDCDPLCNTDFRVLELYSQKELRSSNVFDLEEIMREFNSYKQRVKYVEDKVVDP

SEQ ID NO: 45 - protein, 712 aa, synthetic construct
  source 1..712: note = gB-53 ectodomain with Fd
VSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNICAHTFKVRVYQKVLTFRRSYAYIHTTYLLGSNTEYVAPPMWEIHHINSHSQCYSSYSRVIAGTVFVAYHRDSYENKTMQLMPDDYSCTHSTRYVTVKDQWHSRGSTWLYRETSNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDCDPLCNTDFRVLELYSQKELRSSNVFDLEEIMREFNSYKQRVKYVEDKVVDPGSGYIPEAPRDGQAYVRKDGEWVLLSTFLG

SEQ ID NO: 46 - protein, 755 aa, synthetic construct
  source 1..755: note = gB-62 with signal sequence and C-terminal tags
MESRIWCLVVCVNLCIVCLGAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNICAHTFKVRVYQKVLTFRRSYAYIHTTYLLGSNTEYVAPPMWEIHHINSHSQCYSSYSRVIAGTVFVAYHRDSYENKTMQLMPDDYSNTHSTRYVTVKDQWHSRGSTWLYRETSNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDCDPLENTDFRVLELYSQKELRSSNVFDLEEIMREFNSLKQIVLRIMEIEARIAKIEGSGYIPEAPRDGQAYVRKDGEWVLLSTFLGSHHHHHHSAWSHPQFEK

SEQ ID NO: 47 - protein, 716 aa, synthetic construct
  source 1..716: note = gB-62 ectodomain with 4J4A+Fd
VSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNICAHTFKVRVYQKVLTFRRSYAYIHTTYLLGSNTEYVAPPMWEIHHINSHSQCYSSYSRVIAGTVFVAYHRDSYENKTMQLMPDDYSNTHSTRYVTVKDQWHSRGSTWLYRETSNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDCDPLENTDFRVLELYSQKELRSSNVFDLEEIMREFNSLKQIVLRIMEIEARIAKIEGSGYIPEAPRDGQAYVRKDGEWVLLSTFLG

SEQ ID NO: 48 - protein, 755 aa, synthetic construct
  source 1..755: note = gB-63 with signal sequence and C-terminal tags
MESRIWCLVVCVNLCIVCLGAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDDYSCTHSTRYVTVKDQWHSRGSTWLYRETSNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLCNTDFRVLELYSQKELRSSNVFDLEEIMREFNSLKQIVLRIMEIEARIAKIEGSGYIPEAPRDGQAYVRKDGEWVLLSTFLGSHHHHHHSAWSHPQFEK

SEQ ID NO: 49 - protein, 716 aa, synthetic construct
  source 1..716: note = gB-63 ectodomain with 4J4A+Fd
VSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDDYSCTHSTRYVTVKDQWHSRGSTWLYRETSNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLCNTDFRVLELYSQKELRSSNVFDLEEIMREFNSLKQIVLRIMEIEARIAKIEGSGYIPEAPRDGQAYVRKDGEWVLLSTFLG

SEQ ID NO: 50 - protein, 786 aa, synthetic construct
  source 1..786: note = gB100 with signal sequence, Fd, and C-terminal tags
MESRIWCLVVCVNLCIVCLGAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNICAHTFKVRVYQKVLTFRRSYAYIHTTYLLGSNTEYVAPPMWEIHHINSHSQCYSSYSRVIAGTVFVAYHRDSYENKTMQLMPDDYSNTCSTRYVTVKDQWHSRGSTWLYRETSNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDCDPLCNTDFRVLELYSQKELRSSNVFDLEEIMREFNSYKQRVKYVEDKVVDPGSGYIPEAPRDGQAYVRKDGEWVLLSTFLGAAASLEVLFQGPGHHHHHHHHSAWSHPQFEKGGASGGGGSGGSAWSHPQFEK

SEQ ID NO: 51 - protein, 682 aa, synthetic construct
  source 1..682: note = gB-100 ectodomain only
VSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNICAHTFKVRVYQKVLTFRRSYAYIHTTYLLGSNTEYVAPPMWEIHHINSHSQCYSSYSRVIAGTVFVAYHRDSYENKTMQLMPDDYSNTCSTRYVTVKDQWHSRGSTWLYRETSNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDCDPLCNTDFRVLELYSQKELRSSNVFDLEEIMREFNSYKQRVKYVEDKVVDP

SEQ ID NO: 52 - protein, 712 aa, synthetic construct
  source 1..712: note = gB-100 ectodomain with Fd
VSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNICAHTFKVRVYQKVLTFRRSYAYIHTTYLLGSNTEYVAPPMWEIHHINSHSQCYSSYSRVIAGTVFVAYHRDSYENKTMQLMPDDYSNTCSTRYVTVKDQWHSRGSTWLYRETSNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDCDPLCNTDFRVLELYSQKELRSSNVFDLEEIMREFNSYKQRVKYVEDKVVDPGSGYIPEAPRDGQAYVRKDGEWVLLSTFLG

SEQ ID NO: 53 - protein, 786 aa, synthetic construct
  source 1..786: note = gB107 with signal sequence, Fd, and C-terminal tags
MESRIWCLVVCVNLCIVCLGAAVSSSSTRGTSATHSHHSSHTTSAAHSRSGSVSQRVTSSQTVSHGVNETIYNTTLKYGDVVGVNTTKYPYRVCSMAQGLDLIRFERNIVCTSMKPINEDLDEGIMVVYKRNICAHTFKVRVYQKVLTFRRSYAYIHTTYLLGSNTEYVAPPMWEIHHINSHSQCYSSYSRVIAGTVFVAYHRDSYENKTMQLMPDDYSNTCSTRYVTVKDQWHSRGSTWLYRETSNLNCMVTITTARSKYPYHFFITSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDCDPLCNTDFRVLELYSQKELRSSNVFDLEEIMREFNSYKQRVKYVEDKVVDPGSGYIPEAPRDGQAYVRKDGEWVLLSTFLGAAASLEVLFQGPGHHHHHHHHSAWSHPQFEKGGASGGGGSGGSAWSHPQFEK

SEQ ID NO: 54 - protein, 682 aa, synthetic construct
  source 1..682: note = gB-107 ectodomain only
VSSSSTRGTSATHSHHSSHTTSAAHSRSGSVSQRVTSSQTVSHGVNETIYNTTLKYGDVVGVNTTKYPYRVCSMAQGLDLIRFERNIVCTSMKPINEDLDEGIMVVYKRNICAHTFKVRVYQKVLTFRRSYAYIHTTYLLGSNTEYVAPPMWEIHHINSHSQCYSSYSRVIAGTVFVAYHRDSYENKTMQLMPDDYSNTCSTRYVTVKDQWHSRGSTWLYRETSNLNCMVTITTARSKYPYHFFITSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDCDPLCNTDFRVLELYSQKELRSSNVFDLEEIMREFNSYKQRVKYVEDKVVDP

SEQ ID NO: 55 - protein, 712 aa, synthetic construct
  source 1..712: note = gB-107 ectodomain with Fd
VSSSSTRGTSATHSHHSSHTTSAAHSRSGSVSQRVTSSQTVSHGVNETIYNTTLKYGDVVGVNTTKYPYRVCSMAQGLDLIRFERNIVCTSMKPINEDLDEGIMVVYKRNICAHTFKVRVYQKVLTFRRSYAYIHTTYLLGSNTEYVAPPMWEIHHINSHSQCYSSYSRVIAGTVFVAYHRDSYENKTMQLMPDDYSNTCSTRYVTVKDQWHSRGSTWLYRETSNLNCMVTITTARSKYPYHFFITSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDCDPLCNTDFRVLELYSQKELRSSNVFDLEEIMREFNSYKQRVKYVEDKVVDPGSGYIPEAPRDGQAYVRKDGEWVLLSTFLG

SEQ ID NO: 56 - protein, 746 aa, synthetic construct
  source 1..746: note = Apx-1
MESRIWCLVVCVNLCIVCLGAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDDYSNTHSTRYVTVKDQWHSRGSTWLYRETSNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSRLNPPLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLENTDFRVLELYSQKELRSSNVFDLEEIMREFNSYKQRVKYVEDKVVDPGSGYIPEAPRDGQAYVRKDGEWVLLSTFLGAAASLEVLFQGPGHHHHHHHHSAWSHPQFEKGGASGGGGSGGSAWSHPQFEK

SEQ ID NO: 57 - protein, 746 aa, synthetic construct
  source 1..746: note = Apx-2
MESRIWCLVVCVNLCIVCLGAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDDYSNTHSTRYVTVKDQWHSRGSTWLYRETSNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSGADPQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLENTDFRVLELYSQKELRSSNVFDLEEIMREFNSYKQRVKYVEDKVVDPGSGYIPEAPRDGQAYVRKDGEWVLLSTFLGAAASLEVLFQGPGHHHHHHHHSAWSHPQFEKGGASGGGGSGGSAWSHPQFEK

SEQ ID NO: 58 - protein, 746 aa, synthetic construct
  source 1..746: note = Apx-3
MESRIWCLVVCVNLCIVCLGAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDDYSNTHSTRYVTVKDQWHSRGSTWLYRETSNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSGMTPEQQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLENTDFRVLELYSQKELRSSNVFDLEEIMREFNSYKQRVKYVEDKVVDPGSGYIPEAPRDGQAYVRKDGEWVLLSTFLGAAASLEVLFQGPGHHHHHHHHSAWSHPQFEKGGASGGGGSGGSAWSHPQFEK

SEQ ID NO: 59 - protein, 746 aa, synthetic construct
  source 1..746: note = Apx-4
MESRIWCLVVCVNLCIVCLGAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDDYSNTHSTRYVTVKDQWHSRGSTWLYRETSNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQYDVCDPQLCYTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLENTDFRVLELYSQKELRSSNVFDLEEIMREFNSYKQRVKYVEDKVVDPGSGYIPEAPRDGQAYVRKDGEWVLLSTFLGAAASLEVLFQGPGHHHHHHHHSAWSHPQFEKGGASGGGGSGGSAWSHPQFEK

SEQ ID NO: 60 - protein, 746 aa, synthetic construct
  source 1..746: note = Apx-5
MESRIWCLVVCVNLCIVCLGAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDDYSNTHSTRYVTVKDQWHSRGSTWLYRETSNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSVGPPPTHKIYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLENTDFRVLELYSQKELRSSNVFDLEEIMREFNSYKQRVKYVEDKVVDPGSGYIPEAPRDGQAYVRKDGEWVLLSTFLGAAASLEVLFQGPGHHHHHHHHSAWSHPQFEKGGASGGGGSGGSAWSHPQFEK

SEQ ID NO: 61 - protein, 739 aa, synthetic construct
  source 1..739: note = JSM-GCN4
MESRIWCLVVCVNLCIVCLGAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDDYSNTHSTRYVTVKDQWHSRGSTWLYRETCNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLENTDFRVLELYSQKELRSSNVFDLEEIMREFNSYKQRDTYLSAIEDKIEEILSKIYHIENEIARIGSHHHHHHSAWSHPQFEK

SEQ ID NO: 62 - protein, 726 aa, synthetic construct
  source 1..726: note = JSM-4J4A
MESRIWCLVVCVNLCIVCLGAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDDYSNTHSTRYVTVKDQWHSRGSTWLYRETCNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLENTDFRVLELYSQKELRSSNVFDLEEIMREFNSLKQIVLRIMEIEARIAKIEGSHHHHHHSAWSHPQFEK

SEQ ID NO: 63 - protein, 742 aa, Human cytomegalovirus
MRPGLPSYLIILAVCLFSHLLSSRYGAEAVSEPLDKAFHLLLNTYGRPIRFLRENTTQCTYNSSLRNSTVVRENAISFNFFQSYNQYYVFHMPRCLFAGPLAEQFLNQVDLTETLERYQQRLNTYALVSKDLASYRSFSQQLKAQDSLGEQPTTVPPPIDLSIPHVWMPPQTTPHGWTESHTTSGLHRPHFNQTCILFDGHDLLFSTVTPCLHQGFYLIDELRYVKITLTEDFFVVTVSIDDDTPMLLIFGHLPRVLFKAPYQRDNFILRQTEKHELLVLVKKDQLNRHSYLKDPDFLDAALDFNYLDLSALLRNSFHRYAVDVLKSGRCQMLDRRTVEMAFAYALALFAAARQEEAGAQVSVPRALDRQAALLQIQEFMITCLSQTPPRTTLLLYPTAVDLAKRALWTPNQITDITSLVRLVYILSKQNQQHLIPQWALRQIADFALKLHKTHLASFLSAFARQELYLMGSLVHSMLVHTTERREIFIVETGLCSLAELSHFTQLLAHPHHEYLSDLYTPCSSSGRRDHSLERLTRLFPDATVPATVPAALSILSTMQPSTLETFPDLFCLPLGESFSALTVSEHVSYIVTNQYLIKGISYPVSTTVVGQSLIITQTDSQTKCELTRNMHTTHSITVALNISLENCAFCQSALLEYDDTQGVINIMYMHDSDDVLFALDPYNEVVVSSPRTHYLMLLKNGTVLEVTDVVVDATDSRLLMMSVYALSAIIGIYLLYRMLKTC

SEQ ID NO: 64 - protein, 278 aa, Human cytomegalovirus
MCRRPDCGFSFSPGPVILLWCCLLLPIVSSAAVSVAPTAAEKVPAECPELTRRCLLGEVFEGDKYESWLRPLVNVTGRDGPLSQLIRYRPVTPEAANSVLLDEAFLDTLALLYNNPDQLRALLTLLSSDTAPRWMTVMRGYSECGDGSPAVYTCVDDLCRGYDLTRLSYGRSIFTEHVLGFELVPPSLFNVVVAIRNEATRTNRAVRLPVSTAAAPEGITLFYGLYNAVKEFCLRHQLDPPLLRHLDKYYAGLPPELKQTRVNLPAHSRYGPQAVDAR

SEQ ID NO: 65 - protein, 171 aa, Human cytomegalovirus
MSPKDLTPFLTALWLLLGHSRVPRVRAEECCEFINVNHPPERCYDFKMCNRFTVALRCPDGEVCYSPEKTAEIRGIVTTMTHSLTRQVVHNKLTSCNYNPLYLEADGRIRCGKVNDKAQYLLGAAGSVPYRWINLEYDKITRIVGLDQYLESVKKHKRLDVCRAKMGYMLQ

SEQ ID NO: 66 - protein, 214 aa, Human cytomegalovirus
MLRLLLRHHFHCLLLCAVWATPCLASPWSTLTANQNPSPPWSKLTYSKPHDAATFYCPFLYPSPPRSPLQFSGFQRVSTGPECRNETLYLLYNREGQTLVERSSTWVKKVIWYLSGRNQTILQRMPRTASKPSDGNVQISVEDAKIFGAHMVPKQTKLLRFVVNDGTRYQMCVMKLESWAHVFRDYSVSFQVRLTFTEANNQTYTFCTHPNLIV

SEQ ID NO: 67 - protein, 129 aa, Human cytomegalovirus
MRLCRVWLSVCLCAVVLGQCQRETAEKNDYYRVPHYWDACSRALPDQTRYKYVEQLVDLTLNYHYDASHGLDNFDVLKRINVTEVSLLISDFRRQNRRGGTNKRTTFNAAGSLAPHARSLEFSVRLFAN

SEQ ID NO: 68 - protein, 907 aa, Human cytomegalovirus; identical to SEQ ID NO: 2

SEQ ID NO: 69 - protein, 557 aa, Human cytomegalovirus
MESRGRRCPEMISVLGPISGHVLKAVFSRGDTPVLPHETRLLQTGIHVRVSQPSLILVSQYTPDSTPCHRGDNQLQVQHTYFTGSEVENVSVNVHNPTGRSICPSQEPMSIYVYALPLKMLNIPSINVHHYPSAAERKHRHLPVADAVIHASGKQMWQARLTVSGLAWTRQQNQWKEPDVYYTSAFVFPTKDVALRHVVCAHELVCSMENTRATKMQVIGDQYVKVYLESFCEDVPSGKLFMHVTLGSDVEEDLTMTRNPQPFMRPHERNGFTVLCPKNMIIKPGKISHIMLDVAFTSHEHFGLLCPKSIPGLSISGNLLMNGQQIFLEVQAIRETVELRQYDPVAALFFFDIDLLLQRGPQYSEHPTFTSQYRIQGKLEYRHTWDRHDEGAAQGDDDVWTSGSDSDEELVTTERKTPRVTGGGAMASASTSAGSASSATACTAGVMTRGRLKAESTVAPEEDTDEDSDNEIHNPAVFTWPPWQAGILARNLVPMVATVQGQNLKYQEFFWDANDIYRIFAELEGVWQPAAQPKRRRHRQDALPGPCIASTPKKHRG

SEQ ID NO: 70 - protein, 900 aa, Human cytomegalovirus
MESRGRRCPEMISVLGPISGHVLKAVFSRGDTPVLPHETRLLQTGIHVRVSQPSLILVSQYTPDSTPCHRGDNQLQVQHTYFTGSEVENVSVNVHNPTGRSICPSQEPMSIYVYALPLKMLNIPSINVHHYPSAAERKHRHLPVADAVIHASGKQMWQARLTVSGLAWTRQQNQWKEPDVYYTSAFVFPTKDVALRHVVCAHELVCSMENTRATKMQVIGDQYVKVYLESFCEDVPSGKLFMHVTLGSDVEEDLTMTRNPQPFMRPHERNGFTVLCPKNMIIKPGKISHIMLDVAFTSHEHFGLLCPKSIPGLSISGNLLMNGQQIFLEVQAIRETVELRQYDPVAALFFFDIDLLLQRGPQYSEHPTFTSQYRIQGKLEYRHTWDRHDEGAAQGDDDVWTSGSDSDEELVTTERKTPRVTGGGAMASASTSAGRKRKSASSATACTAGVMTRGRLKAESTVAPEEDTDEDSDNEIHNPAVFTWPPWQAGILARNLVPMVATVQGQNLKYQEFFWDANDIYRIFAELEGVWQPAAQPKRRRHRQDALPGPCIASTPKKHRGESSAKRKMDPDNPDEGPSSKVPRPETPVTKATTFLQTMLRKEVNSQLSLGDPLFPELAEESLKTFEQVTEDCNENPEKDVLTELVKQIKVRVDMVRHRIKEHMLKKYTQTEEKFTGAFNMMGGCLQNALDILDKVHEPFEDMKCIGLTMQSMYENYIVPEDKREMWMACIKELHDVSKGAANKLGGALQAKARAKKDELRRKMMYMCYRNIEFFTKNSAFPKTTNGCSQAMAALQNLPQCSPDEIMSYAQKIFKILDEERDKVLTHIDHIFMDILTTCVETMCNEYKVTSDACMMTMYGGISLLSEFCRVLCCYVLEETSVMLAKRPLITKPEVISVMK

SEQ ID NO: 71 - protein, 1047 aa, synthetic construct
MESRGRRCPEMISVLGPISGHVLKAVFSRGDTPVLPHETRLLQTGIHVRVSQPSLILVSQYTPDSTPCHRGDNQLQVQHTYFTGSEVENVSVNVHNPTGRSICPSQEPMSIYVYALPLKMLNIPSINVHHYPSAAERKHRHLPVADAVIHASGKQMWQARLTVSGLAWTRQQNQWKEPDVYYTSAFVFPTKDVALRHVVCAHELVCSMENTRATKMQVIGDQYVKVYLESFCEDVPSGKLFMHVTLGSDVEEDLTMTRNPQPFMRPHERNGFTVLCPKNMIIKPGKISHIMLDVAFTSHEHFGLLCPKSIPGLSISGNLLMNGQQIFLEVQAIRETVELRQYDPVAALFFFDIDLLLQRGPQYSEHPTFTSQYRIQGKLEYRHTWDRHDEGAAQGDDDVWTSGSDSDEELVTTERKTPRVTGGGAMASASTSAGSASSATACTAGVMTRGRLKAESTVAPEEDTDEDSDNEIHNPAVFTWPPWQAGILARNLVPMVATVQGQNLKYQEFFWDANDIYRIFAELEGVWQPAAQPKRRRHRQDALPGPCIASTPKKHRGESSAKRKMDPDNPDEGPSSKVPRPETPVTKATTFLQTMLRKEVNSQLSLGDPLFPELAEESLKTFEQVTEDCNENPEKDVLTELVKQIKVRVDMVRHRIKEHMLKKYTQTEEKFTGAFNMMGGCLQNALDILDKVHEPFEDMKCIGLTMQSMYENYIVPEDKREMWMACIKELHDVSKGAANKLGGALQAKARAKKDELRRKMMYMCYRNIEFFTKNSAFPKTTNGCSQAMAALQNLPQCSPDEIMSYAQKIFKILDEERDKVLTHIDHIFMDILTTCVETMCNEYKVTSDACMMTMYGGISLLSEFCRVLCCYVLEETSVMLAKRPLITKPEVISVMKRRIEEICMKVFAQYILGADPLRVCSPSVDDLRAIAEESDEEEAIVAYTLATAGASSSDSLVSPPESPVPATIPLSSVIVAENSDQEESEQSDEEQEEGAQEEREDTVSVKSEPVSEIEEVASEEEEDGAEEPTASGGKSTHPMVTRSKADQ

SEQ ID NO: 72 - protein, 491 aa, Human cytomegalovirus
MESSAKRKMDPDNPDEGPSSKVPRPETPVTKATTFLQTMLRKEVNSQLSLGDPLFPELAEESLKTFEQVTEDCNENPEKDVLTELVKQIKVRVDMVRHRIKEHMLKKYTQTEEKFTGAFNMMGGCLQNALDILDKVHEPFEDMKCIGLTMQSMYENYIVPEDKREMWMACIKELHDVSKGAANKLGGALQAKARAKKDELRRKMMYMCYRNIEFFTKNSAFPKTTNGCSQAMAALQNLPQCSPDEIMSYAQKIFKILDEERDKVLTHIDHIFMDILTTCVETMCNEYKVTSDACMMTMYGGISLLSEFCRVLCCYVLEETSVMLAKRPLITKPEVISVMKRRIEEICMKVFAQYILGADPLRVCSPSVDDLRAIAEESDEEEAIVAYTLATAGASSSDSLVSPPESPVPATIPLSSVIVAENSDQEESEQSDEEQEEGAQEEREDTVSVKSEPVSEIEEVASEEEEDGAEEPTASGGKSTHPMVTRSKADQ

SEQ ID NO: 73 - protein, 580 aa, Human cytomegalovirus
MESSAKRKMDPDNPDEGPSSKVPRPETPVTKATTFLQTMLRKEVNSQLSLGDPLFPELAEESLKTFEQVTEDCNENPEKDVLTELGDILAQAVNHAGIDSSSTGPTLTTHSCSVSSAPLNKPTPTSVAVTNTPLPGASATPELSPRKKPRKTTRPFKVIIKPPVPPAPIMLPLIKQEDIKPEPDFTIQYRNKIIDTAGCIVISDSEEEQGEEVETRGATASSPSTGSGTPRVTSPTHPLSQMNHPPLPDPLGRPDEDSSSSSSSSCSSASDSESESEEMKCSSGGGASVTSSHHGRGGFGGAASSSLLSCGHQSSGGASTGPRKKKSKRISELDNEKVRNIMKDKNTPFCTPNVQTRRGRVKIDEVSRMFRHTNRSLEYKNLPFMIPSMHQVLEEAIKVCKTMQVNNKGIQIIYTRNHEVKNEVDQVRCRLGSMCNLALSTPFLMEHTMPVTHPPDVAQRTADACNDGVKAVWNLKELHTHQLCPRSSDYRNMIIHAATPVDLLGALNLCLPLMQKFPKQVMVRIFSTNQGGFMLPIYETAAKAYAVGQFEKPTETPPEDLDTLSLAIEAAIQDLRNKSQ

SEQ ID NO: 74 - protein, 561 aa, Human cytomegalovirus
MESRGRRCPEMISVLGPISGHVLKAVFSRGDTPVLPHETRLLQTGIHVRVSQPSLILVSQYTPDSTPCHRGDNQLQVQHTYFTGSEVENVSVNVHNPTGRSICPSQEPMSIYVYALPLKMLNIPSINVHHYPSAAERKHRHLPVADAVIHASGKQMWQARLTVSGLAWTRQQNQWKEPDVYYTSAFVFPTKDVALRHVVCAHELVCSMENTRATKMQVIGDQYVKVYLESFCEDVPSGKLFMHVTLGSDVEEDLTMTRNPQPFMRPHERNGFTVLCPKNMIIKPGKISHIMLDVAFTSHEHFGLLCPKSIPGLSISGNLLMNGQQIFLEVQAIRETVELRQYDPVAALFFFDIDLLLQRGPQYSEHPTFTSQYRIQGKLEYRHTWDRHDEGAAQGDDDVWTSGSDSDEELVTTERKTPRVTGGGAMASASTSAGRKRKSASSATACTAGVMTRGRLKAESTVAPEEDTDEDSDNEIHNPAVFTWPPWQAGILARNLVPMVATVQGQNLKYQEFFWDANDIYRIFAELEGVWQPAAQPKRRRHRQDALPGPCIASTPKKHRG

SEQ ID NO: 75 - protein, 698 aa, Human cytomegalovirus
MESRIWCLVVCVNLCIVCLGAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDDYSNTHSTRYVTVKDQWHSRGSTWLYRETCNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNRTKRSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLENTDFRVLELYSQKELRSSNVFDLEEIMREFNSYKQHHHHHH

SEQ ID NO: 76 - protein, 8 aa, synthetic construct
CGADPQLC

SEQ ID NO: 77 - protein, 8 aa, synthetic construct
SGCDPQLC

SEQ ID NO: 78 - protein, 5 aa, synthetic construct
RLNPP

SEQ ID NO: 79 - protein, 5 aa, synthetic construct
GADPQ

SEQ ID NO: 80 - protein, 6 aa, unidentified
GMTPEQ

SEQ ID NO: 81 - protein, 10 aa, synthetic construct
YDVCDPQLCY

SEQ ID NO: 82 - protein, 9 aa, synthetic construct
VGPPPTHKI

SEQ ID NO: 83 - protein, 28 aa, synthetic construct
DTYLSAIEDKIEEILSKIYHIENEIARI

SEQ ID NO: 84 - protein, 27 aa, synthetic construct
GYIPEAPRDGQAYVRKDGEWVLLSTFL

SEQ ID NO: 85 - protein, 24 aa, synthetic construct
KIEEILSKQYHIENEIARIKKLIG

SEQ ID NO: 86 - protein, 19 aa, synthetic construct
LKQIVLRIMEIEARIAKIE

SEQ ID NO: 87 - protein, 49 aa, synthetic construct
LKQIVLRIMEIEARIAKIEGSGYIPEAPRDGQAYVRKDGEWVLLSTFLG

SEQ ID NO: 88 - protein, 44 aa, synthetic construct
LKQIVLRIMEIEARIAKIEGSEFNSLKQIVLRIMEIEARIAKIE

SEQ ID NO: 89 - protein, 40 aa, synthetic construct
LKQIVLRIMEIEARIAKIEGSLKQIVLRIMEIEARIAKIE

SEQ ID NO: 90 - protein, 46 aa, synthetic construct
LKQIVLRIMEIEARIAKIEGSLELIKLRIMEIEARIAKIEKDRAIL

SEQ ID NO: 91 - DNA, 10 bp, synthetic construct
  source 1..10: mol_type = other DNA
aacgttcgag

SEQ ID NO: 92 - DNA, 22 bp, synthetic construct
  source 1..22: mol_type = other DNA
tgactgtgaacgttcgagatga

SEQ ID NO: 93 - DNA, 11 bp, synthetic construct
  source 1..11: mol_type = other DNA
  modified_base order(3,7,11): mod_base = OTHER; note = g is 2'-deoxy-7-deazaguanosine
tcgaacgttcg